SEQUENCE LISTING (138 sequences)

SEQ ID NO: 1 - DNA, 20 bp, synthetic construct
  misc_feature 1..20: note = synthetic
  source 1..20: mol_type = other DNA
atccattcatcatggtgtgg

SEQ ID NO: 2 - DNA, 20 bp, synthetic construct
  misc_feature 1..20: note = synthetic
  source 1..20: mol_type = other DNA
tggacaggtggcgctcctct

SEQ ID NO: 3 - DNA, 20 bp, synthetic construct
  misc_feature 1..20: note = synthetic
  source 1..20: mol_type = other DNA
ttggacaggtggcgctcctc

SEQ ID NO: 4 - DNA, 20 bp, synthetic construct
  misc_feature 1..20: note = synthetic
  source 1..20: mol_type = other DNA
gtagccgttctgctgcatct

SEQ ID NO: 5 - DNA, 20 bp, synthetic construct
  misc_feature 1..20: note = synthetic
  source 1..20: mol_type = other DNA
tgctcaaagaacttgtaggt

SEQ ID NO: 6 - DNA, 20 bp, synthetic construct
  misc_feature 1..20: note = synthetic
  source 1..20: mol_type = other DNA
atccatccatcatggcgtgg

SEQ ID NO: 7 - DNA, 20 bp, synthetic construct
  misc_feature 1..20: note = synthetic
  source 1..20: mol_type = other DNA
tggagagatggcgctcctct

SEQ ID NO: 8 - DNA, 20 bp, synthetic construct
  misc_feature 1..20: note = synthetic
  source 1..20: mol_type = other DNA
ttggagagatggcgctcctc

SEQ ID NO: 9 - DNA, 20 bp, synthetic construct
  misc_feature 1..20: note = synthetic
  source 1..20: mol_type = other DNA
atatccgttctgctgcatct

SEQ ID NO: 10 - DNA, 20 bp, synthetic construct
  misc_feature 1..20: note = synthetic
  source 1..20: mol_type = other DNA
tgctcaaagaacttgtaagt

SEQ ID NO: 11 - DNA, 2088 bp, Homo sapiens
  source 1..2088: mol_type = genomic DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 12 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 13 - DNA, 2088 bp, Mus musculus
  source 1..2088: mol_type = genomic DNA
atgctgcccagcttggcactgctcctgctggccgcctggacggttcgggctctggaggtacccactgatggcaacgccgggctgctggcagaaccccagatcgccatgttctgtggtaaactcaacatgcacatgaatgtgcagaatggaaagtgggagtcagacccgtcagggaccaaaacctgcattggcaccaaggagggcatcttgcagtactgccaagaggtctaccctgaactgcagatcacaaacgtggtggaagccaaccagccagtgaccatccagaactggtgcaagcggggccgcaagcagtgcaagacacacacccacatcgtgattccttaccgttgcctagttggtgagtttgtgagcgacgcccttctcgtgcccgacaagtgcaagttcctacaccaggagcggatggatgtttgtgagacccatcttcactggcacaccgtcgccaaagagacatgcagcgagaagagcactaacttgcacgactatggcatgctgctgccctgcggcatcgacaagttccgaggggtagagtttgtatgctgcccgttggccgaggaaagcgacagcgtggattctgcggatgcagaggaggatgactctgatgtctggtggggtggagcggacacagactacgctgatggcggtgaagacaaagtagtagaagtcgccgaagaggaggaagtggctgatgttgaggaagaggaagctgatgatgatgaggatgtggaggatggggacgaggtggaggaggaggccgaggagccctacgaagaggccaccgagagaacaaccagcactgccaccaccaccacaaccaccactgagtccgtggaggaggtggtccgagttcccacgacagcagccagcacccccgacgccgtcgacaagtacctggagacacccggggacgagaacgagcatgcccatttccagaaagccaaagagaggctggaagccaagcaccgagagagaatgtcccaggtcatgagagaatgggaagaggcagagcgtcaagccaagaacttgcccaaagctgacaagaaggccgttatccagcatttccaggagaaagtggaatctctggaacaggaagcagccaatgagagacagcagcttgtagagacacacatggccagagttgaagccatgctcaatgaccgccgccgcctggccctcgagaattacatcactgcactgcaggcggtgcccccaaggcctcatcatgtgttcaacatgctgaagaagtacgtccgtgcggagcagaaagacagacagcacaccctaaagcattttgaacatgtgcgcatggtggaccccaagaaagctgctcagatccggtcccaggttatgacacacctccgtgtgatctacgagcgcatgaaccagtctctgtccctgctctacaatgtccctgcggtggctgaggagattcaagatgaagtcgatgagctgcttcagaaggagcagaactactccgacgatgtcttggccaacatgatcagtgagcccagaatcagctacggaaacgacgctctcatgccttcgctgacggaaaccaagaccaccgtggagctccttcccgtgaatggggaattcagcctggatgacctccagccgtggcacccttttggggtggactctgtgccagccaataccgaaaatgaagtcgagcctgttgacgcccgccccgctgctgaccgaggactgaccactcgaccaggttctgggctgacaaacatcaagacggaagagatctcggaagtgaagatggatgcagaattcggacatgattcaggatttgaagtccgccatcaaaaactggtgttctttgctgaagatgtgggttcgaacaaaggcgccatcatcggactcatggtgggcggcgttgtcatagcaaccgtgattgtcatcaccctggtgatgttgaagaagaaacagtacacatccatccatcatggcgtggtggaggtcgacgccgccgtgaccccagaggagcgccatctctccaagatgcagcagaacggatatgagaatccaacttacaagttctttgagcaaatgcagaactaa

SEQ ID NO: 14 - protein, 695 aa, Mus musculus
MLPSLALLLLAAWTVRALEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 15 - DNA, 4176 bp, Streptococcus pyogenes
  source 1..4176: mol_type = genomic DNA
atgggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaag

SEQ ID NO: 16 - DNA, 80 bp, synthetic construct
  misc_feature 1..80: note = synthetic
  source 1..80: mol_type = other DNA
gttttagagctagaaatagcaagttaaaataaggctagtccgttatcaacttgaaaaagtggcaccgagtcggtgctttt

SEQ ID NO: 17 - DNA, 9286 bp, synthetic construct
  misc_feature 1..9286: note = synthetic
  source 1..9286: mol_type = other DNA
ccccacgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatccattcatcatggtgtgggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctcagacggccccgtaatgcagaagaaaaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 18 - DNA, 7400 bp, synthetic construct
  misc_feature 1..7400: note = synthetic
  source 1..7400: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagaaagcttagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcaccggtgccaccatgtacccatacgatgttccagattacgcttcgccgaagaaaaagcgcaaggtcgaagcgtccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacagccccaagaagaagagaaaggtggaggccagctaagaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 19 - DNA, 5305 bp, synthetic construct
  misc_feature 1..5305: note = synthetic
  source 1..5305: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccatccatccatcatggcgtgggttttagagctagaaatagcaagttaaaataaggctagtccgttatcaacttgaaaaagtggcaccgagtcggtgctttttttctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccggatcctctagagtcgacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagaggaggaggaggagacagacagcaggatgccccacctcgacagccccggcagctcccagccgagacgctccttcctctcaagggtgatcagggcagcgctaccgttgcagctgcttctgctgctgctgctgctcctggcctgcctgctacctgcctctgaagatgactacagctgcacccaggccaacaactttgcccgatccttctaccccatgctgcggtacaccaacgggccacctcccacctaggaattcgatatcaagcttatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 20 - DNA, 13013 bp, synthetic construct
  misc_feature 1..13013: note = synthetic
  source 1..13013: mol_type = other DNA
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatccattcatcatggtgtgggttttagagctagaaatagcaagttaaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgg

SEQ ID NO: 21 - DNA, 22 bp, synthetic construct
  misc_feature 1..22: note = synthetic
  source 1..22: mol_type = other DNA
aggaacggagtgacctgtttcc

SEQ ID NO: 22 - DNA, 22 bp, synthetic construct
  misc_feature 1..22: note = synthetic
  source 1..22: mol_type = other DNA
ttcctccatggtaaccacgcat

SEQ ID NO: 23 - DNA, 22 bp, synthetic construct
  misc_feature 1..22: note = synthetic
  source 1..22: mol_type = other DNA
tggggaagccacatgttgtaca

SEQ ID NO: 24 - DNA, 22 bp, synthetic construct
  misc_feature 1..22: note = synthetic
  source 1..22: mol_type = other DNA
atgttttggtgggccatttggt

SEQ ID NO: 25 - DNA, 26 bp, synthetic construct
  misc_feature 1..26: note = synthetic
  source 1..26: mol_type = other DNA
aaattatgggtgttctgcaatcttgg

SEQ ID NO: 26 - DNA, 23 bp, synthetic construct
  misc_feature 1..23: note = synthetic
  source 1..23: mol_type = other DNA
acttgtgttacagcacagctgtc

SEQ ID NO: 27 - DNA, 21 bp, synthetic construct
  misc_feature 1..21: note = synthetic
  source 1..21: mol_type = other DNA
gccctccagaagtattggctt

SEQ ID NO: 28 - DNA, 21 bp, synthetic construct
  misc_feature 1..21: note = synthetic
  source 1..21: mol_type = other DNA
gtcagggccttgctctacaaa

SEQ ID NO: 29 - DNA, 20 bp, synthetic construct
  misc_feature 1..20: note = synthetic
  source 1..20: mol_type = other DNA
cgcaaaaactggctgcgtat

SEQ ID NO: 30 - DNA, 20 bp, synthetic construct
  misc_feature 1..20: note = synthetic
  source 1..20: mol_type = other DNA
tgtaggcgcacatgcagaag

SEQ ID NO: 31 - DNA, 21 bp, synthetic construct
  misc_feature 1..21: note = synthetic
  source 1..21: mol_type = other DNA
caggtagagcgtggaaactca

SEQ ID NO: 32 - DNA, 20 bp, synthetic construct
  misc_feature 1..20: note = synthetic
  source 1..20: mol_type = other DNA
tgtgcgcattaggaccagat

SEQ ID NO: 33 - DNA, 20 bp, synthetic construct
  misc_feature 1..20: note = synthetic
  source 1..20: mol_type = other DNA
cacctgacaatgctgtccca

SEQ ID NO: 34 - DNA, 21 bp, synthetic construct
  misc_feature 1..21: note = synthetic
  source 1..21: mol_type = other DNA
agacaaggtctgtctccttgc

SEQ ID NO: 35 - DNA, 20 bp, synthetic construct
  misc_feature 1..20: note = synthetic
  source 1..20: mol_type = other DNA
ccaactctttgcttaggggc

SEQ ID NO: 36 - DNA, 20 bp, synthetic construct
  misc_feature 1..20: note = synthetic
  source 1..20: mol_type = other DNA
atcgtccctggtgcattctc

SEQ ID NO: 37 - DNA, 20 bp, synthetic construct
  misc_feature 1..20: note = synthetic
  source 1..20: mol_type = other DNA
ggaaaaccaggtagaggggg

SEQ ID NO: 38 - DNA, 20 bp, synthetic construct
  misc_feature 1..20: note = synthetic
  source 1..20: mol_type = other DNA
tctctggctcgagggtacat

SEQ ID NO: 39 - DNA, 20 bp, synthetic construct
  misc_feature 1..20: note = synthetic
  source 1..20: mol_type = other DNA
ctgcatgccatgggtaggta

SEQ ID NO: 40 - DNA, 19 bp, synthetic construct
  misc_feature 1..19: note = synthetic
  source 1..19: mol_type = other DNA
caggctgtttcgggtcctt

SEQ ID NO: 41 - DNA, 21 bp, synthetic construct
  misc_feature 1..21: note = synthetic
  source 1..21: mol_type = other DNA
agactcttctccgattccagc

SEQ ID NO: 42 - DNA, 20 bp, synthetic construct
  misc_feature 1..20: note = synthetic
  source 1..20: mol_type = other DNA
tccagcacgatctggtaggc

SEQ ID NO: 43 - DNA, 22 bp, synthetic construct
  misc_feature 1..22: note = synthetic
  source 1..22: mol_type = other DNA
agtgcttttctttgcctttgct

SEQ ID NO: 44 - DNA, 20 bp, synthetic construct
  misc_feature 1..20: note = synthetic
  source 1..20: mol_type = other DNA
tgctcgggaggtgtttctac

SEQ ID NO: 45 - DNA, 20 bp, synthetic construct
  misc_feature 1..20: note = synthetic
  source 1..20: mol_type = other DNA
aacaaggcagctcctcaact

SEQ ID NO: 46 - DNA, 21 bp, synthetic construct
  misc_feature 1..21: note = synthetic
  source 1..21: mol_type = other DNA
gacgtcagaattgagggtgga

SEQ ID NO: 47 - DNA, 22 bp, synthetic construct
  misc_feature 1..22: note = synthetic
  source 1..22: mol_type = other DNA
ccagcgggatgaactggtaaga

SEQ ID NO: 48 - DNA, 22 bp, synthetic construct
  misc_feature 1..22: note = synthetic
  source 1..22: mol_type = other DNA
cccaggtcaccttaaggagcaa

SEQ ID NO: 49 - DNA, 22 bp, synthetic construct
  misc_feature 1..22: note = synthetic
  source 1..22: mol_type = other DNA
gagagagttggaggccttgagg

SEQ ID NO: 50 - DNA, 22 bp, synthetic construct
  misc_feature 1..22: note = synthetic
  source 1..22: mol_type = other DNA
aaccacagtgacaagtggctct

SEQ ID NO: 51 - DNA, 22 bp, synthetic construct
  misc_feature 1..22: note = synthetic
  source 1..22: mol_type = other DNA
gtgaatgcgtctgttccaaggg

SEQ ID NO: 52 - DNA, 22 bp, synthetic construct
  misc_feature 1..22: note = synthetic
  source 1..22: mol_type = other DNA
gctgctgggactatctgggaat

SEQ ID NO: 53 - DNA, 22 bp, synthetic construct
  misc_feature 1..22: note = synthetic
  source 1..22: mol_type = other DNA
ttttaggggctcgaccttccag

SEQ ID NO: 54 - DNA, 22 bp, synthetic construct
  misc_feature 1..22: note = synthetic
  source 1..22: mol_type = other DNA
tgcactaatttcccagggctca

SEQ ID NO: 55 - DNA, 30 bp, synthetic construct
  misc_feature 1..30: note = synthetic
  source 1..30: mol_type = other DNA
cacatccatccatcatggcgtggtggaggt

SEQ ID NO: 56 - DNA, 30 bp, synthetic construct
  misc_feature 1..30: note = synthetic
  source 1..30: mol_type = other DNA
tcttggagagatggcgctcctctggggtca

SEQ ID NO: 57 - DNA, 30 bp, synthetic construct
  misc_feature 1..30: note = synthetic
  source 1..30: mol_type = other DNA
ctcatatccgttctgctgcatcttggagag

SEQ ID NO: 58 - DNA, 303 bp, Mus musculus
  source 1..303: mol_type = genomic DNA
atggatgcagaattcggacatgattcaggatttgaagtccgccatcaaaaactggtgttctttgctgaagatgtgggttcgaacaaaggcgccatcatcggactcatggtgggcggcgttgtcatagcaaccgtgattgtcatcaccctggtgatgttgaagaagaaacagtacacatccatccatcatggcgtggtggaggtcgacgccgccgtgaccccagaggagcgccatctctccaagatgcagcagaacggatatgagaatccaacttacaagttctttgagcaaatgcagaactaa

SEQ ID NO: 59 - protein, 100 aa, Mus musculus
MDAEFGHDSGFEVRHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 60 - DNA, 66 bp, synthetic construct
  misc_feature 1..66: note = synthetic
  source 1..66: mol_type = other DNA
gaagaagaaacagtacacatccatccatcatggcgtggtggaggtaggtaaacctggaggcttgtc

SEQ ID NO: 61 - DNA, 46 bp, synthetic construct
  misc_feature 1..46: note = synthetic
  source 1..46: mol_type = other DNA
aacagtacacatccatccatcatggcgtggtggaggtaggtaaacc

SEQ ID NO: 62 - DNA, 41 bp, synthetic construct
  misc_feature 1..41: note = synthetic
  source 1..41: mol_type = other DNA
aacagtacacatccatccatcatggtggaggtaggtaaacc

SEQ ID NO: 63 - DNA, 41 bp, synthetic construct
  misc_feature 1..41: note = synthetic
  source 1..41: mol_type = other DNA
aacagtacacatccatccatcatggcggaggtaggtaaacc

SEQ ID NO: 64 - DNA, 38 bp, synthetic construct
  misc_feature 1..38: note = synthetic
  source 1..38: mol_type = other DNA
aacagtacacatccatccatcatggaggtaggtaaacc

SEQ ID NO: 65 - DNA, 44 bp, synthetic construct
  misc_feature 1..44: note = synthetic
  source 1..44: mol_type = other DNA
aacagtacacatccatccatcatggcggtggaggtaggtaaacc

SEQ ID NO: 66 - DNA, 44 bp, synthetic construct
  misc_feature 1..44: note = synthetic
  source 1..44: mol_type = other DNA
aacagtacacatccatccatcatggtggtggaggtaggtaaacc

SEQ ID NO: 67 - DNA, 23 bp, Mus musculus
  source 1..23: mol_type = genomic DNA
atccatccatcatggcgtggtgg

SEQ ID NO: 68 - DNA, 23 bp, Homo sapiens
  source 1..23: mol_type = genomic DNA
atccattcatcatggtgtggtgg

SEQ ID NO: 69 - DNA, 66 bp, synthetic construct
  misc_feature 1..66: note = synthetic
  source 1..66: mol_type = other DNA
gaagaagaaacagtacacatccattcatcatggtgtggtggaggtaggtaaacttgactgcatgtt

SEQ ID NO: 70 - DNA, 45 bp, synthetic construct
  misc_feature 1..45: note = synthetic
  source 1..45: mol_type = other DNA
aacagtacacatccattcatcatggtgtggtggaggtaggtaaac

SEQ ID NO: 71 - DNA, 40 bp, synthetic construct
  misc_feature 1..40: note = synthetic
  source 1..40: mol_type = other DNA
aacagtacacatccattcatcatggtggaggtaggtaaac

SEQ ID NO: 72 - DNA, 43 bp, synthetic construct
  misc_feature 1..43: note = synthetic
  source 1..43: mol_type = other DNA
aacagtacacatccattcatcatggtggtggaggtaggtaaac

SEQ ID NO: 73 - DNA, 41 bp, synthetic construct
  misc_feature 1..41: note = synthetic
  source 1..41: mol_type = other DNA
aacagtacacatccattcatcattggtggaggtaggtaaac

SEQ ID NO: 74 - DNA, 44 bp, synthetic construct
  misc_feature 1..44: note = synthetic
  source 1..44: mol_type = other DNA
aacagtacacatccattcatcatggtgggtggaggtaggtaaac

SEQ ID NO: 75 - DNA, 39 bp, synthetic construct
  misc_feature 1..39: note = synthetic
  source 1..39: mol_type = other DNA
aacagtacacatccattcatctggtggaggtaggtaaac

SEQ ID NO: 76 - protein, 46 aa, synthetic construct
  REGION 1..46: note = synthetic
GGVVIATVIVITLVMLKKKQYTSIHHGVVEVDAAVTPEERHLSKMQ

SEQ ID NO: 77 - protein, 29 aa, synthetic construct
  REGION 1..29: note = synthetic
GGVVIATVIVITLVMLKKKQYTSIHHGGG

SEQ ID NO: 78 - protein, 30 aa, synthetic construct
  REGION 1..30: note = synthetic
GGVVIATVIVITLVMLKKKQYTSIHHGGGG

SEQ ID NO: 79 - protein, 26 aa, synthetic construct
  REGION 1..26: note = synthetic
GGVVIATVIVITLVMLKKKQYTSIHH

SEQ ID NO: 80 - protein, 27 aa, synthetic construct
  REGION 1..27: note = synthetic
GGVVIATVIVITLVMLKKKQYTSIHHG

SEQ ID NO: 81 - protein, 44 aa, synthetic construct
  REGION 1..44: note = synthetic
GGVVIATVIVITLVMLKKKQYTSIHLVEVDAAVTPEERHLSKMQ

SEQ ID NO: 82 - protein, 37 aa, synthetic construct
  REGION 1..37: note = synthetic
GVVEVDAAVTPEERHLSKMQQNGYENPTYKFFEQMQN

SEQ ID NO: 83 - DNA, 45 bp, synthetic construct; identical to SEQ ID NO: 70
  misc_feature 1..45: note = synthetic
  source 1..45: mol_type = other DNA

SEQ ID NO: 84 - DNA, 40 bp, synthetic construct; identical to SEQ ID NO: 71
  misc_feature 1..40: note = synthetic
  source 1..40: mol_type = other DNA

SEQ ID NO: 85 - DNA, 43 bp, synthetic construct; identical to SEQ ID NO: 72
  misc_feature 1..43: note = synthetic
  source 1..43: mol_type = other DNA

SEQ ID NO: 86 - DNA, 44 bp, synthetic construct; identical to SEQ ID NO: 74
  misc_feature 1..44: note = synthetic
  source 1..44: mol_type = other DNA

SEQ ID NO: 87 - DNA, 41 bp, synthetic construct; identical to SEQ ID NO: 73
  misc_feature 1..41: note = synthetic
  source 1..41: mol_type = other DNA

SEQ ID NO: 88 - DNA, 42 bp, synthetic construct
  misc_feature 1..42: note = synthetic
  source 1..42: mol_type = other DNA
aacagtacacatccattcatcatgtggtggaggtaggtaaac

SEQ ID NO: 89 - DNA, 45 bp, synthetic construct
  misc_feature 1..45: note = synthetic
  source 1..45: mol_type = other DNA
tgctgcatcttggacaggtggcgctcctctggggtgacagcggcg

SEQ ID NO: 90 - DNA, 46 bp, synthetic construct
  misc_feature 1..46: note = synthetic
  variation 28: note = n is a, c, g, or t
  source 1..46: mol_type = other DNA
tgctgcatcttggacaggtggcgctccntctggggtgacagcggcg

SEQ ID NO: 91 - DNA, 43 bp, synthetic construct
  misc_feature 1..43: note = synthetic
  source 1..43: mol_type = other DNA
tgctgcatcttggacaggtggcgcttctggggtgacagcggcg

SEQ ID NO: 92 - DNA, 40 bp, synthetic construct
  misc_feature 1..40: note = synthetic
  source 1..40: mol_type = other DNA
tgctgcatcttggacaggtggctctggggtgacagcggcg

SEQ ID NO: 93 - DNA, 42 bp, synthetic construct
  misc_feature 1..42: note = synthetic
  source 1..42: mol_type = other DNA
tgctgcatcttggacaggtggcgctctggggtgacagcggcg

SEQ ID NO: 94 - DNA, 44 bp, synthetic construct
  misc_feature 1..44: note = synthetic
  source 1..44: mol_type = other DNA
tgctgcatcttggacaggtggcgctctctggggtgacagcggcg

SEQ ID NO: 95 - DNA, 45 bp, synthetic construct
  misc_feature 1..45: note = synthetic
  source 1..45: mol_type = other DNA
ttggattttcgtagccgttctgctgcatcttggacaggtggcgct

SEQ ID NO: 96 - DNA, 46 bp, synthetic construct
  misc_feature 1..46: note = synthetic
  variation 28: note = n is a, c, g, or t
  source 1..46: mol_type = other DNA
ttggattttcgtagccgttctgctgcantcttggacaggtggcgct

SEQ ID NO: 97 - DNA, 36 bp, synthetic construct
  misc_feature 1..36: note = synthetic
  source 1..36: mol_type = other DNA
ttggattttcgtagccgttcttggacaggtggcgct

SEQ ID NO: 98 - DNA, 44 bp, synthetic construct
  misc_feature 1..44: note = synthetic
  source 1..44: mol_type = other DNA
ttggattttcgtagccgttctgctgctcttggacaggtggcgct

SEQ ID NO: 99 - DNA, 42 bp, synthetic construct
  misc_feature 1..42: note = synthetic
  source 1..42: mol_type = other DNA
ttggattttcgtagccgttctgcttcttggacaggtggcgct

SEQ ID NO: 100 - DNA, 39 bp, synthetic construct
  misc_feature 1..39: note = synthetic
  source 1..39: mol_type = other DNA
ttggattttcgtagccgttcttcttggacaggtggcgct

SEQ ID NO: 101 - protein, 46 aa, synthetic construct; identical to SEQ ID NO: 76
  REGION 1..46: note = synthetic

SEQ ID NO: 102 - protein, 29 aa, synthetic construct
  REGION 1..29: note = synthetic
GGVVIATVIVITLVMLKKKQYTSIHHGGG

SEQ ID NO: 103 - protein, 30 aa, synthetic construct; identical to SEQ ID NO: 78
  REGION 1..30: note = synthetic

SEQ ID NO: 104 - protein, 27 aa, synthetic construct
  REGION 1..27: note = synthetic
GGVVIATVIVITLVMLKKKQYTSIHHG

SEQ ID NO: 105 - protein, 26 aa, synthetic construct
  REGION 1..26: note = synthetic
GGVVIATVIVITLVMLKKKQYTSIHH

SEQ ID NO: 106 - protein, 45 aa, synthetic construct
  REGION 1..45: note = synthetic
GGVVIATVIVITLVMLKKKQYTSIHHVVAVDAAVTPEERHLSKMQ

SEQ ID NO: 107 - protein, 46 aa, synthetic construct
  REGION 1..46: note = synthetic
KKQYTSIHHGVVEVDAAVTPEERHLSKMQQNGYENPTYKFFEQMQN

SEQ ID NO: 108 - protein, 41 aa, synthetic construct
  REGION 1..41: note = synthetic
  VARIANT 21: note = X is D or E
KKQYTSIHHGVVEVDAAVTPXGAPPVQDAAERLRKSNLQVL

SEQ ID NO: 109 - protein, 41 aa, synthetic construct
  REGION 1..41: note = synthetic
KKQYTSIHHGVVEVDAAVTPEARPPVQDAAERLRKSNLQVL

SEQ ID NO: 110 - protein, 39 aa, synthetic construct
  REGION 1..39: note = synthetic
KKQYTSIHHGVVEVDAAVTPEPPVQDAAERLRKSNLQVL

SEQ ID NO: 111 - protein, 45 aa, synthetic construct
  REGION 1..45: note = synthetic
KKQYTSIHHGVVEVDAAVTPERHLSKMQQNSYENPTYKFFEQMQN

SEQ ID NO: 112 - protein, 20 aa, synthetic construct
  REGION 1..20: note = synthetic
KKQYTSIHHGVVEVDAAVTP

SEQ ID NO: 113 - protein, 27 aa, synthetic construct
  REGION 1..27: note = synthetic
KKQYTSIHHGVVEVDAAVTPEERHLSK

SEQ ID NO: 114 - protein, 43 aa, synthetic construct
  REGION 1..43: note = synthetic
KKQYTSIHHGVVEVDAAVTPEERHLSKNGYENPTYKFFEQMQN

SEQ ID NO: 115 - protein, 27 aa, synthetic construct
  REGION 1..27: note = synthetic
KKQYTSIHHGVVEVDAAVTPEERHLSK

SEQ ID NO: 116 - protein, 45 aa, synthetic construct
  REGION 1..45: note = synthetic
KKQYTSIHHGVVEVDAAVTPEERHLSKKQNGYENPTYKFFEQMQN

SEQ ID NO: 117 - protein, 44 aa, synthetic construct
  REGION 1..44: note = synthetic
KKQYTSIHHGVVEVDAAVTPEERHLSKKNGYENPTYKFFEQMQN

SEQ ID NO: 118 - protein, 46 aa, synthetic construct; identical to SEQ ID NO: 76
  REGION 1..46: note = synthetic

SEQ ID NO: 119 - protein, 48 aa, synthetic construct
  REGION 1..48: note = synthetic
GGVVIATVIVITLVMLKKKQYTSIHHGGGRRRRDPRGAPSLQDAAERI

SEQ ID NO: 120 - protein, 47 aa, synthetic construct
  REGION 1..47: note = synthetic
GGVVIATVIVITLVMLKKKQYTSIHHGGRRRRDPRGAPSLQDAAERI

SEQ ID NO: 121 - protein, 49 aa, synthetic construct
  REGION 1..49: note = synthetic
GGVVIATVIVITLVMLKKKQYTSIHHGGGGRRRRDPRGAPSLQDAAERI

SEQ ID NO: 122 - DNA, 20 bp, synthetic construct
  misc_feature 1..20: note = synthetic
  source 1..20: mol_type = other DNA
gtccatccatcatggcctgg

SEQ ID NO: 123 - DNA, 20 bp, synthetic construct
  misc_feature 1..20: note = synthetic
  source 1..20: mol_type = other DNA
ttccatccatcatggcttgg

SEQ ID NO: 124 - DNA, 20 bp, synthetic construct
  misc_feature 1..20: note = synthetic
  source 1..20: mol_type = other DNA
gtccctccatcatggcctgg

SEQ ID NO: 125 - DNA, 20 bp, synthetic construct
  misc_feature 1..20: note = synthetic
  source 1..20: mol_type = other DNA
ggccatcattcatggcgtgg

SEQ ID NO: 126 - DNA, 20 bp, synthetic construct
  misc_feature 1..20: note = synthetic
  source 1..20: mol_type = other DNA
ataaatatatcatggcgtgg

SEQ ID NO: 127 - DNA, 20 bp, synthetic construct
  misc_feature 1..20: note = synthetic
  source 1..20: mol_type = other DNA
ctcccttcatcttggtgtgg

SEQ ID NO: 128 - DNA, 20 bp, synthetic construct
  misc_feature 1..20: note = synthetic
  source 1..20: mol_type = other DNA
atgcaatcagcatggtgtgg

SEQ ID NO: 129 - DNA, 20 bp, synthetic construct
  misc_feature 1..20: note = synthetic
  source 1..20: mol_type = other DNA
ttctgttcagcatggtgtgg

SEQ ID NO: 130 - DNA, 20 bp, synthetic construct
  misc_feature 1..20: note = synthetic
  source 1..20: mol_type = other DNA
ttaaattcaacatggtgtgg

SEQ ID NO: 131 - DNA, 20 bp, synthetic construct
  misc_feature 1..20: note = synthetic
  source 1..20: mol_type = other DNA
agccatttatcaaggtgtgg

SEQ ID NO: 132 - DNA, 23 bp, synthetic construct
  misc_feature 1..23: note = synthetic
  source 1..23: mol_type = other DNA
atccatccatcatggcgtggtgg

SEQ ID NO: 133 - DNA, 23 bp, synthetic construct
  misc_feature 1..23: note = synthetic
  source 1..23: mol_type = other DNA
gactatcagccatggagtggtgg

SEQ ID NO: 134 - DNA, 23 bp, synthetic construct
  misc_feature 1..23: note = synthetic
  source 1..23: mol_type = other DNA
atccatccatcatggcgtggtgg

SEQ ID NO: 135 - DNA, 23 bp, synthetic construct
  misc_feature 1..23: note = synthetic
  source 1..23: mol_type = other DNA
caccatcagccacgggattgtgg

SEQ ID NO: 136 - DNA, 23 bp, synthetic construct
  misc_feature 1..23: note = synthetic
  source 1..23: mol_type = other DNA
atccattcatcatggtgtggtgg

SEQ ID NO: 137 - DNA, 23 bp, synthetic construct
  misc_feature 1..23: note = synthetic
  source 1..23: mol_type = other DNA
ggctatcagccatggcgtggtgg

SEQ ID NO: 138 - DNA, 23 bp, synthetic construct
  misc_feature 1..23: note = synthetic
  source 1..23: mol_type = other DNA
atccattcatcatggtgtggtgg

(19) United States
(12) Patent Application Publication
(10) Pub. No.: US 2026/0097136 A1
(43) Pub. Date: Apr. 9, 2026

(54) GENE EDITING-BASED METHOD OF ATTENUATING THE BETA-AMYLOID PATHWAY

(71) Applicant: Wisconsin Alumni Research Foundation, Madison, WI (US)

(72) Inventors: Subhojit Roy, Madison, WI (US); Jichao Sun, Madison, WI (US)

(73) Assignee: Wisconsin Alumni Research Foundation, Madison, WI (US)

(21) Appl. No.: 19/387,161

(22) Filed: Nov. 12, 2025

Related U.S. Application Data
(63) Continuation of application No. 18/319,042, filed on May 17, 2023, now Pat. No. 12,496,360.
Application No. 18/319,042 is a continuation of application No. 17/494,457, filed on Oct. 5, 2021, now Pat. No. 11,701,436.
Application No. 17/494,457 is a division of application No. 16/251,970, filed on Jan. 18, 2019, now Pat. No. 11,173,216.
(60) Provisional application No. 62/618,694, filed on Jan. 18, 2018.

(51) Int. Cl. A61K 48/00; A61K 9/00; A61P 25/28; C07K 14/47; C12N 9/22; C12N 15/10; C12N 15/90

(52) U.S. Cl. CPC A61K 48/0066; A61K 9/0019; A61K 48/0008; A61K 48/0058; A61K 48/0075; A61P 25/28; C07K 14/4711; C12N 9/22; C12N 15/102; C12N 15/907; C12N 2310/20; C12N 2740/16043; C12N 2750/14143

(57) ABSTRACT

Described herein are CRISPR/Cas9 constructs designed for the C-terminal truncation of human amyloid precursor protein (APP) as well as methods of making and using such a construct.

### CROSS-REFERENCE TO RELATED APPLICATIONS

This application is a continuation of U.S. application Ser. No. 18/319,042, filed on May 17, 2023, which is a continuation of U.S. application Ser. No. 17/494,457, filed on Oct. 5, 2021, now U.S. Pat. No. 11,701,436, which is a divisional of U.S. application Ser. No. 16/251,970, filed on Jan. 18, 2019, now U.S. Pat. No. 11,173,216, which claims priority to U.S. Provisional Patent Application No. 62/618,694, filed Jan. 18, 2018, which are incorporated herein by reference in their entirety.

### STATEMENT REGARDING FEDERALLY SPONSORED RESEARCH

This invention was made with government support under AG048218 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on Dec. 12, 2025, is named “107668_349_sequence_list replacement” and is 218,134 bytes in size. The Sequence Listing does not go beyond the disclosure in the application as filed.

### BACKGROUND

The gradual accumulation of Aβ in brains is a neuropathologic hallmark of Alzheimer's disease (AD). Aβ is generated by the sequential cleavage of the amyloid precursor protein (APP) by β- and γ-secretases (β-secretase aka BACE-1, and γ-secretase), with BACE-1-cleavage as the rate-limiting step. Substantial evidence indicates that accrual of APP-cleavage products plays a key role in AD, making the “amyloidogenic pathway” an important therapeutic target (1-3).

CRISPR/Cas9 gene editing is emerging as a promising tool to disrupt the expression of disease-causing genes or edit pathogenic mutations (4). Originally discovered in bacteria as part of a natural self-defense mechanism, the Cas9 nuclease—guided by a short guide RNA (sgRNA)—generates double-stranded breaks (DSB) at targeted genomic loci (5).

However, to date, the application of gene editing to neurologic diseases has been limited (6). For instance, CRISPR/Cas9 has been used in cell-based models to edit triplet-repeat expansions of Huntington's and Fragile X syndrome (7, 8). Besides significant technical caveats such as low editing efficiency and limited in vivo validation (6), such canonical approaches would only be applicable to the small fraction of cases that are inherited (i.e. <10% of AD, Parkinson's, ALS); with a different approach required for each gene. Moreover, the feasibility of CRISPR/Cas9 as a therapeutic possibility in AD has not been reported.

Needed in the art of Alzheimer's disease treatment is an improved method of using gene editing methods to treat or prevent the disease.

### SUMMARY OF THE INVENTION

In a first aspect, provided herein is a method of treating or preventing Alzheimer's disease (AD) caused by formation of amyloid plaques composed of amyloid beta (Aβ) peptides, wherein the method comprises the steps of (a) obtaining a gene-editing construct specific for the amyloid precursor protein (APP), wherein the construct facilitates truncation of the APP C-terminus when combined with a Cas9 nuclease, and (b) delivering the construct and a construct encoding the Cas9 nuclease to a patient in need of AD therapy, wherein the APP molecule is truncated and production of Aβ peptides is decreased in the patient's brain. In some embodiments, the truncation of the APP C-terminus occurs at an APP residue selected from the group consisting of 659, 670, 676, and 686. In some embodiments, the gene-editing construct comprises a gRNA sequence selected from the group consisting of SEQ ID NOs:1-10. In some embodiments, the construct and the nuclease are delivered in a composition comprising an adeno-associated viral vector and a nanocarrier delivery vehicle. In some embodiments, the composition is delivered intravenously or intrathecally.

In a second aspect, provided herein is a method of reducing the formation of amyloid plaques in a patient's brain, wherein the plaques are comprise amyloid beta (Aβ) peptides, the method comprises the steps of (a) obtaining a gene-editing construct specific for the amyloid precursor protein (APP), wherein the construct facilitates truncation of the APP C-terminus when combined with a Cas9 nuclease, and (b) delivering the construct and nuclease to a patient in need of AD therapy, wherein the APP molecule is truncated and production of Aβ peptides is decreased in the patient's brain. In some embodiments, the truncation of the APP C-terminus occurs at an APP residue selected from the group consisting of 659, 670, 676, and 686. In some embodiments, the gene-editing construct comprises a gRNA sequence selected from the group consisting of SEQ ID NO:1-10. In some embodiments, the construct and the nuclease are delivered in a composition comprising an adeno-associated viral vector and a nanocarrier delivery vehicle. In some embodiments, the composition is delivered intravenously or intrathecally.

In a third aspect, provided herein is a genetic construct comprising, a sequence encoding for a Cas9 nuclease and a sequence encoding a gRNA specific to amyloid precursor protein (APP). In some embodiments, the construct is packaged in a viral vector selected from the group consisting of a lentiviral vector and an adeno-associated viral (AAV) vector. In some embodiments, the construct further comprises at least one neuron specific promoter. In some embodiments, the neuron specific promoter is selected from the group consisting of human synapsin 1 (hSyn1) promoter, and mouse Mecp2 promoter (pMecp2). In some embodiments, the construct further comprises an RNA Pol III promoter. In some embodiments, the RNA Pol III promoter is a U6 promoter. In some embodiments, the sequence of the gRNA is selected from the group consisting of SEQ ID NOs:1-10. In some embodiments, the sequence of the Cas9 nuclease consists of SEQ ID NO: 15. In some embodiments, the construct comprises the sequence of SEQ ID NO:17, SEQ ID NO:18, SEQ ID NO:19, or SEQ ID NO:20. In some embodiments, the sequence encoding for a Cas9 nuclease in packaged on a first AAV vector and the sequence encoding a gRNA specific to amyloid precursor protein (APP) is packaged on a second AAV vector.

In a fourth aspect, provided herein is a kit for reducing the formation of amyloid plaques in a patient's brain, the kit comprising a first viral vector encoding a gRNA selected from the group consisting of SEQ ID NOs:1-10 and a second viral vector encoding a Cas9 nuclease. In some embodiments, the viral vector is selected from the group consisting of a lentiviral vector and an adeno-associated viral (AAV) vector. In some embodiments, the first or second viral vector further comprises at least one neuron specific promoter. In some embodiments, the neuron specific promoter is selected from the group consisting of human synapsin 1 (hSyn1) promoter, and mouse Mecp2 promoter (pMecp2). In some embodiments, the first or second viral vector further comprises an RNA Pol III promoter. In some embodiments, the RNA Pol III promoter is a U6 promoter. In some embodiments, the kit comprises a viral vector encoding both a gRNA selected from the group consisting of SEQ ID NOs:1-10 and a Cas9 nuclease.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1F show manipulation of the amyloid pathway by CRISPR/Cas9 editing. (FIG. 1A) Schematic and C-terminal sequence of mouse APP showing PAM sites (yellow) and genomic targets for the three APP-sgRNAs (APP-659 sgRNA used henceforth and referred to as ‘APP-sgRNA’—see text). Note that the C-terminal antibody Y188 recognizes the last 20 amino acids of APP. (FIG. 1B) Neuro2A cells were transfected with APP-sgRNA and Cas9 (or Cas9 only), and immunostained with the Y188 antibody (after 5 days; mCherry labels transfected cells). Note decreased APP (Y188) fluorescence, indicating APP editing (quantified on right, mean±SEM of 39 cells from two independent experiments per condition, p<0.0001). (FIGS. 1C-1D) Neuro2A cells were transduced by lentiviral vectors carrying APP-sgRNA and Cas9 (or non-targeting control-sgRNA/Cas9 as control) and immunoblotted with Y188 and 22C11 antibodies (latter recognizes APP N-terminus). A gamma secretase inhibitor (GSI) was added to allow detection of accumulated APP CTF's (see methods, GAPDH used as loading controls). Note attenuated signal with the Y188 antibody in APP-sgRNA treated samples, but no change in 22C11 signal. Blots quantified in (d), mean±SEM of six independent experiments, p<0.0001. (FIG. 1E) Time course of APP-editing in neuro2a cells. Cells were transfected with a vector carrying APP-sgRNA and Cas9, and APP-CTFs were analyzed by Western blotting (in the presence of GSI). (FIG. 1F) Deep sequencing of APP C-terminus in neuro2A cells. Top: Frequency of base-pair matches between gRNA-edited and WT mouse sequence. Red underline marks the sgRNA target sequence and arrowhead denotes predicted cut-site. Note extensive mismatch around predicted cut-site, indicating robust editing. Bottom: Major mutated APP loci resulting from sgRNA-editing, and their frequencies.

FIGS. 2A-2H show gene editing of APP C-terminus and effects on APP processing in human cells. (FIG. 2A) Comparison of mouse and human APP-sgRNA targeting sequences (red arrowheads indicate differences; yellow bar denotes the PAM site). (FIG. 2B) Human iPSC-derived NPCs were transduced by lentiviral vectors carrying APP-sgRNA and Cas9 (or non-targeting control-sgRNA/Cas9 as control) and differentiated into neurons. After 3 weeks of differentiation, cells were immunostained with the Y188 and Tuj1 (tubulin) antibodies. Note decreased APP (Y188) fluorescence, indicating APP editing. (FIG. 2C) The iPSC-derived neurons above (or isogenic APPV717I London-mutant knock-in iPSC-neurons) were transduced and differentiated as above and immunoblotted with C- and N-terminus antibodies (GSI was added to allow detection of accumulated APP CTFs). Note attenuation of APP signal with Y188 after APP-sgRNA treatment in both wild type and isogenic APP London iPSCs (quantified on right, mean±SEM of three independent experiments, ** p<0.01, ***p<0.001, ****p<0.0001). (FIG. 2D) Media from the iPSC-derived neurons above was immunoblotted for secreted sAPPα (6E10 antibody). Note increased sAPPα in sgRNA-treated samples, indicating upregulation of the non-amyloidogenic pathway. (FIG. 2E) ELISA of media from iPSC derived neurons. Note decreased Aβ in the sgRNA-treated samples (mean±SEM of three independent experiments, ** p<0.01, ***p<0.001, ****p<0.0001). (FIG. 2F) Deep sequencing of APP C-terminus in human ESCs. Red underline marks the sgRNA target sequence and arrowhead denotes predicted cut-site. Note extensive mismatch around predicted cut-site, indicating robust editing. (FIG. 2G) Major mutated APP-loci resulting from CRISPR editing, and their frequencies. (FIG. 2H) Predicted APP translational products (post-editing) for the major mutant alleles observed in deep sequencing. Note that after editing, APP is translated up-to amino acid 659 (red arrowheads; similar results were seen in HEK cells, see FIG. 6E).

FIGS. 3A-3G show the effect of APP C-terminus editing on neuronal physiology. (FIG. 3A) AAV9-sgRNA and AAV9-Cas9 expression vectors. Note that the sgRNA vector co-expresses GFP and the Cas9 is tagged to HA, for identification of transduced neurons. (FIG. 3B) Cultured hippocampal neurons were transduced with AAV9s carrying APP-sgRNA/Cas9 (or Cas9 only) and immunoblotted with the Y188 and 22C11 antibodies (in the presence of GSI). Note attenuation of CTFs by the APP-sgRNA. (FIG. 3C) Neurons were transfected (at the time of plating) with a vector expressing APP-sgRNA and Cas9. Neuritic/axon outgrowth was analyzed after 5-6 days. Neurons were transfected or infected at DIV7 with APP CRISPR, and synapse structure/function was analyzed after 14-17 days. (FIG. 3D) Top: Representative images of neurons transfected with the APP-sgRNA/Cas9 (or Cas9 alone). Bottom: Axon length and number of neurites/branches in the APP-sgRNA/Cas9 (or Cas9 alone) groups; note that there was no significant difference (mean±SEM; axon length: 30 cells for Cas9 only and 27 cells for moAPP-sgRNA from two independent experiments, p=0.2462; neurite number: 35 cells for Cas9 only and 31 cells for moAPP-sgRNA from two independent experiments, p=0.2289; branch number: 27 cells for both conditions from two independent experiments, p=0.6008). (FIG. 3E) Neurons were infected with AAV9 viruses carrying APP-sgRNA/Cas9 (or Cas9 only as controls), and fixed/stained with the presynaptic marker VAMP2. Note that the presynaptic density (VAMP2 puncta) was similar in both groups (quantified on right, mean±SEM of VAMP2 staining along 27 dendrites for Cas9 only and 25 dendrites for moAPP-sgRNA from two independent experiments, p=0.3132). (FIG. 3F) Neurons were transfected with APP-sgRNA/Cas9 (or Cas9 only as controls). Spine density in the APP-sgRNA/Cas9 (or Cas9 only) groups was also similar, quantified on right (mean±SEM of 18 dendrites for Cas9 only and 16 dendrites for moAPP-sgRNA from two independent experiments, p=0.7456). (FIG. 3G) Miniature excitatory postsynaptic currents (mEPSC) were recorded from neurons infected with AAV9-APP-sgRNA/Cas9 or AAV9-Cas9 alone. Top: Representative mEPSC traces in control and APP-sgRNA transduced neurons. Corresponding alignments of mEPSCs with average (white traces) are shown on right. Bottom: Cumulative histograms of mEPSC amplitude, 20-80% rise-time and inter-event interval in APP-sgRNA/Cas9 and the Cas9-only infected neurons (note no significant differences).

FIGS. 4A-4G show gene editing of APP C-terminus in vivo. (FIG. 4A) AAV9-sgRNA and AAV9-Cas9 were stereotactically co-injected into dentate gyrus of 8-week old mouse brains (bottom). Two weeks after viral delivery, brains were perfused, fixed, and immunostained with anti-GFP, anti-HA and anti-APP(Y188) antibodies. (FIG. 4B) Co-expression of AAV9-sgRNA-GFP and AAV9-HA-Cas9 in the dentate gyrus. Note that majority of neurons are positive for both GFP and HA (˜87% of the cells were positive for both; sampling from 3 brains). (FIGS. 4C-4D) Coronal section of a mouse hippocampi injected on one side (marked by arrow) with the AAV viruses as described above. Note attenuated Y188 staining of neurons on the injected side, indicating APP-editing. The image of mouse hippocampus injected with Cas9 only is not shown. Fluorescence quantified in (d), mean±SEM, data from three brains. One-way ANOVA: p<0.0001. Tukey's multiple comparisons: p=0.4525 (Un-injected vs Cas9 only); p<0.0001 (Un-injected vs APP-sgRNA); p<0.0001 (Cas9 only vs APP-sgRNA). (FIG. 4E) Intracerebroventrical injection of the AAV9 viruses into P0 pups. Note widespread delivery of gRNA into brain, as evident by GFP fluorescence. (FIG. 4F) Brain sections from above were immunostained with the Y188 antibody. Note attenuated Y188 staining in the APP-sgRNA/Cas9 transduced sample, suggesting APP-editing. (FIG. 4G) Western blots of the brains from (e). Note decreased expression of CTFs in the APP-sgRNA/Cas9 transduced brains; blots quantified on right (mean±SEM of three independent experiments, **p<0.01).

FIGS. 5A-5E show mechanistic details of CRISPR-guided APP editing. (FIG. 5A) APP/BACE-1 interaction—as evaluated by fluorescence complementation in cultured hippocampal neurons—was attenuated in neurons transfected with an APP C-terminus truncation mimicking the post-edited translational product (APP659:VN; quantified below, mean±SEM of 12 cells for APP(WT) and 13 cells for APP(659) from two independent experiments, p<0.0001). (FIG. 5B) APP β-cleavage is also attenuated in cells transfected with APP659. HEK cells were co-transfected with APPWT (or APP659) tagged to VN, and BACE-1:VC; and immunoblotted with the 6E10 antibody. Note decreased β-CTFs in cells carrying the truncated APP plasmid. (FIG. 5C) Schematic showing the CRISPR-edited C-terminus portion of APP. Note that the threonine at 668 position, and the endocytic YENPTY motif (dashed boxes) are thought to play roles in Aβ production (see text). (FIG. 5D) APP/BACE-1 interaction—as evaluated by fluorescence complementation in cultured hippocampal neurons—was most markedly attenuated in neurons transfected with mutant YENPTY (mean±SEM of 32 cells for APP(WT), 37 cells for APP(T668A), 45 cells for APP(YENPTY) and 49 cells for APP(T668A+YENPTY) from two independent experiments). One-way ANOVA: p<0.0001. Tukey's multiple comparisons: p=0.0022 (APP vs APP^{T668A}); p<0.0001 (APP vs APP^{YENPTY}); p<0.0001 (APP vs APP^{T668A+YENPTY}); p<0.0001 (APP^{T668A} vs APP^{YENPTY}); p<0.0001 (APP^{T668A} vs APP^{T668A+YENPTY}); p=0.7568 (APP^{YENPTY} vs APP^{T668A+YENPTY}). (FIG. 5E) Strategy of APP internalization assay. Neuro 2a cells are transfected with APP:GFP or APP659:GFP. After incubation with anti N-terminal APP antibody (22C11) for 10 min, the cells were fixed and stained with secondary antibody to visualize the cell surface and internalized APP. Note the cell surface accumulation and decreased internalization of APP659 (mean±SEM of 21 cells from two independent experiments, p<0.0001).

FIGS. 6A-6E show the choice of CRISPR editing site at APP C-terminus. (FIG. 6A) Strategy to integrate APP:VN and BACE-1:VC into the H4 genome and generation of a stable cell line expressing single copies of the two proteins (see results and methods for details). (FIG. 6B) APP and BACE-1 expression in the H4^{single copy} cell line. Note negligible expression of endogenous proteins in native H4 cells. (FIG. 6C) The H4^{single copy} cell line was transduced with lentiviral vectors carrying non-targeting control-sgRNA/Cas9 or various human APP C-terminus targeting sgRNAs/Cas9 (see Table 5 for targeting sequences). The APP/BACE-1 Venus complementation was visualized by fluorescence microscopy. Note attenuation of complementation, indicating editing by the APP-sgRNAs (quantified on right, mean±SEM of three independent experiments). One-way ANOVA: p<0.0001. Tukey's multiple comparisons: p<0.0001 (control-sgRNA vs APP659-sgRNA); p<0.0001 (control-sgRNA vs APP670-sgRNA); p<0.0001 (control-sgRNA vs APP676-sgRNA); p=0.0064 (APP659-sgRNA vs APP670-sgRNA); p=0.0015 (APP659-sgRNA vs APP676-sgRNA); p=0.6207 (APP670-sgRNA vs APP676-sgRNA). (FIG. 6D) ELISA of media from the H4^{single copy} cell line (treated as above). Note decreased Aβ in the APP-sgRNAs treated samples (mean±SEM of three independent experiments). One-way ANOVA for Aβ 40 and 42: p<0.0001. Tukey's multiple comparisons for Aβ 40: p<0.0001 (control-sgRNA vs APP659-sgRNA; control-sgRNA vs APP670-sgRNA; control-sgRNA vs APP676-sgRNA); p=0.0331 (APP659-sgRNA vs APP670-sgRNA); p=0.0071 (APP659-sgRNA vs APP676-sgRNA); p=0.6673 (APP670-sgRNA vs APP676-sgRNA). Tukey's multiple comparisons for Aβ 42: p<0.0001 (control-sgRNA vs APP659-sgRNA; control-sgRNA vs APP670-sgRNA; control-sgRNA vs APP676-sgRNA); p=0.0068 (APP659-sgRNA vs APP670-sgRNA); p=0.0221 (APP659-sgRNA vs APP676-sgRNA); p=0.8079 (APP670-sgRNA vs APP676-sgRNA). (FIG. 6E) HEK cells were transduced by lentiviral vectors carrying APP-sgRNAs and Cas9 (or non-targeting control-sgRNA/Cas9 as control), and APP C-terminus was sequenced. Left: Deep sequencing of APP659-sgRNA treated cells, and Sanger sequencing followed by ICE analyses for APP670-sgRNA and APP676-sgRNA treated cells. Red underlines mark the sgRNA-targeting sequences and arrowheads denote predicted cut-sites. Right: Predicted APP translational products after CRISPR/Cas9 editing in human HEK cells for the major mutant alleles observed in sequencing analyses. Red arrowheads indicate the amino acids where APP genes were translated up to after editing.

FIGS. 7A-7D show evaluation of CRISPR editing by immunoblotting in mouse Neuro2a cells. (FIG. 7A) Neuro2a cells were co-transfected with a sgRNA that knocked out the entire APP gene and Cas9 (see Table 5 for APP targeting sequence), and immunostained with APP N-terminal and C-terminal antibodies (after 5 days in culture). Note attenuation of staining for both Y188 and 22C11. (FIG. 7B) Neuro2a cells were transfected with various APP C-terminus targeting sgRNAs (or non-targeting control-sgRNA), and immunostained with APP N-terminal and C-terminal antibodies (after 5 days in culture in the presence of GSI). Note attenuation of staining by Y188 but not 22C11, indicating selective editing of the APP C-terminus. (FIG. 7C) Neuro2A cells were transduced by lentiviral vectors carrying APP-sgRNA and Cas9 (or non-targeting control-sgRNA/Cas9 as control) and immunoblotted with the APP antibodies CT20 and M3.2 (CT20 recognizes last 20 aa; M3.2 recognizes an extracellular domain located upstream of the CRISPR/Cas9 targeting site). A GSI was added to allow detection of accumulated APP CTF's. Note attenuated signal with CT20− but not M3.2-antibody, indicating selective editing of the APP C-terminus. (FIG. 7D) Post-editing translational products in mouse (neuro 2a) cells. Note effective truncation of APP at aa 659.

FIGS. 8A-8G show APP C-terminus editing by CRISPR/Cas9. (FIG. 8A) HEK cells were transfected with human-specific APP-sgRNA and Cas9 (or Cas9 only), and immunostained with the Y188 antibody (after 5 days in culture). Note attenuation of staining, quantified on right (mean±SEM of 25 cells for Cas9 only and 43 cells for huAPP-sgRNA from two independent experiments, p<0.0001). (FIG. 8B) HEK cells were transduced by lentiviral vectors carrying APP-sgRNA and Cas9 (or non-targeting control-sgRNA/Cas9 as control) and immunoblotted with the Y188 and 22C11 antibodies (in the presence of GSI). Note attenuation of APP-CTFs in APP-sgRNA treated cells, indicating CRISPR-editing (mean±SEM of three independent experiments, p<0.0001). (FIG. 8C) HEK cells above were immunoblotted with CT20 and 2E9 antibodies (CT20 recognizes last 20 aa; 2E9 recognizes APP extracellular domain upstream of the CRISPR/Cas9 targeting site). Note attenuated signal with CT20- but not 2E9-antibody, indicating selective editing of the APP C-terminus. (FIGS. 8D-8E) Human ESCs were transduced by lentiviral vectors carrying human APP-sgRNA/Cas9 (or non-targeting sgRNA/Cas9). Samples were immunostained with the Y188 antibody (d) or immunoblotted with the Y188 and 22C11 antibodies (e). Note attenuation of APP-CTFs in sgRNA-transduced group (for immunostaining, mean±SEM of 17 colonies for control-sgRNA and 20 colonies for huAPP-sgRNA from two independent experiments, p<0.0001; for western blotting, mean±SEM of three independent experiments, p=0.001 for total APP and p<0.0001 for CTFs). (FIG. 8F) Media from iPSC derived neurons were immunoblotted for extracellular sAPPβ (in the absence of GSI). Note decrease in APP β-cleavage in the APP-sgRNA treated samples. (FIG. 8G) Media from H4^{single-copy} cells were immunoblotted for extracellular sAPPα with 6E10 antibody and sAPPβ (in the absence of GSI). Note enhanced APP α-cleavage and attenuated APP β-cleavage in the APP-sgRNA treated samples.

FIGS. 9A-9C show gene editing by APP-sgRNA likely does not influence APP γ-cleavage. (FIG. 9A) Strategy to evaluate γ-cleavage of post-edited APP. Neuro2a cells were transfected with either full length (FL) C99, or C99 truncated at aa 659 (to mimic the post-editing translational product; all constructs were GFP-tagged to confirm expression). γ-cleavage of the FL and 659 C99 was evaluated by western blotting (note that neuro2a cells have all components of the γ-secretase complex). (FIG. 9B) Schematic showing expected C99-cleavage patterns. Note that upon γ-cleavage, both C99-fragments will be further truncated. However, if the ‘CRISPR-mimic’ (659) fragment did not undergo γ-cleavage, this truncation would not occur. (FIG. 9C) Western blotting of the cells from (a) indicates that both C99 fragments (FL and 659) undergo γ-cleavage—as indicated by the shift upon inhibiting γ-cleavage by GSI. These data suggest that gene editing by the APP-gRNA likely does not affect APP γ-cleavage, and that the effects seen on the amyloid pathway are likely due to modulation of APP-β-cleavage.

FIGS. 10A-10G show off target analyses of APP-sgRNA. (FIG. 10A) Computationally predicted top five off-target (OT) sites in the genome, that can be potentially targeted by the mouse and human APP-sgRNAs (mismatched nucleotides in the targeting sequence are marked in red). Genomic locations corresponding to the sequences is shown on the right column (note most are in non-coding regions). (FIG. 10B) Strategy of T7 endonuclease digestion assay to detect genome-editing events. Genomic DNA was PCR amplified with primers bracketing the modified locus. PCR products were then rehybridized, yielding three possible structures. Duplexes containing a mismatch were digested by T7 endonuclease I. DNA gel analysis was used to calculate targeting efficiency. Note digested fragments in the gel indicates cleavage. (FIG. 10C) Gene edits at the APP locus by the APP-sgRNA, as seen by T7 endonuclease digestion. Note two digested fragments were recognized after T7 endonuclease digestion. (FIGS. 10D-10E) T7 endonuclease assays of potential off-target sites (mouse and human). No digested fragments are seen, indicating that the sgRNAs do not generate detectable gene edits at these sites. (FIG. 10F) Comparison of APLP1 and 2 sequences with APP at the sgRNA targeting site. Asterisks mark conserved nucleotide sequences, and the PAM sites are underlined. Nucleotide mis-matches are highlighted in yellow. Note extensive mis-match of the mouse and human sequences at the sgRNA targeting site. (FIG. 10G) Left: Off-target TIDE analysis of APP family members APLP1 and 2 in mouse (neuro 2a) and human (HEK) cell lines following lentiviral integration of Cas9 using TIDE. No modifications were detected below the TIDE limit of detection (dotted line) in either of the populations, indicating that the APP-gRNA was unable to edit APLP 1/2. Right: TIDE analysis of APLP1 and 2 loci in mouse and human cell lines. Neither of the populations had significant editing at either of the two loci, and all sequences had a near perfect correlation to the model.

FIGS. 11A-11C show trafficking of vesicles carrying APP(WT) or APP(659). (FIG. 11A) Cultured hippocampal neurons were transfected with APP(WT):GFP or APP(659):GFP, and kinetics of APP particles were imaged live in axons and dendrites. (FIG. 11B) Representative kymographs and quantification of APP kinetics in axons. Note that there was no change in frequency of transport, and only a modest reduction in run-length and velocity. Error bars, mean±SEM of 325 APP(WT):GFP and 310 APP(659):GFP vesicles in 10-12 neurons from two independent experiments. Frequency: p=0.4635 (APP_antero vs APP659_antero); p=0.6650 (APP_retro vs APP659_retro); p=0.7420 (APP_stat vs APP659_stat). Velocity: P<0.0001 (APP_antero vs APP659_antero); p=0.9419 (APP_retro vs APP659_retro). Run length: p<0.0001 (APP_antero vs APP659_antero); p=0.2433 (APP_retro vs APP659_retro). (FIG. 11C) Representative kymographs and quantification of APP kinetics in dendrites. Error bars, mean±SEM of 130 APP(WT):GFP and 115 APP(659):GFP particles in 10-12 neurons from two independent experiments. Frequency: p=0.3245 (APP_antero vs APP659_antero); p=0.5438 (APP_retro vs APP659_retro); p=0.2394 (APP_stat vs APP659_stat). Velocity: p=0.0120 (APP_antero vs APP659_antero); p=0.6248 (APP_retro vs APP659_retro). Run length: p=0.1352 (APP_antero vs APP659_antero); p=0.4284 (APP_retro vs APP659_retro).

FIGS. 12A-12C show internalization of APP-659-GG (most common post-editing translational product). (FIGS. 12A-12B) Neuro2a cells were co-transfected with untagged APP-659-GG and mCherry (or untagged WT APP and mCherry as control). After incubation with anti N-terminal APP antibody (22C11) for 10 min, the cells were fixed and stained with secondary antibody to visualize surface and internalized APP (mCherry labels transfected cells). Note accumulation of APP-659-GG on the cell surface, along with decreased internalization; quantified in FIG. 12B. Mean±SEM of 25 cells for APP(WT) and 26 cells for APP-659-GG from two independent experiments, p<0.0001. (FIG. 12C) Expression levels of exogenous APP constructs. Note that WT and APP-659-GG were expressed at similar levels in the Neuro2a cells above.

FIG. 13 shows the sequence of human APP (nucleotide sequence SEQ ID NO:11, amino acid sequence SEQ ID NO:12) and mouse APP (nucleotide sequence SEQ ID NO:13, amino acid sequence SEQ ID NO:14) along with the corresponding sequences of the gRNA used in select gene editing embodiments described herein.

FIG. 14 shows the sequence of the Cas9 nuclease gene sequence.

FIG. 15 shows the sequence and vector map of an exemplary vector (SEQ ID NO:17) for APP truncation at amino acid 659. The vector includes the gRNA sequence (lowercase italics) and the Cas9 nuclease sequence.

FIG. 16 shows the sequence and vector map of an exemplary Cas9 vector (SEQ ID NO:18).

FIG. 17 shows the sequence and vector map of an exemplary APP sgRNA vector (SEQ ID NO:19).

FIG. 18 shows the sequence and vector map of an exemplary lentiviral APP sgRNA vector (SEQ ID NO:20).

### INCORPORATION BY REFERENCE

All publications, including but not limited to patents and patent applications, cited in this specification are herein incorporated by reference as though set forth in their entirety in the present application.

### DETAILED DESCRIPTION OF THE INVENTION

### In General

Gene-editing methods, such as CRISPR/Cas9 guided gene-editing, hold promise as a therapeutic tool. However, few studies have applied the technology to neurodegenerative diseases. Moreover, the conventional approach of mutation-correction is limited in scope to inherited diseases which are a small fraction of neurodegenerative disease cases. The present invention introduces a strategy to edit endogenous amyloid precursor protein (APP) at the extreme C-terminus and selectively attenuate the amyloidogenic pathway—a key pathologic cascade in Alzheimer's disease (AD). In the method of the present invention, the APP N-terminus remains intact and protective a-cleavage is up-regulated.

The Examples below demonstrate that robust APP-editing is demonstrated in cell lines, human stem cells, cultured neurons, and in mouse brains. Physiologic parameters remain unaffected. Without being bound by any particular theory, the present invention works by restricting the physical interaction of APP and BACE-1, said interaction being the rate-limiting step in amyloid-β (Aβ) production. The Examples below delineate underlying mechanisms that abrogate APP/BACE-1 interaction in this setting. The invention offers an innovative ‘cut and silence’ gene-editing strategy that could be a new therapeutic paradigm for AD.

CRISPR/Cas9 works by inducing sequence-specific double-stranded breaks (DSBs) in DNA. After such breaks, the cell undergoes an error-prone repair process called non-homologous end joining, leading to a disruption in the translational reading frame, often resulting in frameshift mutations and premature stop codons. For the system to work, at least two components must be introduced in cells: a Cas9 nuclease and a guide RNA. Described herein are CRISPR/Cas9 constructs suitable for truncation of the APP protein and disruption of amyloid-β production.

### CONSTRUCTS OF THE PRESENT INVENTION

In a first aspect, the present invention provides a construct for CRISPR mediated cleavage of the APP gene. The constructs of the present invention include a nucleotide sequence encoding a Cas9 nuclease and a guide RNA (gRNA). In some embodiments the sequence encoding the Cas9 nuclease and the gRNA are included on a single vector construct. In some embodiments the sequence encoding the Cas9 nuclease is included in a vector construct separate from a vector construct encoding for the gRNA. Additionally, the construct may include a promoter, a poly(A) tail, an optional reporter element, and an optional selection marker such as an ampicillin selection marker.

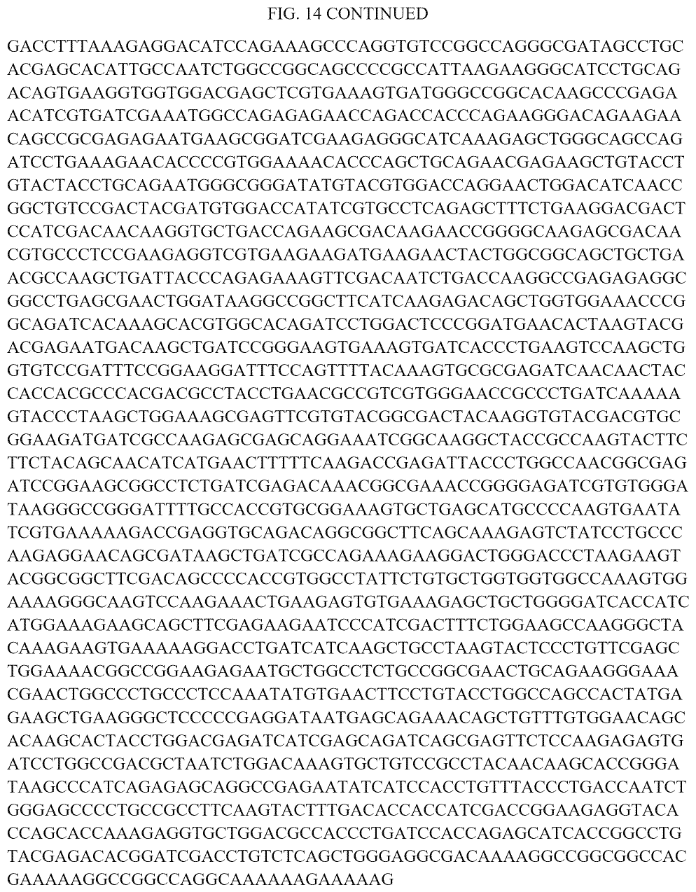

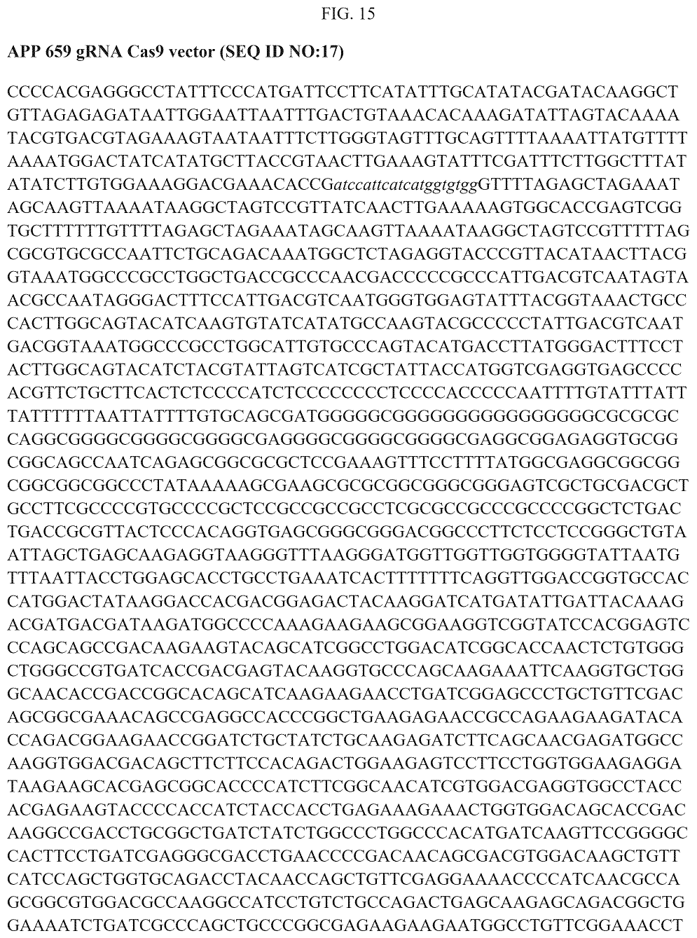

As used herein “Cas9 nuclease” refers to the RNA-guided DNA endonuclease enzyme associated with the CRISPR adaptive immunity system in Streptococcus pyogenes and other bacteria. The Cas9 nuclease includes two nuclease domains, a RuvC-like nuclease domain located at the amino terminus, and a HNH-like nuclease domain. In some embodiments, the sequence of the Cas9 nuclease is the sequence included in FIG. 14 (SEQ ID NO:15).

In some embodiments, the Cas nuclease is expressed under the control of a neuron specific promoter or ubiquitous promoter. The neuron specific promoter may be any neuron specific promoter known in the art (see for example, Swiech L et al., In vivo interrogation of gene function in the mammalian brain using CRISPR-Cas9. Nature Biotechnology 2015 January; 33(1): 102-6). In some embodiments the neuron specific promoter is the human synapsin 1 (hSyn1) promoter. In some embodiments the neuron specific promoter is the mouse Mecp2 promoter (pMecp2). In some embodiments the ubiquitous promoter is the chicken 3-actin promoter. In some embodiments, the ubiquitous promoter is an EFS promoter.

In one embodiment of the present invention, the construct is specific for the extreme C-terminus of the APP gene. By “APP gene” or “amyloid precursor protein”, we mean to include the human APP gene as disclosed in Hendricks et al (Hendriks L et al. Presenile dementia and cerebral haemorrhage linked to a mutation at codon 692 of the beta-amyloid precursor protein gene. Nature Genetics 1992 June; 1(3): 218-21) and recited herein as SEQ ID NO:11. The amino acid sequence of the APP gene is recited as SEQ ID NO:12.

As used herein “extreme C-terminus,” refers of a portion of the C-terminus of the APP protein which, when absent, is sufficient to disrupt the interaction between APP and BACE. The truncated APP lacking the extreme C-terminus will still include its native N-terminus, the transmembrane domain and the residual C-terminal region. Typically, the extreme C-terminus of the APP protein will mean 8 or more amino acids at the C-terminus of the APP protein. This may be accomplished by CRISPR/Cas9 mediated cleavage of the APP gene such that the expressed APP protein is truncated to a length selected from the group consisting of 659, 670, 676, or 686, relative to SEQ ID NO:12 (human) or SEQ ID NO:14 (mouse). In some embodiments, the APP gene is cleaved following a nucleotide selected from the group consisting of 1978, 2009, 2010, 2029, and 2058 relative to SEQ ID NO:1 (human) or SEQ ID NO:14 (mouse). A list of these cleavage sites is included in the table below.

As used herein “guide RNA (gRNA)” refers to the 20 nucleotide target sequence which directs Cas9 mediated cleavage within the APP gene. The gRNA will be encoded on a synthetic RNA construct which additionally includes the tracrRNA sequence. While the gRNA sequence is variable and will be specific for the cleavage site of interest, the tracrRNA is the same for all gRNA sequences used. The tracrRNA sequence is SEQ ID NO:16 The gRNA described herein are specific for the truncation of the C-terminal segment of APP. Suitable target sequences within the APP gene for design of gRNA sequences are recited below, which includes the sequence of the gRNA.

tracrRNA (SEQ ID NO: 16):
5′-GTTTTAGAGCTAGAAATAGCAAGTTAAAATAAGGCTAGTC
CGTTATCAACTTGAAAAAGTGGCACCGAGTCGGTGCTTTT -3

TABLE 1
HUMAN APP (FULL LENGTH 695 AA)

| | | SEQ | Position | Cas9 cleavage | Length of |
|---|---|---|---|---|---|
| sgRNA | sgRNA sequence | ID | relative | between | truncated |
| name | (5′-3′) | NO: | to APP | nucleotides | protein |
| sgRNA 1 | atccattcatcatggtgtgg | 1 | 1962 - 1981 | 1978/1979 | 659 aa |
| sgRNA 2 | tggacaggtggcgctcctct | 2 | 2007 - 2026 | 2009/2010 | 670 aa |
| sgRNA 3 | ttggacaggtggcgctcctc | 3 | 2008 - 2027 | 2010/2011 | 670 aa |
| sgRNA 4 | gtagccgttctgctgcatct | 4 | 2027 - 2046 | 2029/2030 | 676 aa |
| sgRNA 5 | tgctcaaagaacttgtaggt | 5 | 2056 - 2075 | 2058/2059 | 686 aa |

TABLE 2
MOUSE APP (FULL LENGTH 695 AA)

| | | SEQ | Position | Cas9 cleavage | Length of |
|---|---|---|---|---|---|
| sgRNA | sgRNA sequence | ID | relative | between | truncated |
| name | (5′-3′) | NO: | to APP | nucleotides | protein |
| sgRNA 1 | atccatccatcatggcgtgg | 6 | 1962 - 1981 | 1978/1979 | 659 aa |
| sgRNA 2 | tggagagatggcgctcctct | 7 | 2007 - 2026 | 2009/2010 | 670 aa |
| sgRNA 3 | ttggagagatggcgctcctc | 8 | 2008 - 2027 | 2010/2011 | 670 aa |
| sgRNA 4 | atatccgttctgctgcatct | 9 | 2027 - 2046 | 2029/2030 | 676 aa |
| sgRNA 5 | tgctcaaagaacttgtaagt | 10 | 2056 - 2075 | 2058/2059 | 686 aa |

Cleavage of the APP gene will occur between the 3rd and 4th nucleotides from the PAM site associated with the target sequence in the APP gene. For the sgRNA 1, the PAM site is on the sense strand of the APP gene, the sgRNA of SEQ ID NOs:1 and 6 are complementary to the antisense strand of the APP gene, and the cleavage will occur between nucleotides 1978 and 1979 relative to SEQ ID NO:11 (human) or SEQ ID NO:14 (mouse). For sgRNA 2, 3, 4 and 5, the PAM site is on the antisense strand of the APP gene, the sgRNA of SEQ ID NOs:2-5 and 7-10 are complementary to the sense strand of the APP gene, and the cleavage site is between nucleotides 2009 and 2010 for sgRNA 2, between nucleotides 2010 and 2011 for sgRNA 3, between nucleotides 2029 and 2030 for sgRNA 4 and between nucleotides 2058 and 2059 for sgRNA 5, relative to SEQ ID NO:11 (human) or SEQ ID NO:14 (mouse).

In some embodiments, the gRNA or tracrRNA is modified by any means known in the art. Common methods for gRNA or tracrRNA modification include chemical modifications or modifications to axillary sequences appended to the RNA to increase efficiency known in the art.

In some embodiments, the gRNA is expressed under the control of an RNA Pol III promoter. Examples of RNA Pol III promoters include, but are not limited to, U6 and H1 promoters. A promoter, generally, is a region of nucleic acid that initiates transcription of a nucleic acid encoding a product. A promoter may be located upstream (e.g., 0 bp to −100 bp, −30 bp, −75 bp, or −90 bp) from the transcriptional start site of a nucleic acid encoding a product, or a transcription start site may be located within a promoter. A promoter may have a length of 100-1000 nucleotide base pairs, or 50-2000 nucleotide base pairs. In some embodiments, promoters have a length of at least 2 kilobases (e.g., 2-5 kb, 2-4 kb, or 2-3 kb).

In some embodiments, the construct comprises an optional reporter element. The reporter element may be any reporter known in the art including, but not limited to, mCherry, green fluorescent protein, and human influenza hemagglutinin (HA).

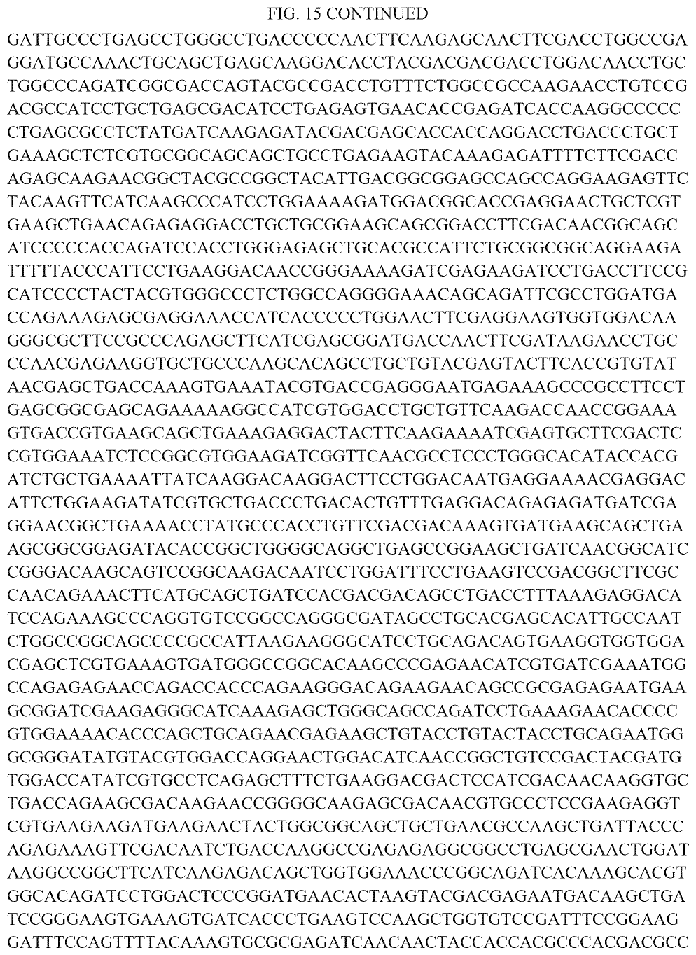

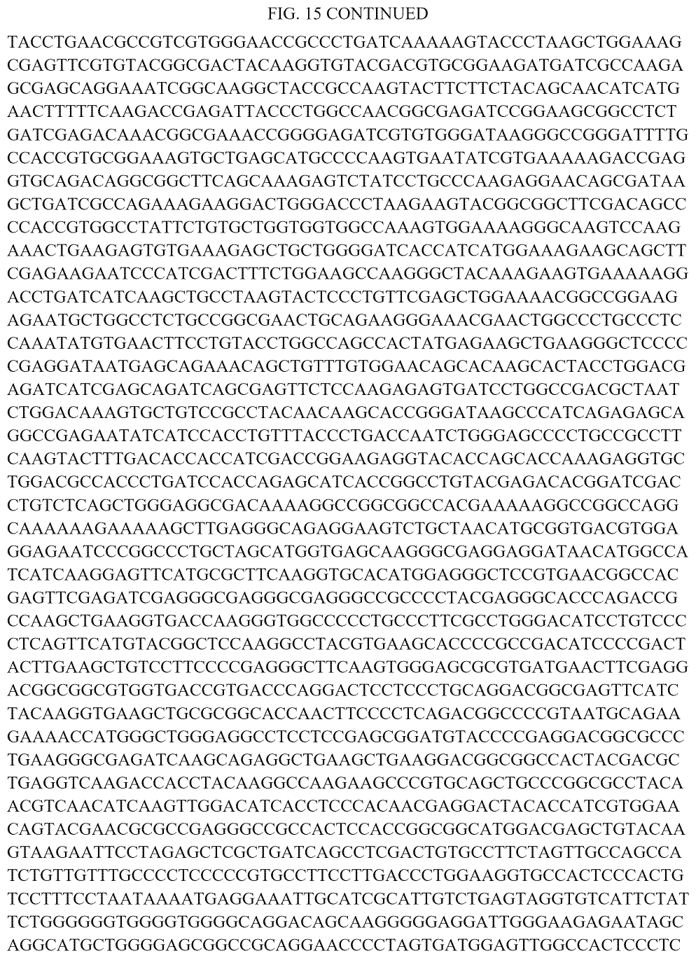

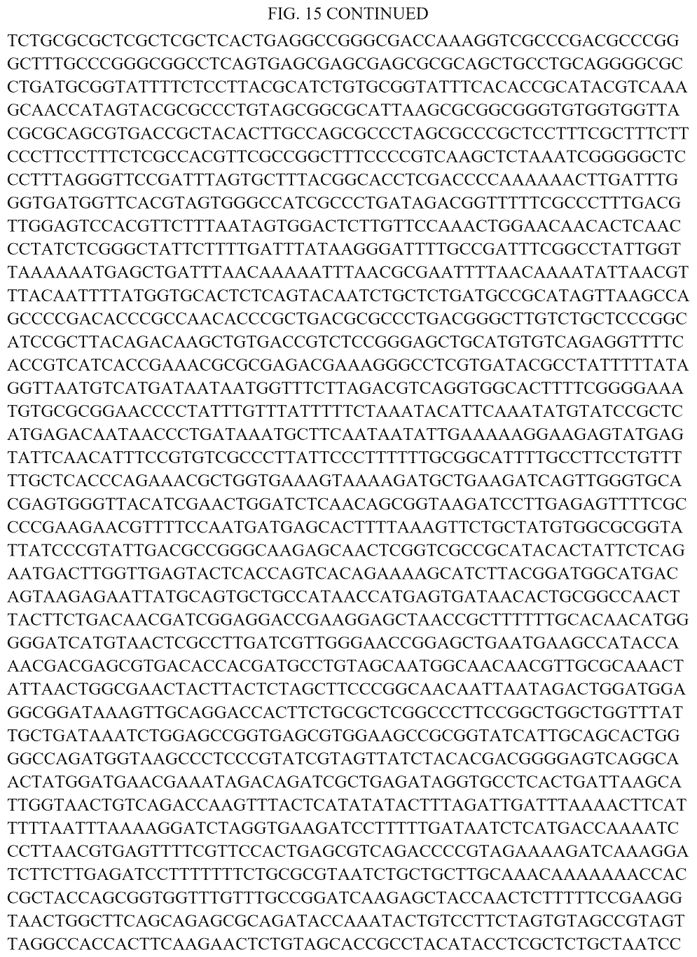

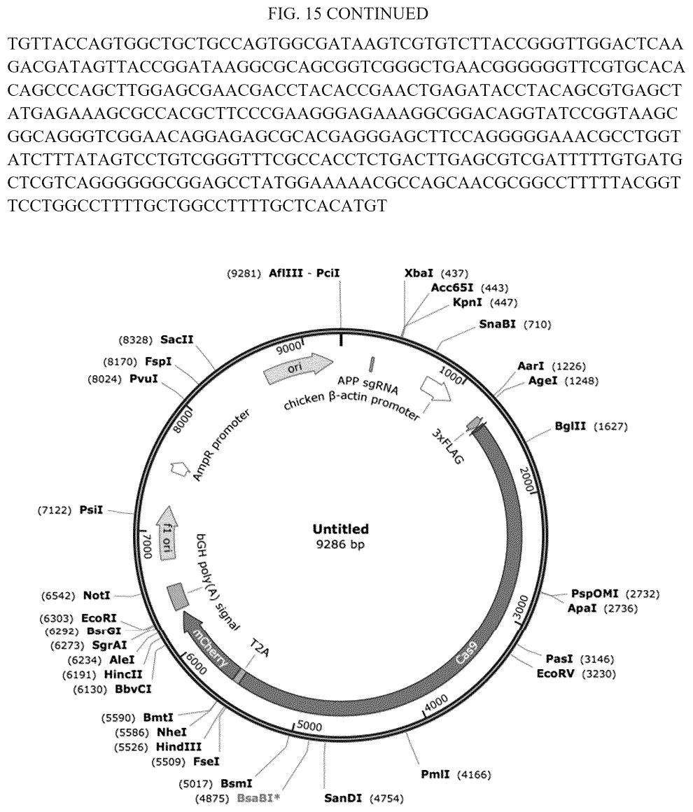

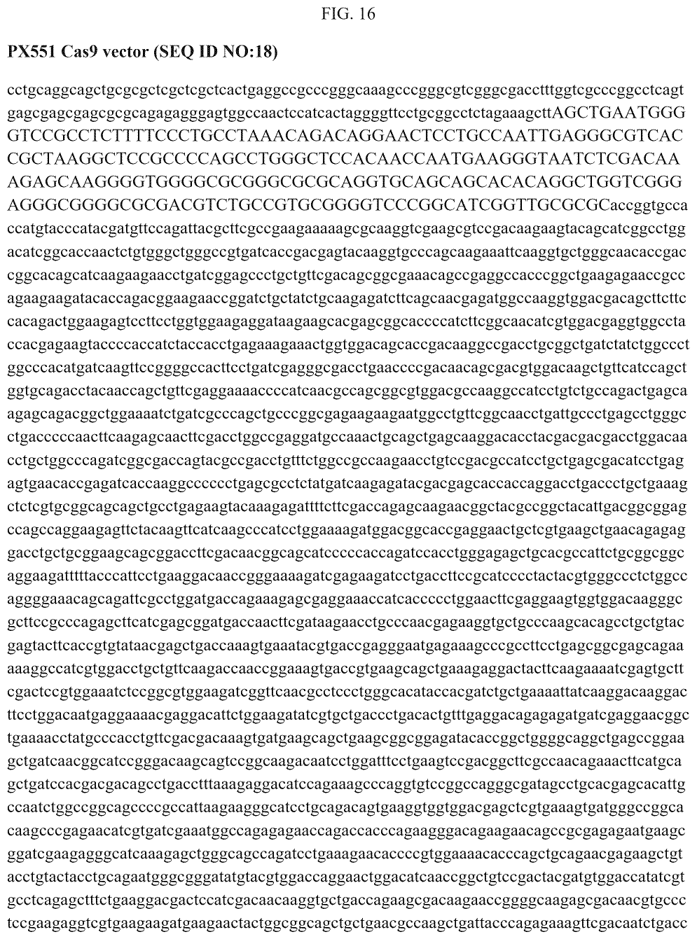

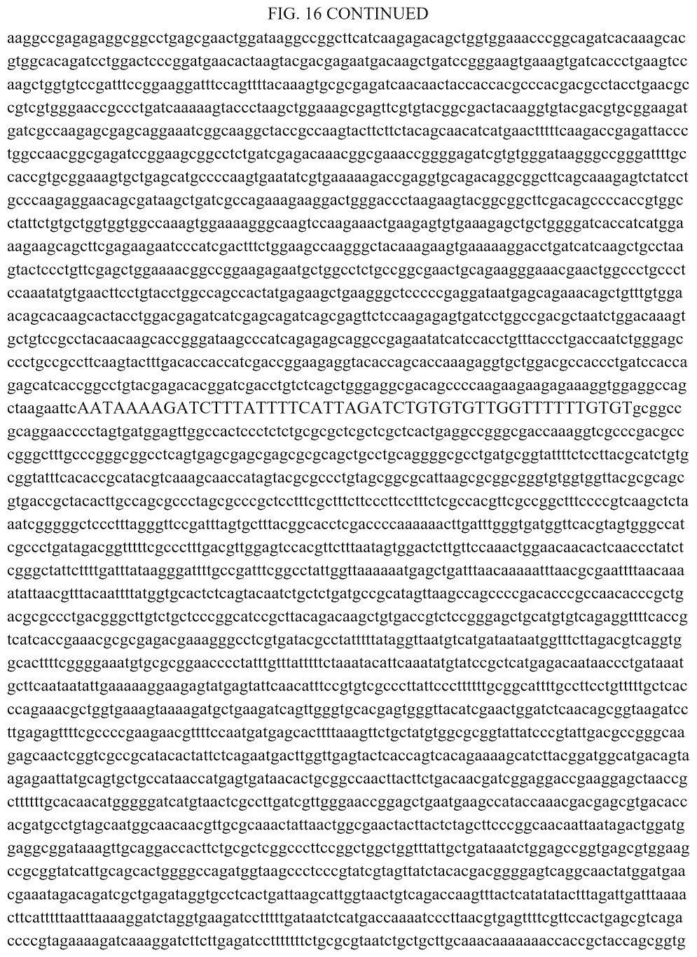

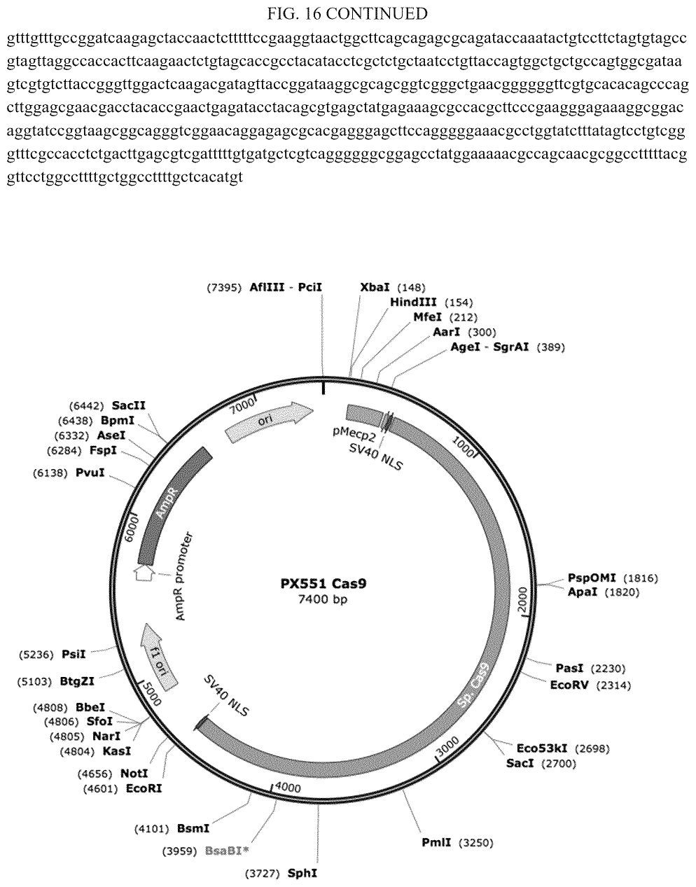

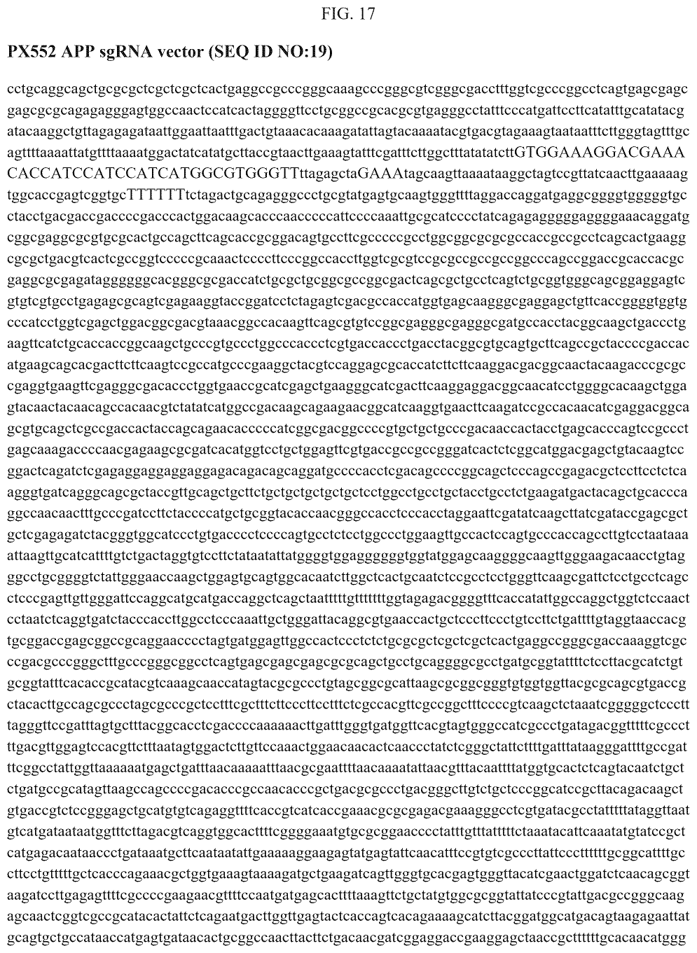

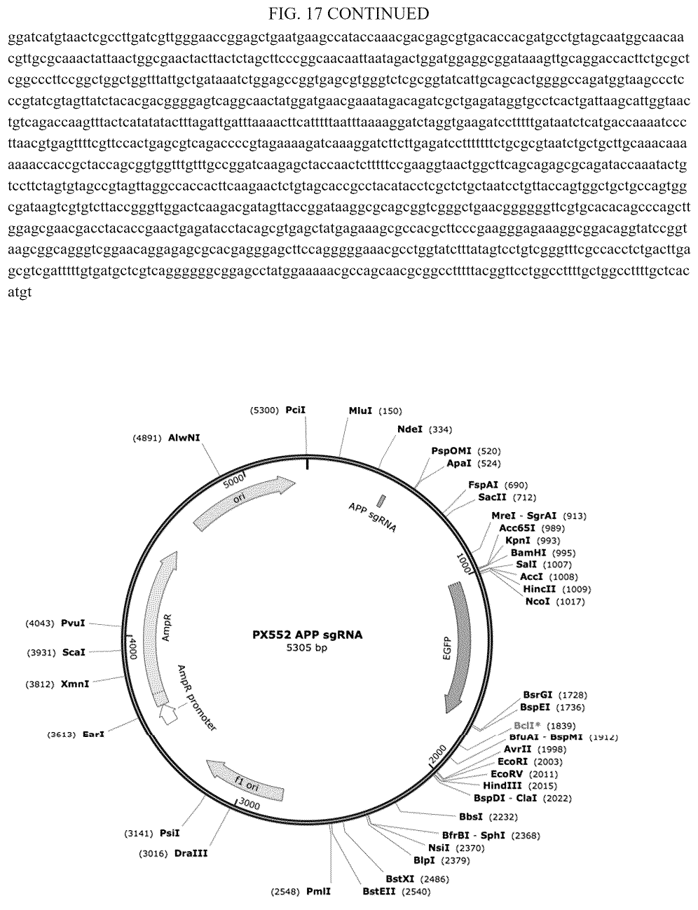

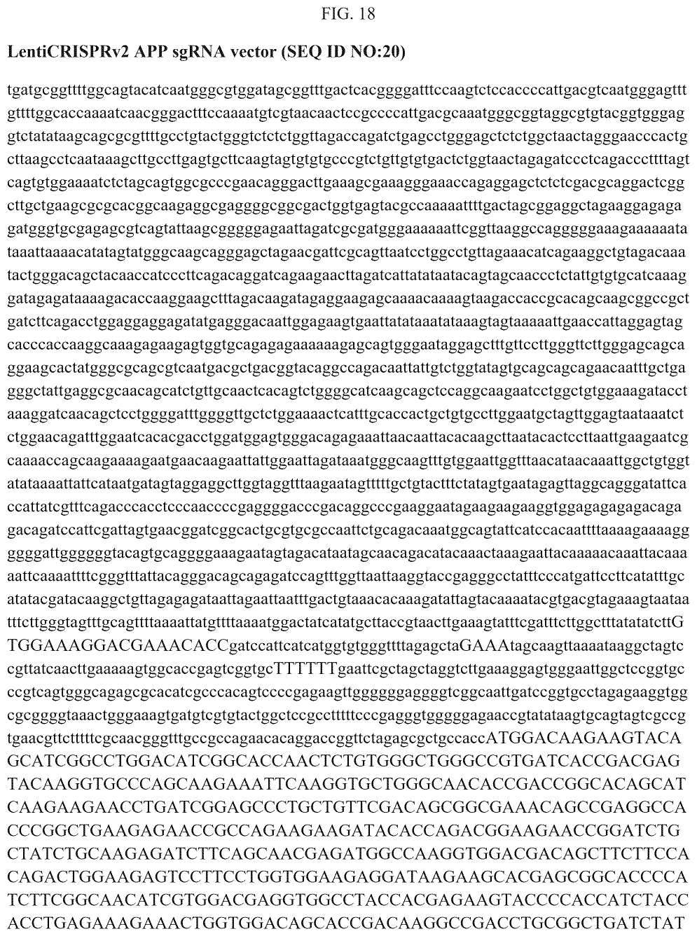

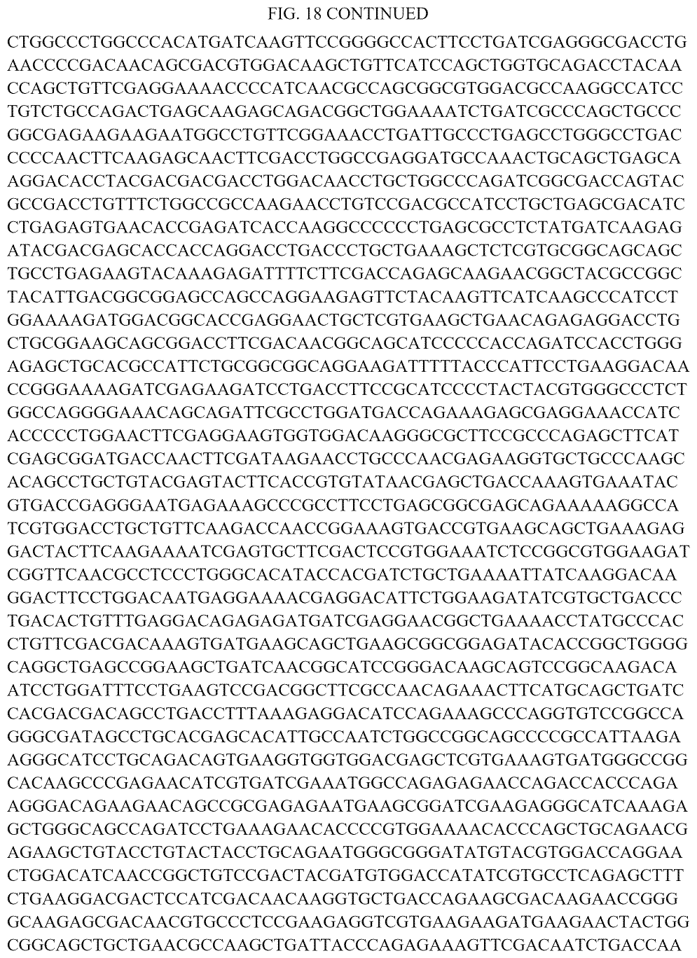

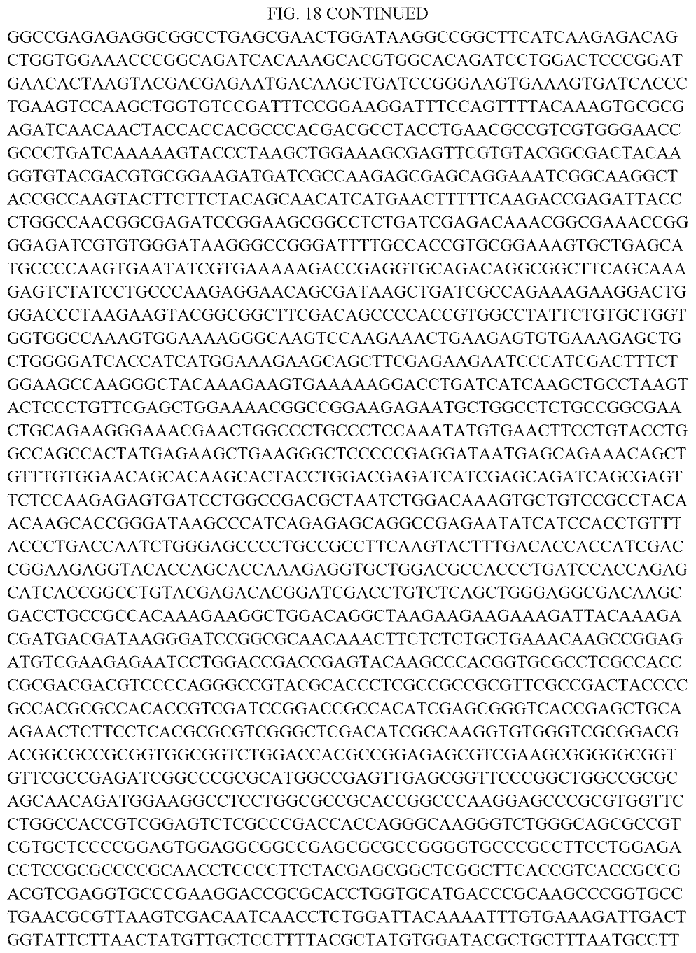

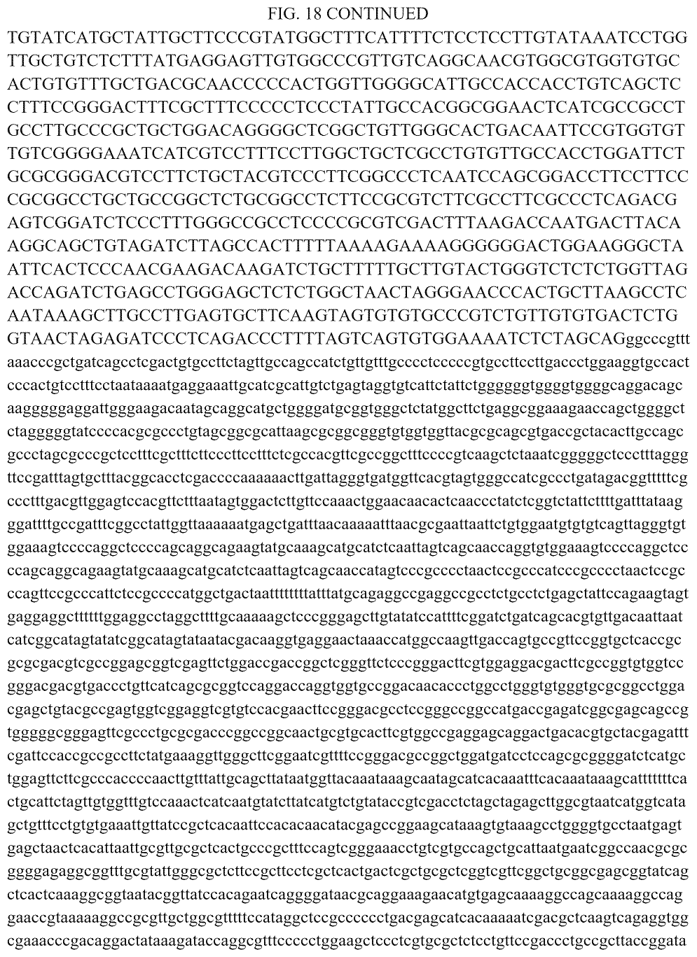

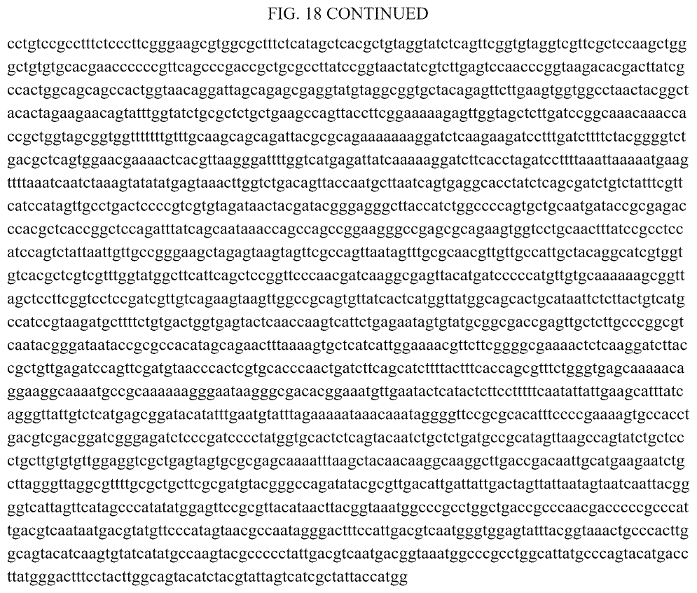

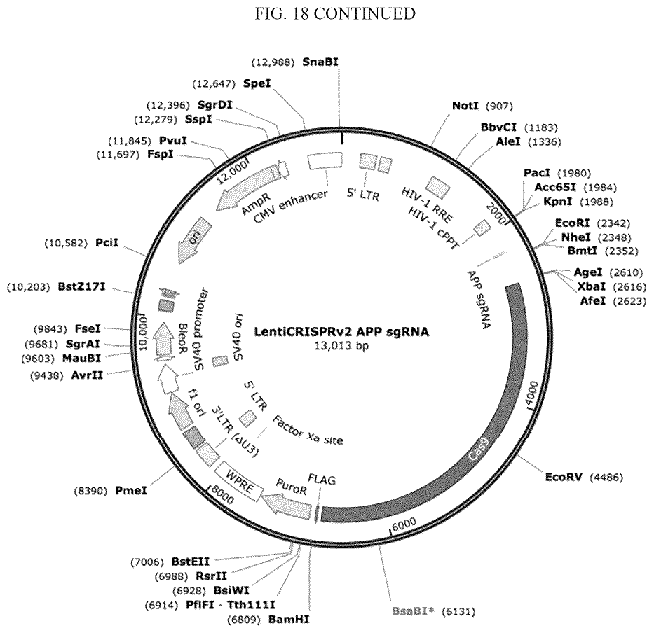

In some embodiments, the constructs are packaged in a vector suitable for delivery into a mammalian cell, including but not limited to, an adeno-associated viral (AAV) vector, a lentiviral vector, or a vector suitable for transient transfection. Suitable vector backbones are known and commercially available in the art. For example, see Deverman et al. (Cre-dependent selection yields AAV variants for widespread gene transfer to the adult brain, Nature Biotechnology, 34(2):204-209, 2016) and Chan et al. (Engineered AAVs for efficient noninvasive gene delivery to the central and peripheral nervous system, Nature Neuroscience, 20(8):1172-1179, 2017) which are incorporated herein by reference in their entirety. In some embodiments, the vector is an AAV vector and the gRNA and Cas9 constructs are encoded on separate vectors. In some embodiments, the vector is a lentiviral vector and the gRNA and Cas9 constructs are encoded on a single vector. In some embodiments, the vector is a vector suitable for transient transfection and the gRNA and Cas9 constructs are encoded on a single vector. In one embodiment the vector includes the sequence of SEQ ID NO:17. In some embodiment, the gRNA and Cas9 constructs are encoded on separate AAV vectors wherein the gRNA is encoded on a vector comprising SEQ ID NO:19 and the Cas9 construct is encoded on a vector comprising SEQ ID NO:18. In some embodiments, the vector is a lentiviral vector and comprises the sequence of SEQ ID NO:20. The vectors of SEQ ID NOs:17-20 are included in FIGS. 15-18.

In some embodiments, the vectors encoding the constructs described herein may optionally include a monoclonal antibody tag (e.g., FLAG), one or more origins of replication (e.g., fl ori), one or more terminator sequences (e.g., bGH), one or more polyadenylation tags (bGH poly(A)), and one or more inverted terminal repeats (ITR). The vector may also include one or more selectable markers, such as an antibacterial resistance marker such as an ampicillin selectable marker. A skilled artisan will be familiar with the elements and configurations necessary for vector construction to encode the constructs described herein.

### METHODS OF THE PRESENT INVENTION

The constructs described herein may be formulated with a pharmaceutically acceptable carrier for administration to a patient in need thereof. A pharmaceutically acceptable carrier may be, but is not limited to, a nanoparticle cage including the one or more vectors of the present invention.

To function as therapeutic agents, the constructs described herein are delivered into neurons in the patient's brain, crossing the blood brain barrier (BBB). In one embodiment, one would attach or associate the CRISPR components with a delivery system, such as a nanoparticle delivery system. In some embodiments, the constructs are formulated using an AAV vector and are delivered intravenously. In some embodiments, the constructs are delivered intrathecally into the spinal fluid of the patient. In some embodiments, the constructs are delivered directly into the brain of the patient.

As used herein, the terms “treat” and “treating” refer to therapeutic measures, wherein the object is to slow down or alleviate (lessen) an undesired physiological change or pathological disorder resulting from Alzheimer's disease. For purposes of this invention, treating the disease, condition, or injury includes, without limitation, alleviating one or more clinical indications, reducing the severity of one or more clinical indications of Alzheimer's disease, diminishing the extent of the condition, stabilizing the subject's Alzheimer's disease (i.e., not worsening), delay or slowing, halting, or reversing Alzheimer's disease and bringing about partial or complete remission Alzheimer's disease. Treating Alzheimer's disease also includes prolonging survival by days, weeks, months, or years as compared to prognosis if treated according to standard medical practice not incorporating treatment with the constructs described herein.

Subjects in need of treatment can include those already having or diagnosed with Alzheimer's disease as well as those prone to, likely to develop, or suspected of having Alzheimer's disease, such as a subject with a genetic predisposition to or family history of Alzheimer's disease. Subjects in need of treatment may be those with a familial AD mutation or wild-type patients without a mutation. In some embodiments, a subject in need of treatment may be a subject who had been diagnosed by a positron emission tomography (PET) scan, a blood test or other means known in the art to have AD or to be predisposed to AD. Pre-treating or preventing Alzheimer's disease according to a method of the present invention includes initiating the administration of a therapeutic (e.g., the APP gRNA and Cas9 constructs described herein) at a time prior to the appearance or existence of the disease or injury, or prior to the exposure of a subject to factors known to induce Alzheimer's disease. Pre-treating the disorder is particularly applicable to subjects at risk of having or acquiring the disease injury.

As used herein, the terms “prevent” and “preventing” refer to prophylactic or preventive measures intended to inhibit undesirable physiological changes or the development of Alzheimer's disease. In exemplary embodiments, preventing Alzheimer's disease comprises initiating the administration of a therapeutic (e.g., the APP gRNA and Cas9 constructs described herein) at a time prior to the appearance or existence of Alzheimer's disease such that the disease, or its symptoms, pathological features, consequences, or adverse effects do not occur. In such cases, a method of the invention for preventing Alzheimer's disease comprises administering the APP gRNA and Cas9 constructs described herein to a subject in need thereof prior to the onset or development of Alzheimer's disease in a patient at risk for Alzheimer's disease such as a patient with a genetic risk factor or a patient with a family history of Alzheimer's disease.

As used herein, the terms “subject” or “patient” are used interchangeably and can encompass a human or mouse. As used herein, the phrase “in need thereof” indicates the state of the subject, wherein therapeutic or preventative measures are desirable. Such a state can include, but is not limited to, subjects having Alzheimer's disease or a pathological symptom or feature associated with Alzheimer's disease.

### EXAMPLES

The embodiment described in this example demonstrates truncation of the C-terminus of the APP protein, attenuation of APP-β-cleavage and Aβ production, and manipulation of the amyloid pathway using CRISPR/Cas9 gene editing.

A common theme in neurodegenerative diseases is that proteins normally present in the brain (APP, tau, α-synuclein, TDP-43, etc.) acquire toxic properties—or trigger pathologic cascades—that ultimately lead to synaptic loss and neurodegeneration. Our broad idea is to rationally edit small segments of endogenous proteins known to play key roles in the progression of disease, with the ultimate goal of attenuating their pathologic activity. As endogenous proteins expectedly play physiologic roles, it is also important to conserve their normal function, as far as possible. Here we show conceptual proof of this ‘selective silencing’ approach for APP. APP is a single-pass transmembrane protein, cleaved by the enzymes β- and γ-secretases to ultimately generate Aβ—a neuropathologic hallmark of AD. APP cleavage by the β-secretase BACE-1 is the rate limiting step in this ‘amyloidogenic’ pathway. Alternatively, APP is cleaved by α-secretases—the ‘non-amyloidogenic’ pathway—that is thought to be neuroprotective because it precludes β-cleavage of APP (6,7); and studies have highlighted neuroprotective effects of APP-α-cleavage products in vivo (8,9).

We recently developed a Bi-molecular fluorescence complementation (BifC) assay to visualize the physical approximation of APP and BACE-1 in neurons (10). As a control for validation, we found that a C-terminal deletion also abrogated APP/BACE-1 complementation (10); in line with previous studies showing that deletions/mutations of the APP C-terminus can attenuate Aβ production (11-13). Collectively, these observations originally gave us the idea of using CRISPR/Cas9-mediated truncation of native APP to attenuate APP-β-cleavage and Aβ production in AD. Using CRISPR-tools, cell/molecular biology, live imaging, deep sequencing, electrophysiology and in vivo animal studies, here we highlight a strategy to favorably manipulate the amyloid pathway by gene editing.

### Results and Discussion

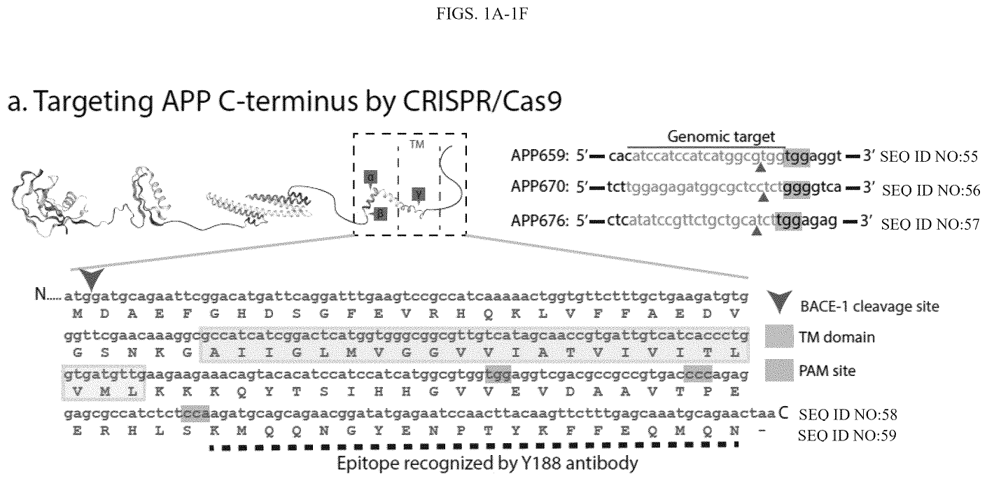

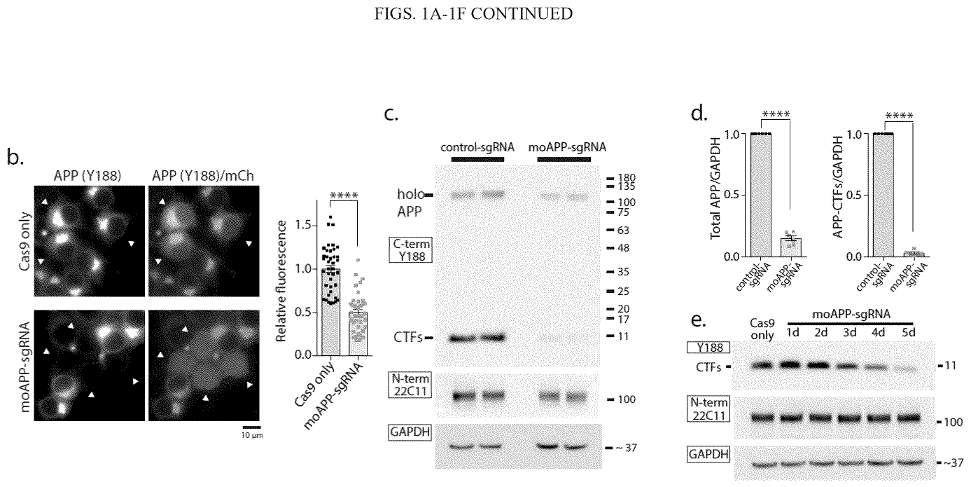

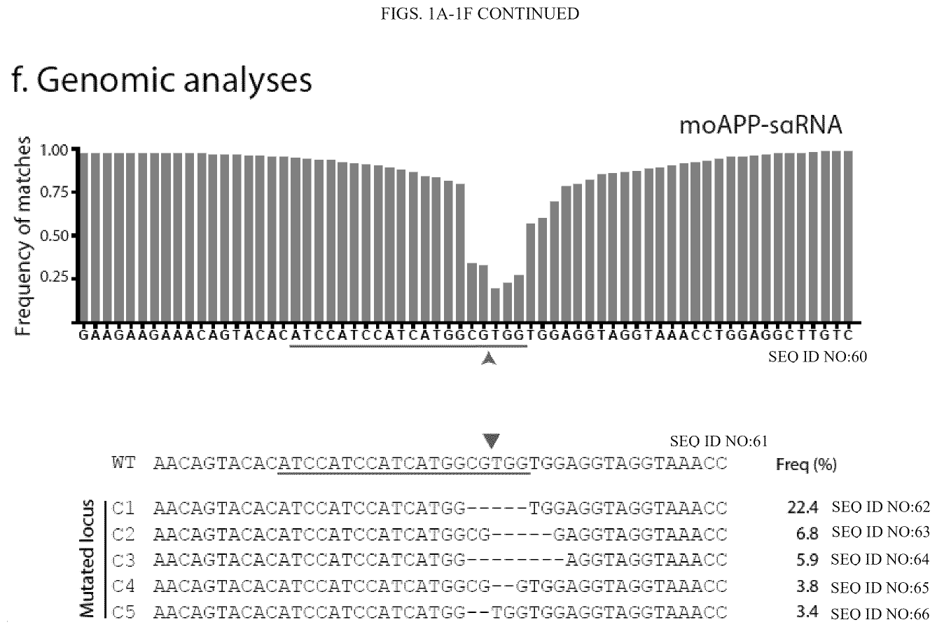

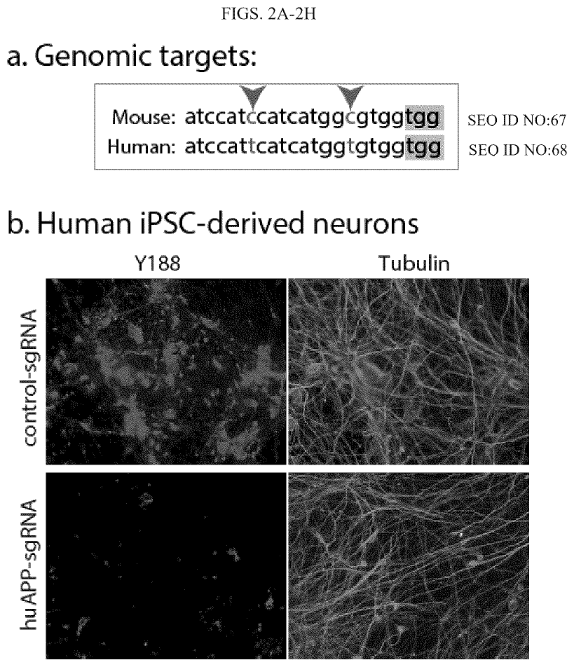

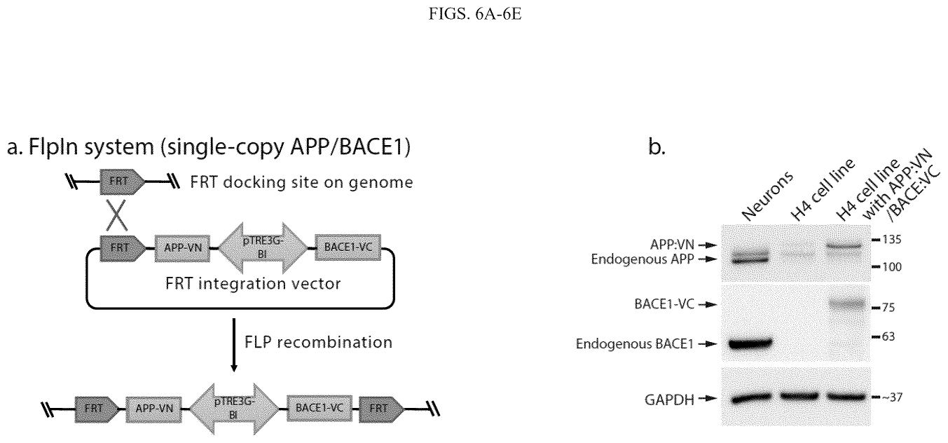

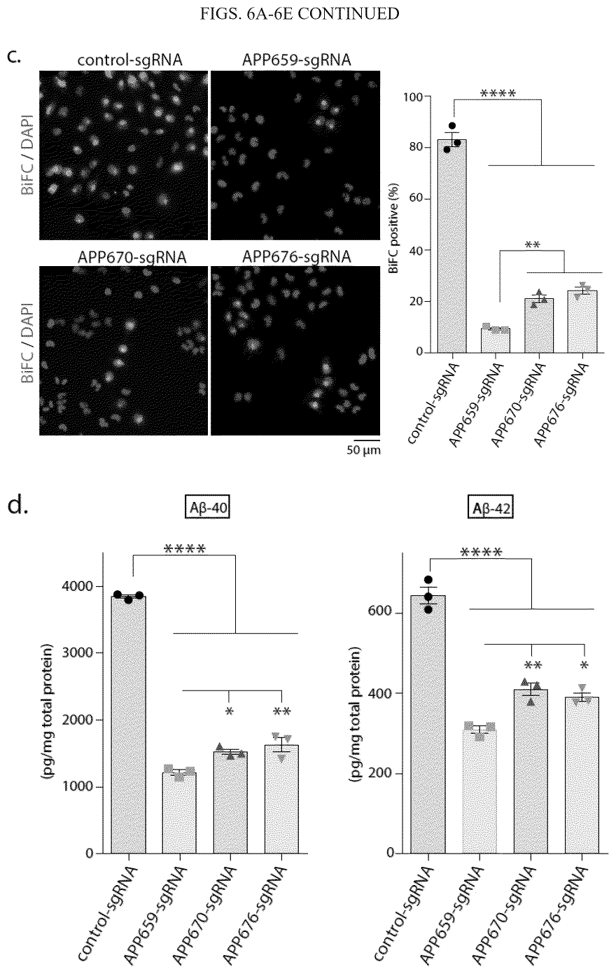

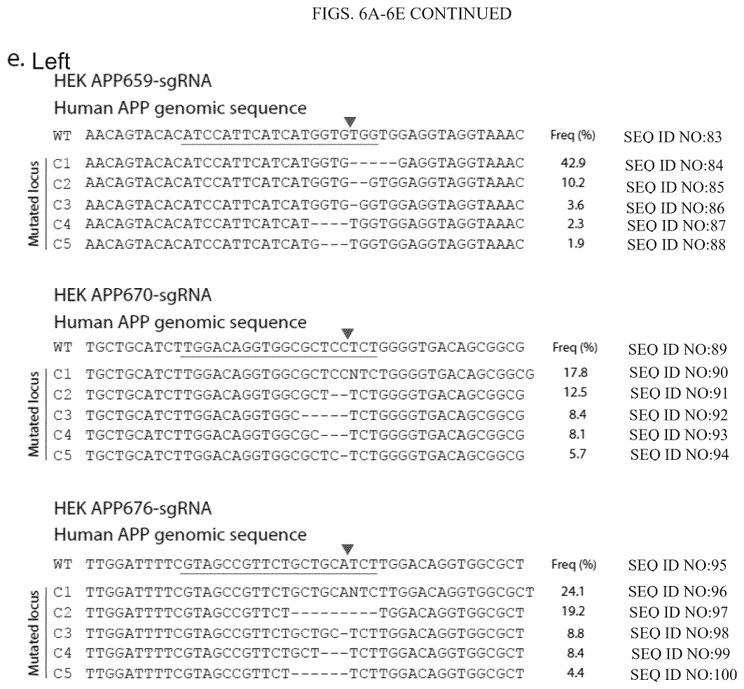

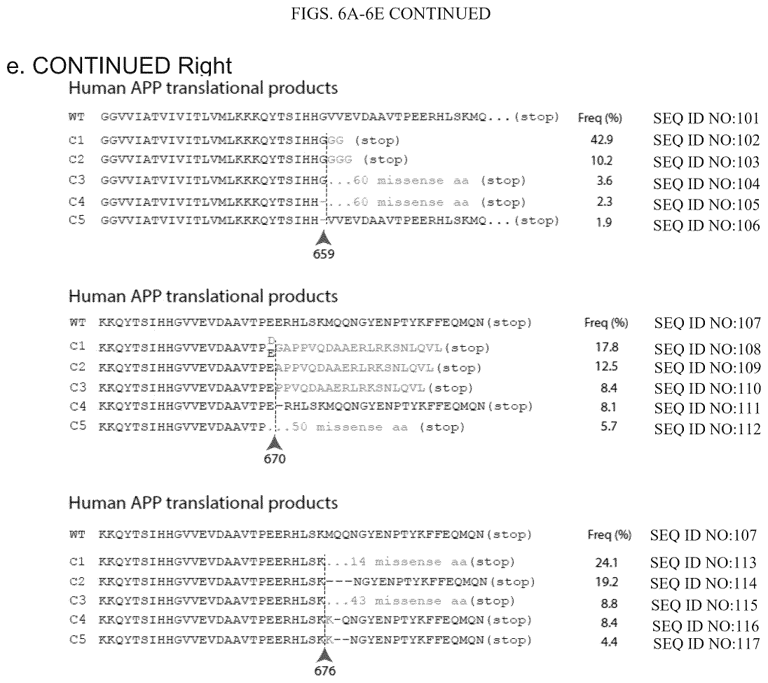

CRISPR Cas9 editing of APP C-terminus—The CRISPR/Cas9 system consists of a Cas9 nuclease enzyme that generates double-stranded breaks in DNA, and a custom-designed single guide-RNA (sgRNA) that targets the Cas9 to specific sites in the host genomic DNA. Typically, the synthetic sgRNAs are complementary to stretches of genomic DNA containing 3-nt PAM (protospacer adjacent motif) and flanking 20-nt sequences. Since subsequent repair after DNA-breaks is naturally error-prone, insertions and deletions (indels) are generated at the cut-sites, leading to disruption of the translational reading frame and effectively truncated proteins (reviewed in 14). We identified three PAM sites at the APP C-terminus that are conserved in both human and mouse, and synthesized sgRNAs targeting these regions (FIG. 1A). To compare the editing efficiency of these sgRNAs, we engineered a stable H4 neuroglioma cell line expressing single copies of APP:VN and BACE-1:VC (APP/BACE^{single_copy}), where editing efficiency of a given sgRNA could be determined as a simple fluorescence on/off readout and the effect of APP truncation could be assessed by evaluating secreted Aβ (for details, see FIGS. 6A and 6B and methods). The APP-sgRNA predicted to cut human APP at the 659 aa. (amino acid) position was the most efficient—both in editing APP as well as in attenuating Aβ—and also led to minimal indels (FIGS. 6C-6E). Accordingly, we used the APP659-sgRNA for further characterization (henceforth called ‘mo-APP-sgRNA’ or ‘hu-APP-sgRNA’ representing mouse and human specific sequences).

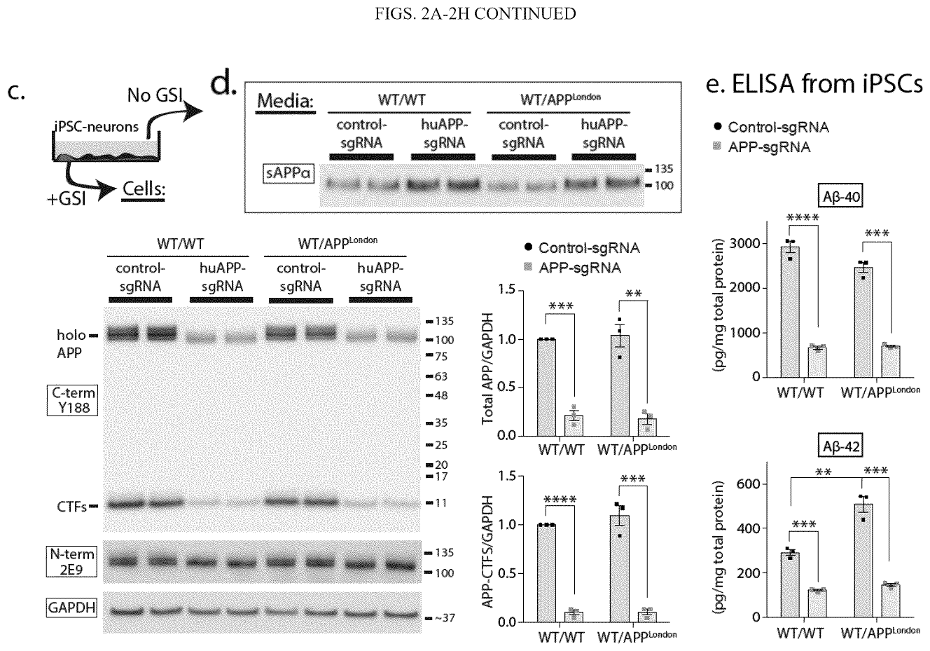

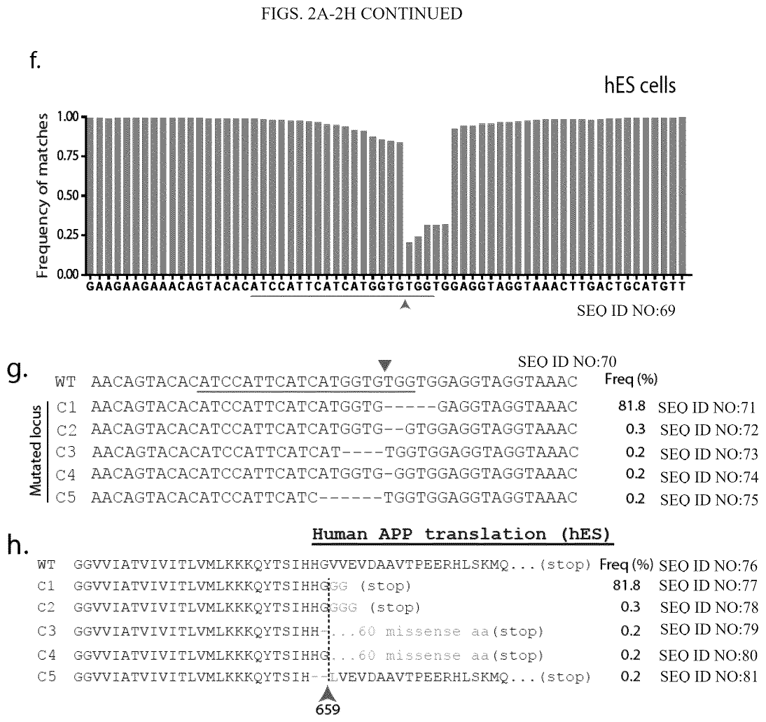

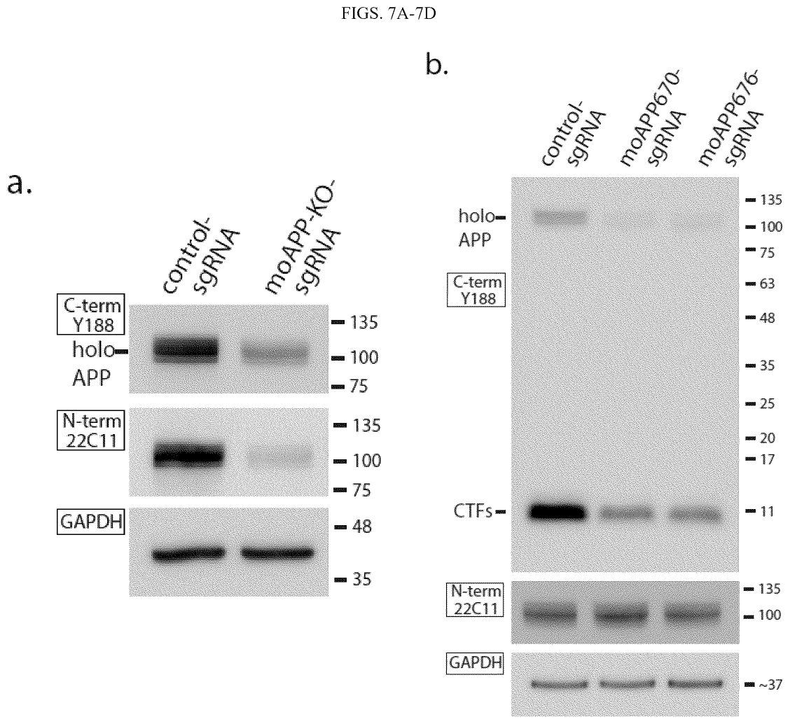

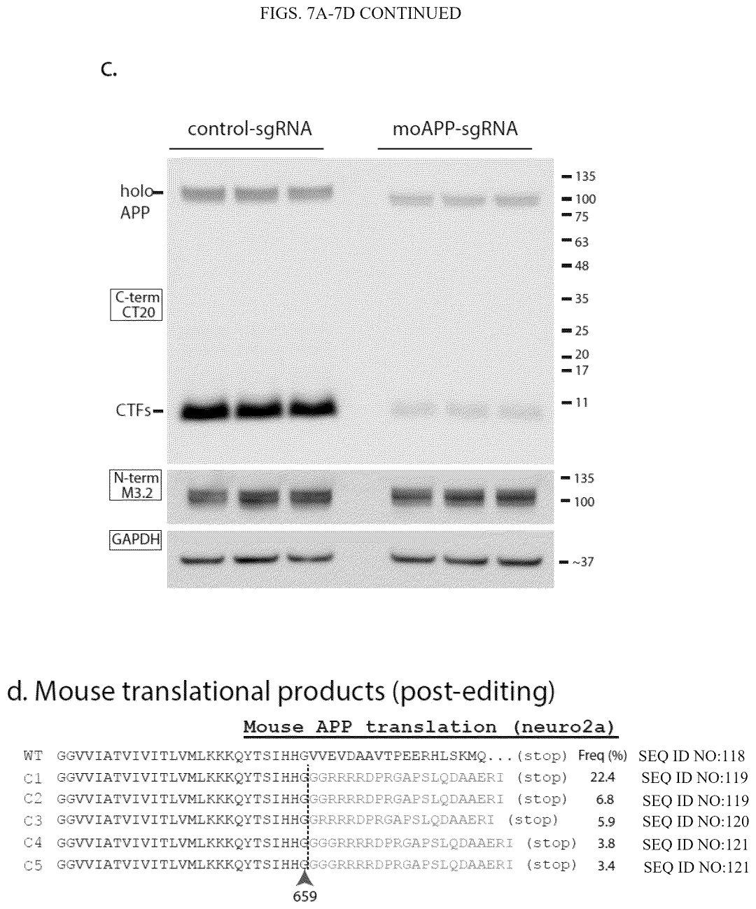

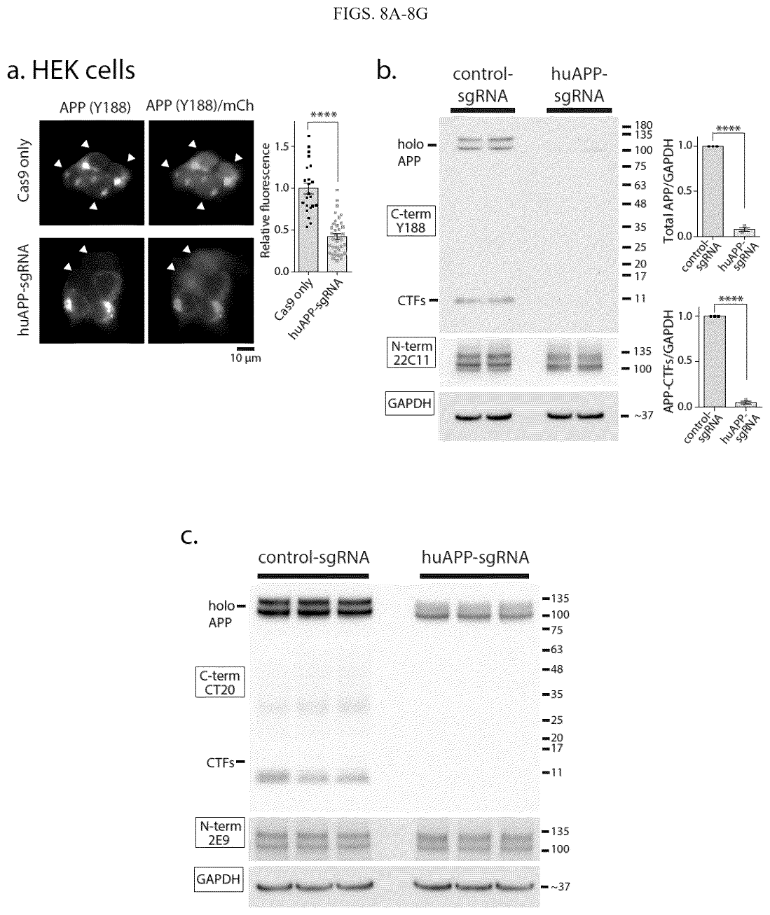

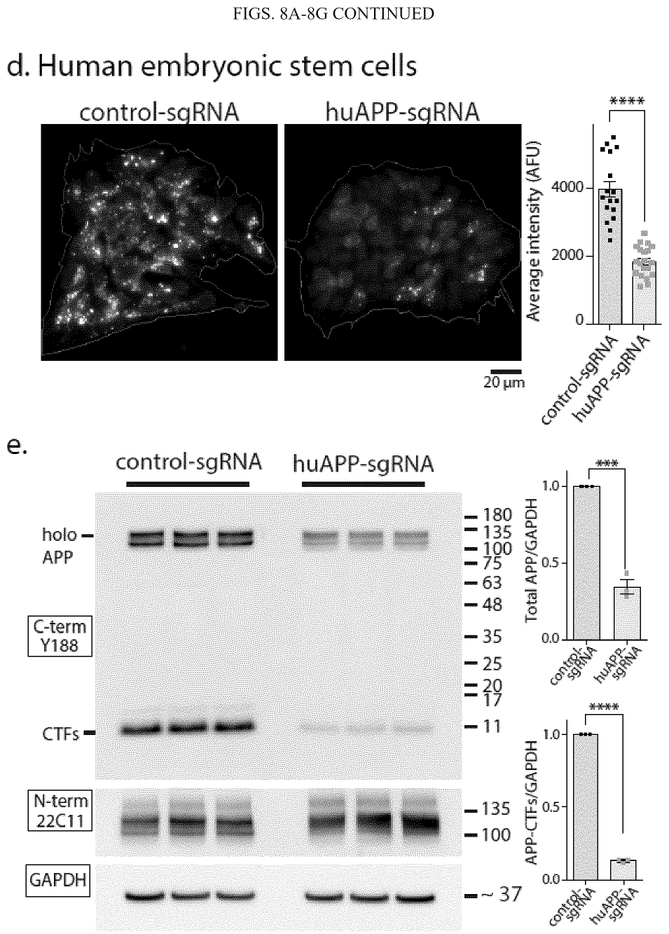

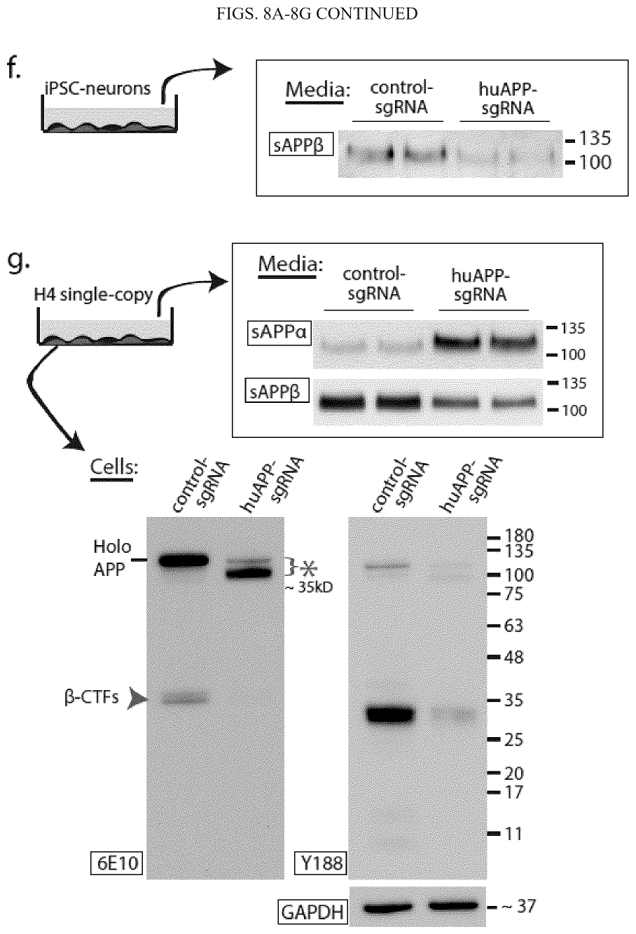

The TGG PAM and preceding 20-nt genomic target sequence recognized by the mo-APP-sgRNA is shown in FIG. 1A (top right); and FIGS. 1B-1F shows gene editing by this sgRNA in mouse cells. Note that upon editing, the Y188 antibody—recognizing the last 20 amino acids. of APP—would not be able to identify the resultant translational product. Robust editing of endogenous APP was seen in mouse neuroblastoma cells, as determined by attenuation of immunofluorescence with the Y188 antibody (FIG. 1), and decreased Y188-signal in western blots (FIGS. 1C-1D; FIG. 1E shows time-course of editing). Note that the edited APP is recognized by antibodies to the N-terminus, indicating selective editing of the C-terminus by the APP-sgRNA (FIGS. 1C and 1E). However, the N-terminus antibody was unable to detect APP when the entire gene was deleted (FIG. 7A). Similar results were obtained with other sgRNAs targeting APP C-terminus and other C- and N-terminus APP antibodies (FIGS. 7B and 7C). Genomic deep-sequencing confirmed efficient editing of mouse APP at the expected loci, APP-659 (FIG. 1F). Post-editing translational products show that the last 36 amino acids. are effectively truncated by APP-sgRNA (FIG. 7D). Though the TGG PAM at this site is conserved in both mouse and human APP, and the upstream sgRNA-targeting sequences only differ by two nucleotides (FIG. 2A, arrowheads); the mouse APP-sgRNA was unable to edit human APP (not shown). However, a sgRNA specific to the human APP targeting sequence robustly edited APP in HEK293 (FIGS. 8A-8C), as well as in human embryonic stem cells (FIGS. 8D-8E). CRISPR editing of APP did not alter the steady-state levels of holo-APP (note data throughout with multiple N-terminus antibodies in various cell lines).

Reciprocal manipulation of the APP β/α pathway by CRISPR Cas9 editing—Next, we examined APP editing in human iPSC-derived neurons. As shown in FIG. 2B, immunostaining with the Y188 antibody was attenuated in iPSC-neurons transduced by the hu-APP-sgRNA. To examine effects of APP editing in an “AD-like setting”, we also tested the hu-APP-sgRNA in a heterozygous knock-in iPSC line carrying the most common familial AD mutation (APPV717I, also called the ‘London mutation’; see methods for details of iPSC line). Both cell-lysates and supernatants were examined, to look for cellular and secreted APP products (see schematic in FIG. 2C). Immunoblotting with the Y188 antibody confirmed robust—and C-terminus selective—APP editing in both WT and APP-London iPSC lines (FIG. 2C). Examination of supernatants revealed that interestingly, APP-editing also led to increased sAPPα in both WT and London lines (FIG. 2D); suggesting upregulation of the neuroprotective a-cleavage pathway. ELISAs and western blot showed attenuated secretion of Aβ40/42 (FIG. 2E) and sAPPβ (FIG. 8F), confirming inhibition of the amyloidogenic pathway in these neurons. Genomic deep sequencing showed efficient editing of human APP by the sgRNA, with truncation of the last 36 amino acids. in human embryonic stem cells (FIGS. 2F-2H).

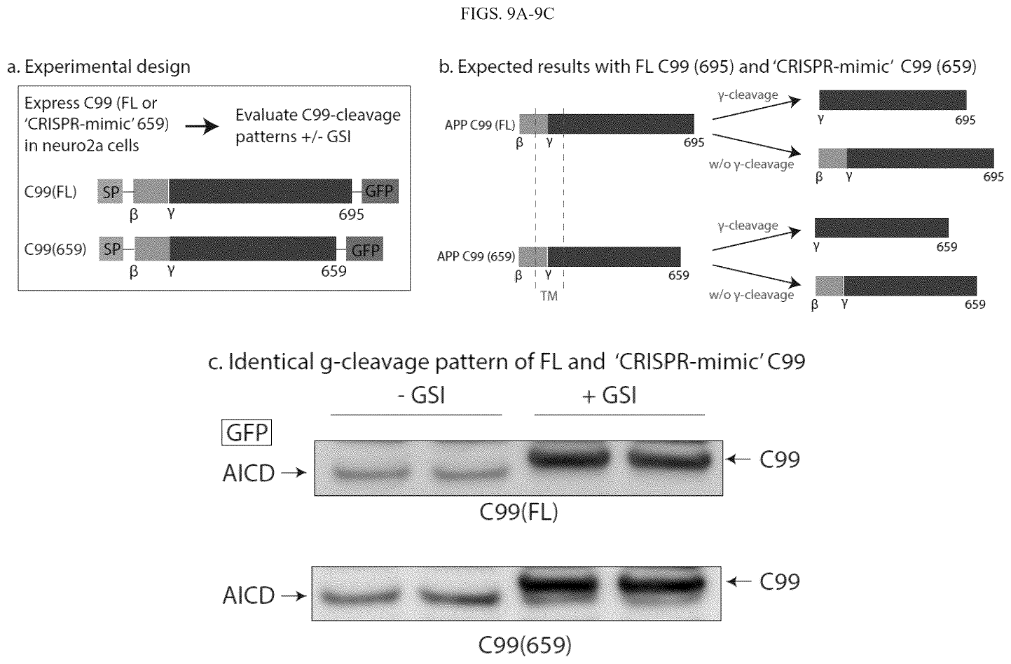

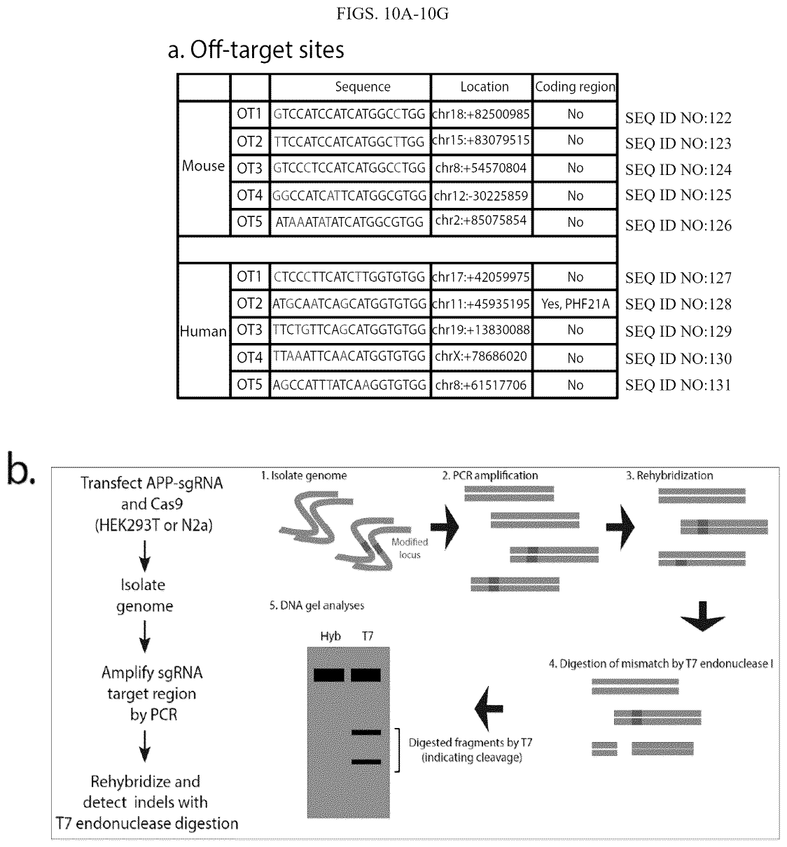

The data from iPSC-neurons suggest that the APP-sgRNA has reciprocal effects on APP β- and α-cleavage. To validate this in a more controlled setting, we tested the effects of APP editing in the H4 APP/BACE^{single_copy} cell line, where APP-cleavage is tightly regulated. In line with the data from iPSC-neurons, the hu-APP-sgRNA had reciprocal effects on APP β- and α-cleavage in APP/BACE^{single_copy} cells as well, confirming that our editing strategy has reciprocal effects on β/α cleavage (FIG. 8G). Further experiments using an APP-C99 construct (wild-type and truncated construct mimicking the CRISPR-product, APP-659) precludes an effect of the sgRNA on APP-γ-cleavage (FIGS. 9A-9C), indicating that our editing strategy is selectively affecting APP β-cleavage. Collectively, the available data strongly suggest that our gene editing strategy targeting the APP C-terminus is favorably manipulating the amyloid pathway by attenuating APP β-cleavage, while reciprocally up-regulating protective α-cleavage.

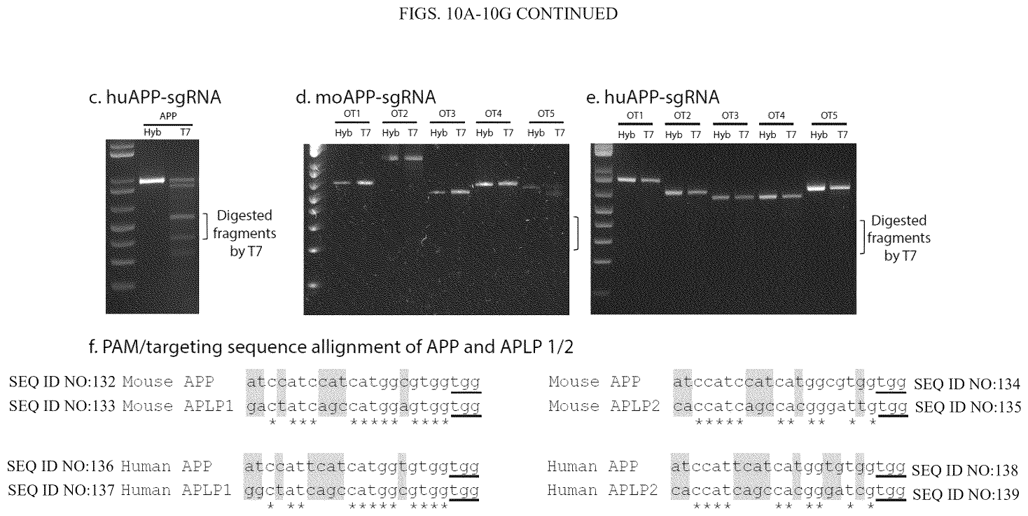

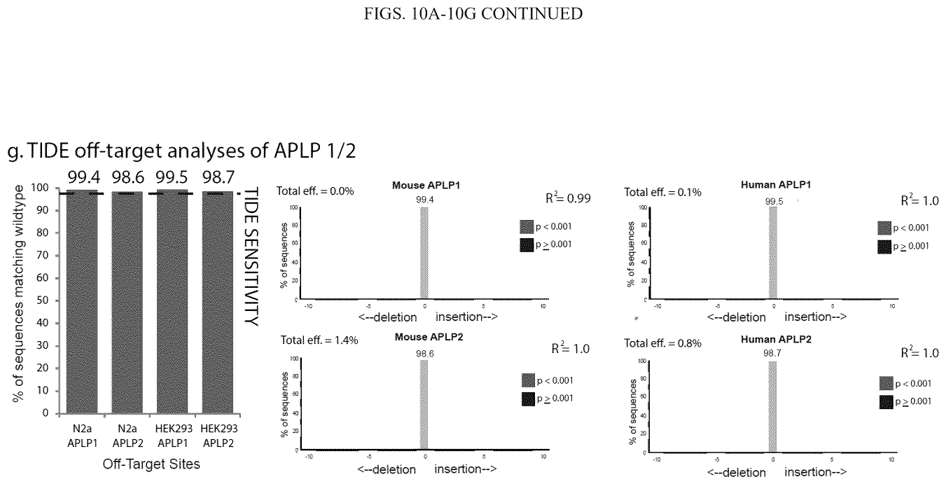

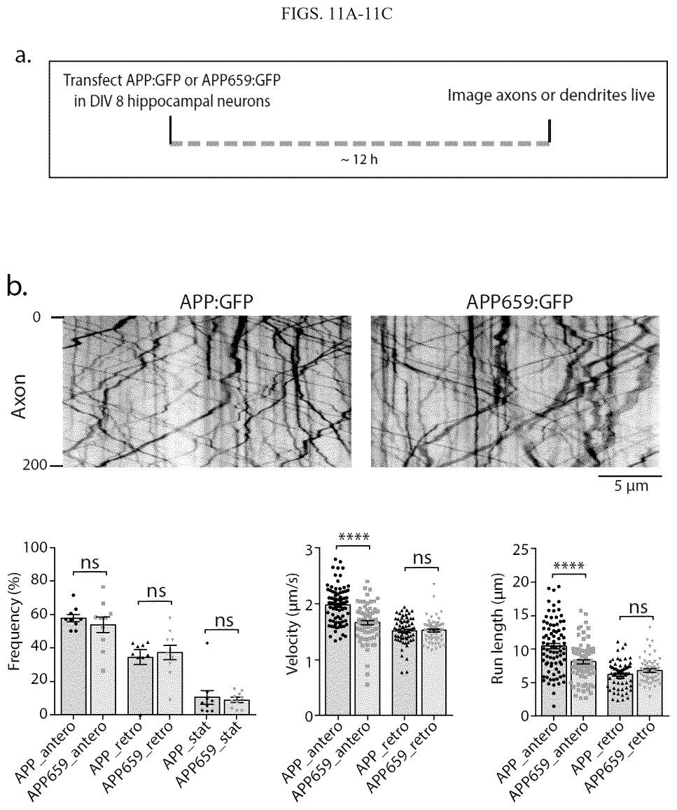

Off-target analysis and effect of APP C-terminus editing on neuronal physiology—Off-target effects of CRISPR/Cas9, due to unwanted editing of DNA-stretches resembling the targeted region, are a concern. Towards this, we asked if our mouse and human APP-sgRNA were able to edit the top five computationally predicted off-target sites (FIG. 10A; also see Table 3). No editing was seen using T7 endonuclease assays (FIGS. 10B-10E). Though APP null mice are viable, there is compensation by the two APP homologues APLP1 and 2 that undergo similar processing as APP (15,16). APLP1 and 2 were not amongst the top 50 predicted off-target sites, as their corresponding sgRNA-target sites were substantially different from APP (see sequences in FIG. 10F). For further assurance that our sgRNA was not editing the APP homologues, we performed specific off-target TIDE (Tracking of Indels by DEcomposition) analyses (17) on cells carrying the sgRNA. As shown in FIG. 10G, TIDE analyses showed no editing of APLP 1/2 by the sgRNA.

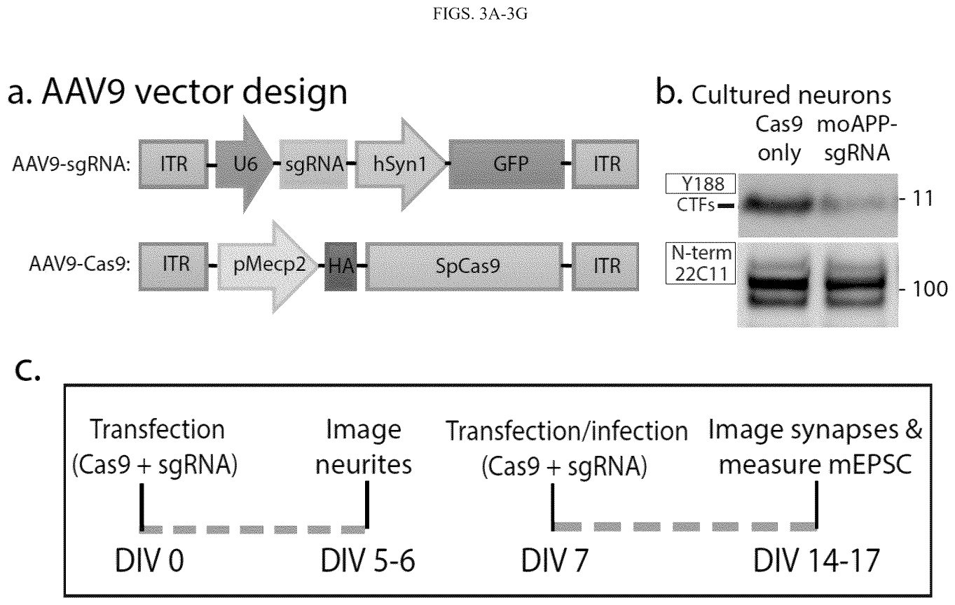

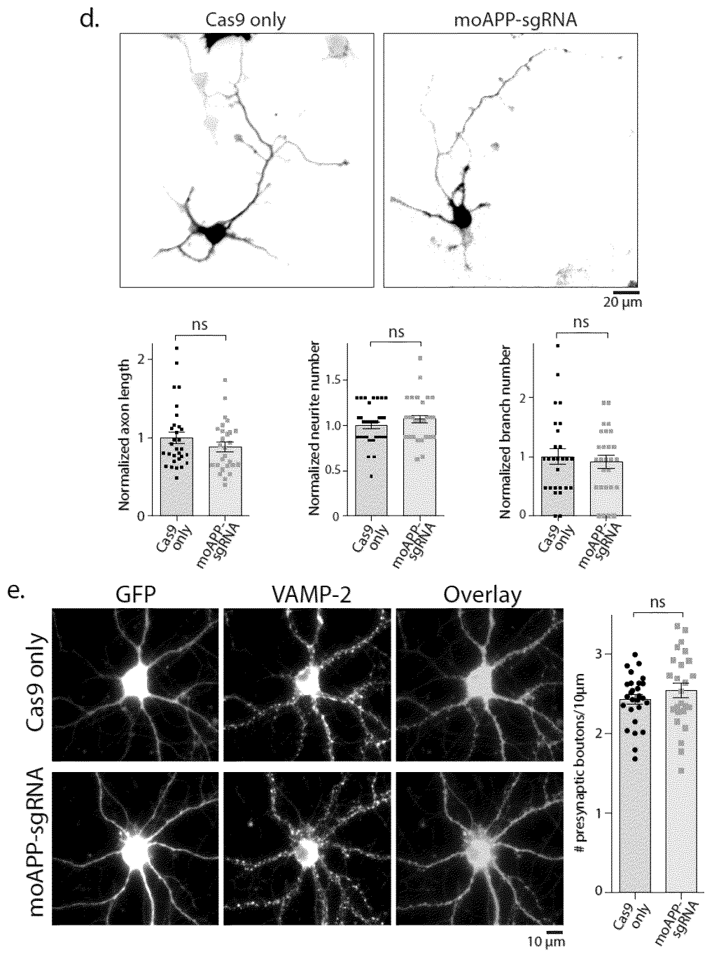

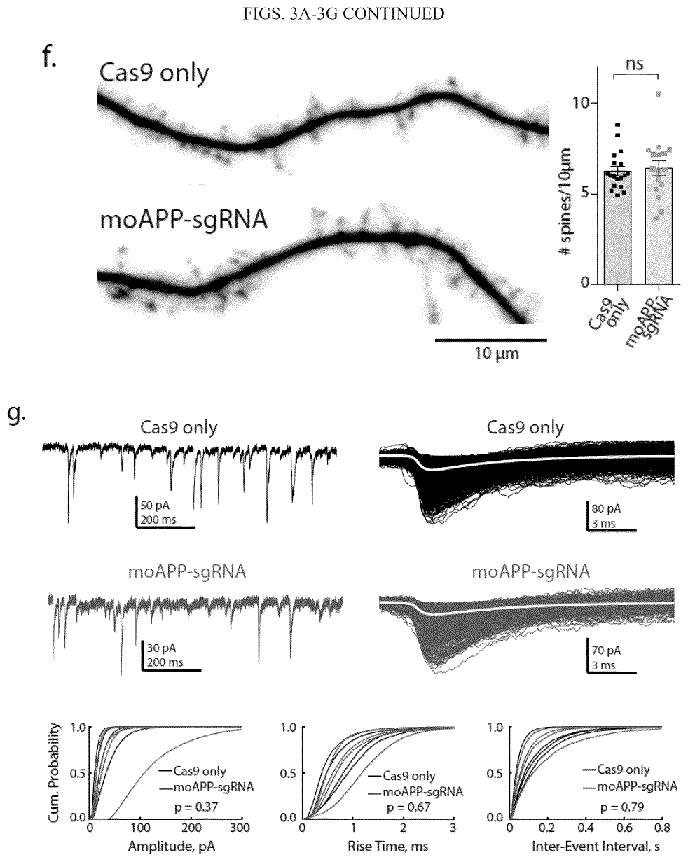

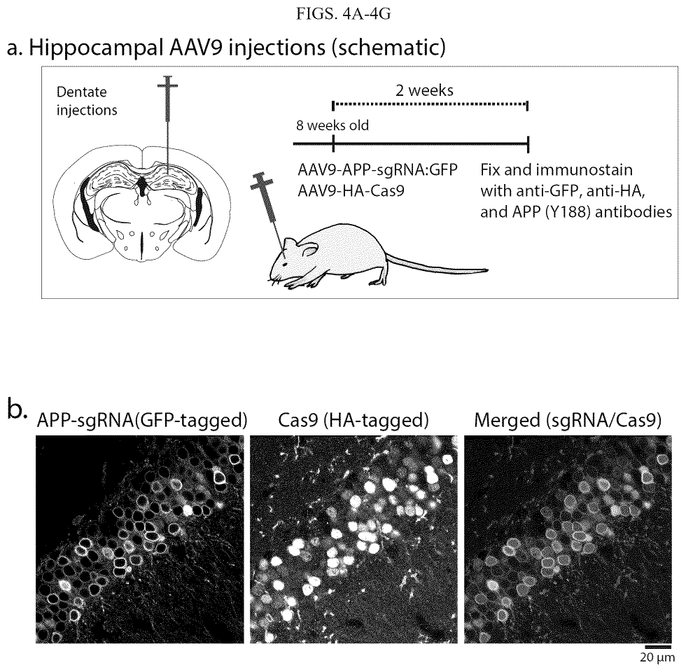

APP has known physiologic roles in axon growth and signaling (18). As noted above, the N-terminus of APP—thought to play roles in axon growth and differentiation—is entirely preserved in our setting. The C-terminal APP intracellular domain (AICD) has been implicated in gene transcription, though the effect appears to be both physiologic and pathologic (19,20.) To examine potential deleterious effects of editing the extreme C-terminus of APP, we turned to cultured hippocampal neurons where various parameters like neurite outgrowth and synaptic structure/function can be confidently evaluated. To study pre-synapse structure and neuronal activity, we generated AAV9 viruses carrying the mo-APP-sgRNA and Cas9, tagged with GFP and HA respectively (see vector design in FIG. 3A) that transduced almost all cultured neurons (FIG. 3B and data not shown). In blinded analyses, we found no significant effect of the mo-APP-sgRNA on neurite outgrowth, axon-length, synaptic organization, or neuronal activity (FIGS. 3C-3G). We reason that the lack of deleterious effects upon editing is likely because: 1) most of the APP molecule remains intact after editing; 2) the APP homologues APLP1/2—that undergo similar processing as APP, generate CTFs, and are known to compensate for APP function—remain unedited; and 3) APP-cleavage is not entirely blocked by our approach.

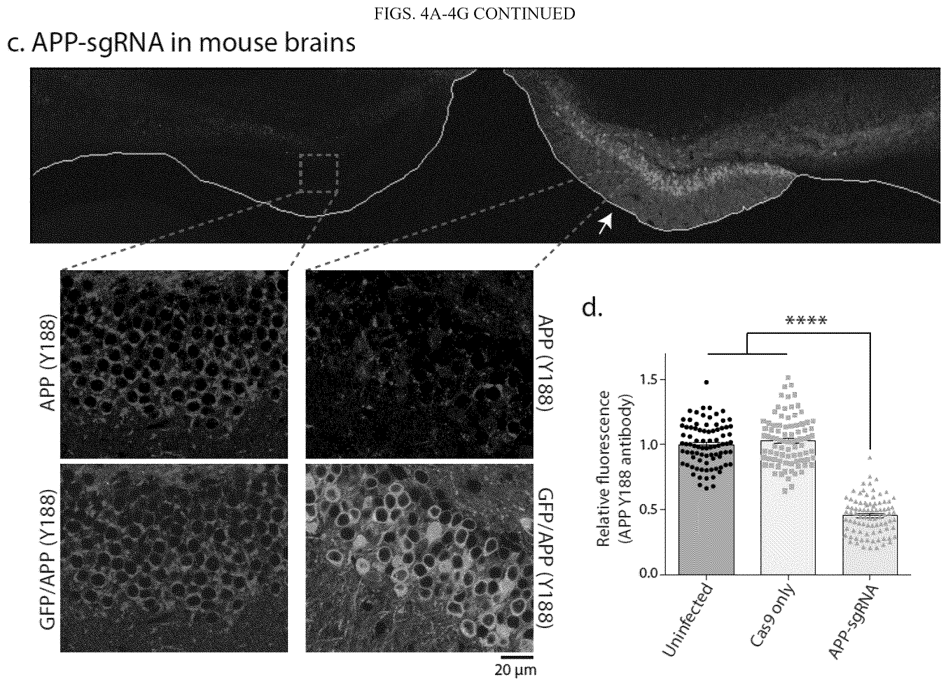

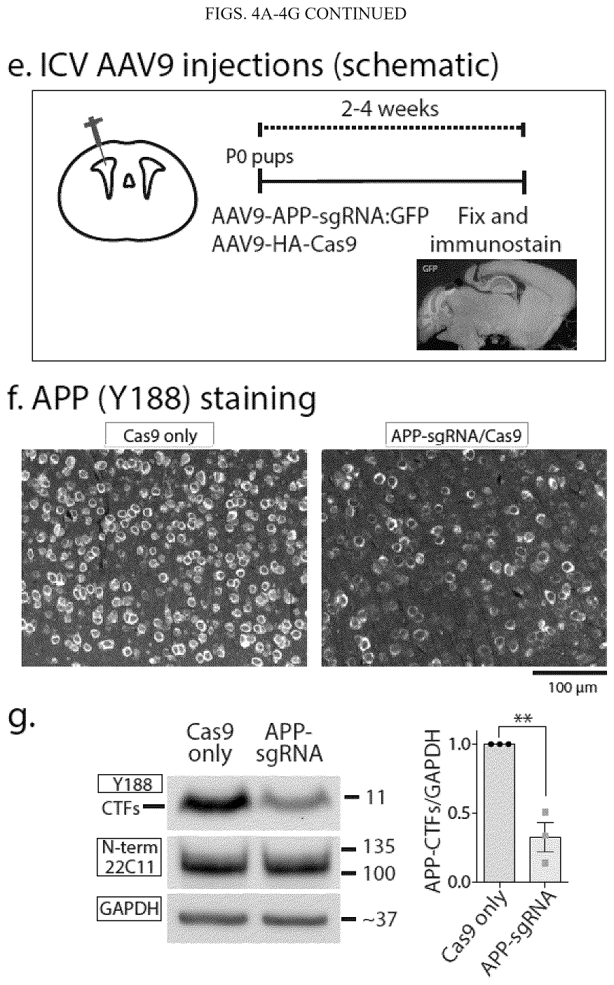

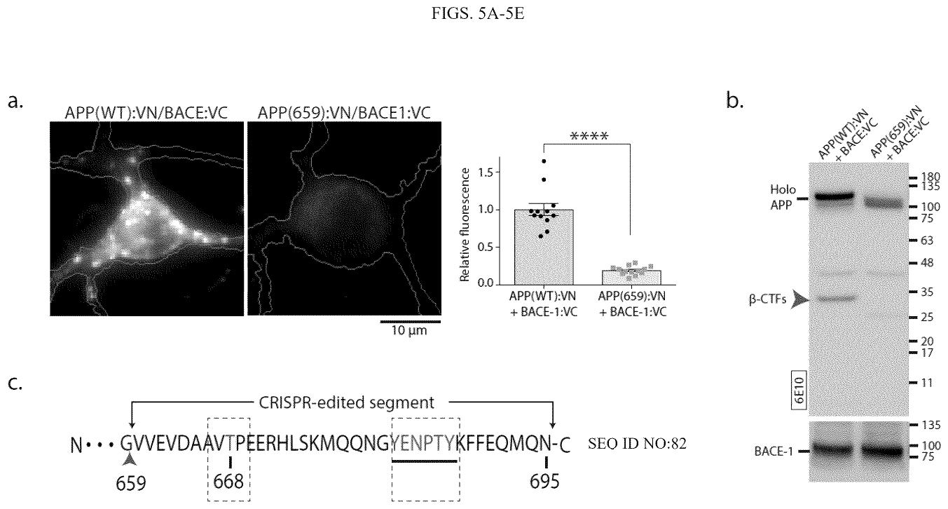

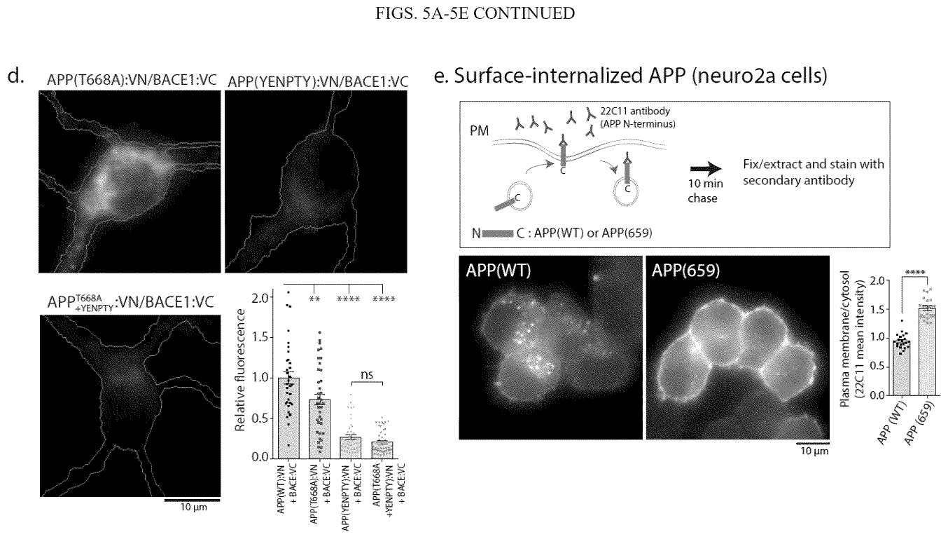

Editing of APP C-terminus in vivo and mechanistic details of APP β/α manipulation—Next we asked if the APP-sgRNA could edit endogenous APP in mouse brains. Injection of the AAV9s into mouse hippocampi (FIG. 4A) led to efficient transduction of both sgRNA and Cas9 in dentate neurons (86.87±2.83% neurons carrying the sgRNA also had Cas9; see representative images in FIG. 4B). Immunostaining of transduced neurons with the APP Y188 antibody showed attenuated staining, suggesting editing of endogenous APP in vivo (FIGS. 4C and 4D). To achieve a more widespread expression of the sgRNA and Cas9 in mouse brains—and also evaluate editing by biochemistry—we injected the viruses into the ventricles of neonatal (P0) mice and examined the brain after 2-4 weeks (FIG. 4E). Previous studies have shown that when AAVs are injected into the ventricles of neonatal mice, there is widespread delivery of transgenes into the brain—also called somatic transgenesis (21,22). Indeed, APP Y188 immunostaining was attenuated in cortical regions (FIG. 4F) and immunoblotting with the Y188 antibody also showed a decreased signal (FIG. 4G); indicating that the APP-sgRNA can edit APP in vivo.

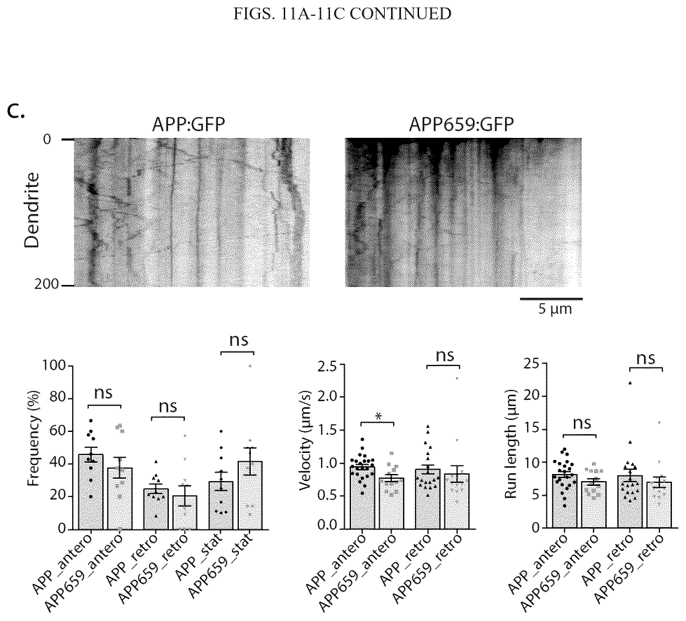

To determine the mechanism by which the APP-sgRNA manipulates the amyloid pathway, we used a “CRISPR-mimic” truncated APP construct (APP-659) that is the major post-editing translational product in both mouse and human cells (see FIG. 2H, FIG. 6E, and FIG. 7D). Using our BifC assay (10), we first asked if the CRISPR-mimic APP-659 interacted with BACE-1. APP-659/BACE-1 approximation was greatly attenuated in cultured neurons (FIG. 5A), along with a decrease in R-CTF generation (FIG. 5B). Next, we visualized axonal and dendritic transport of APP-WT and APP-659. Although there were minor changes (FIGS. 11A-11C and Table 4), it seems unlikely that such small transport perturbations would lead to the dramatic attenuation of β-cleavage and Aβ-production seen in our experiments.

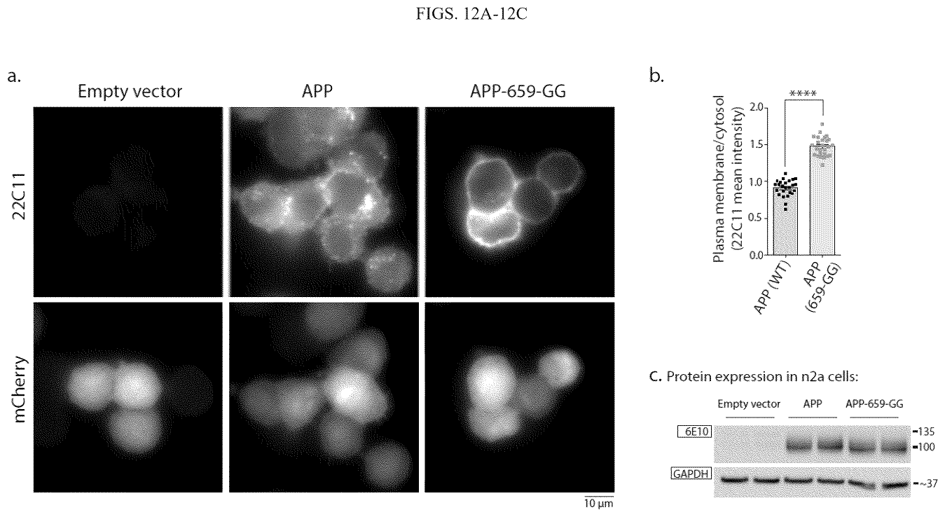

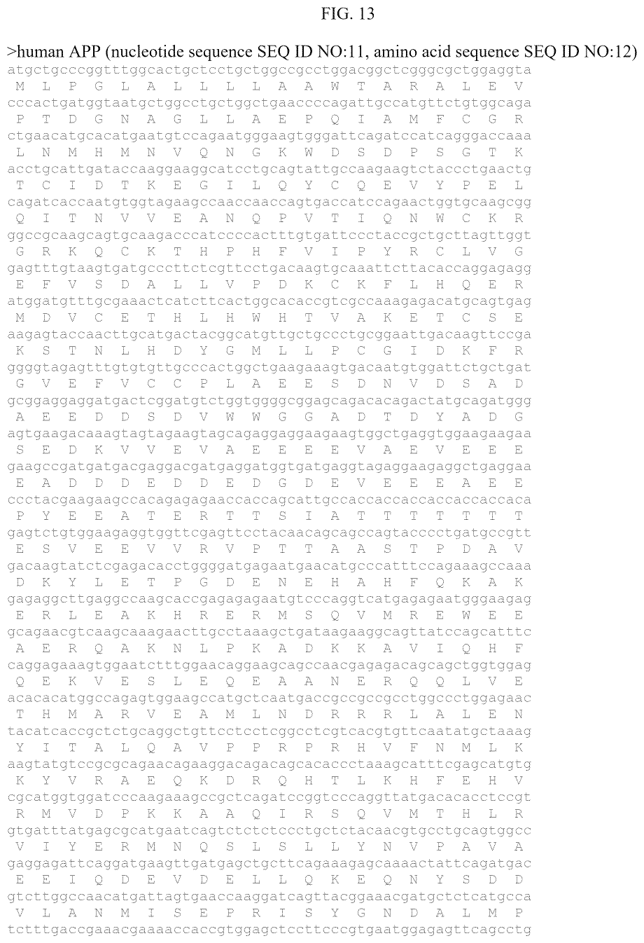

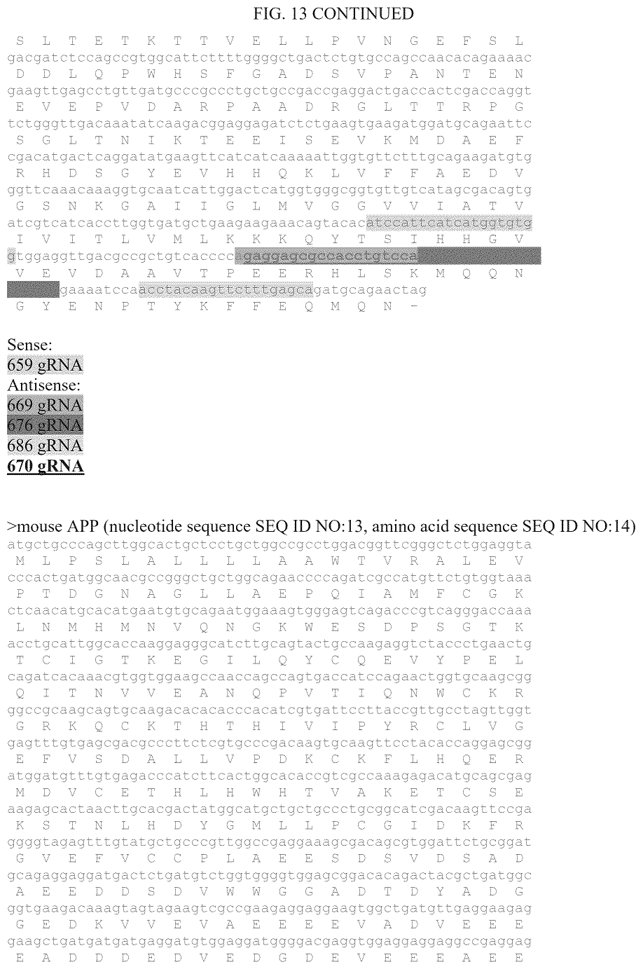

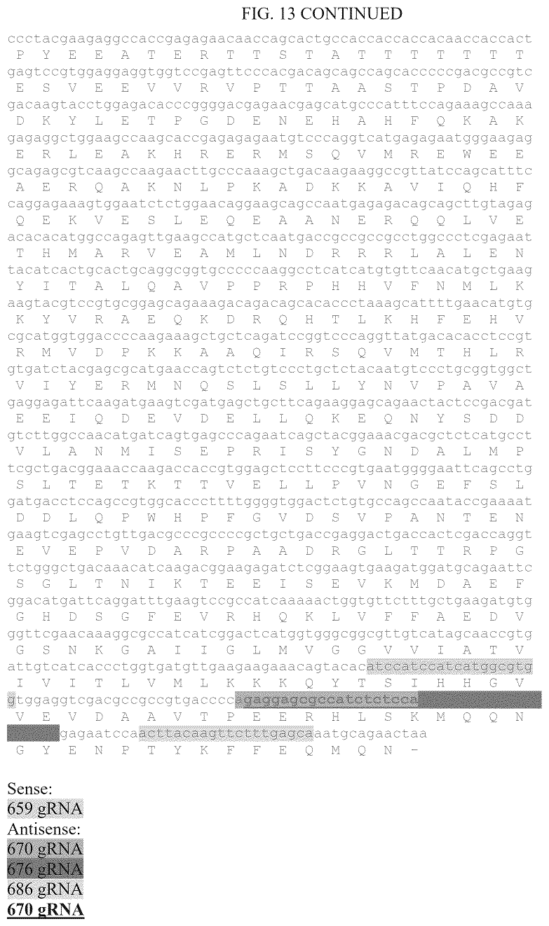

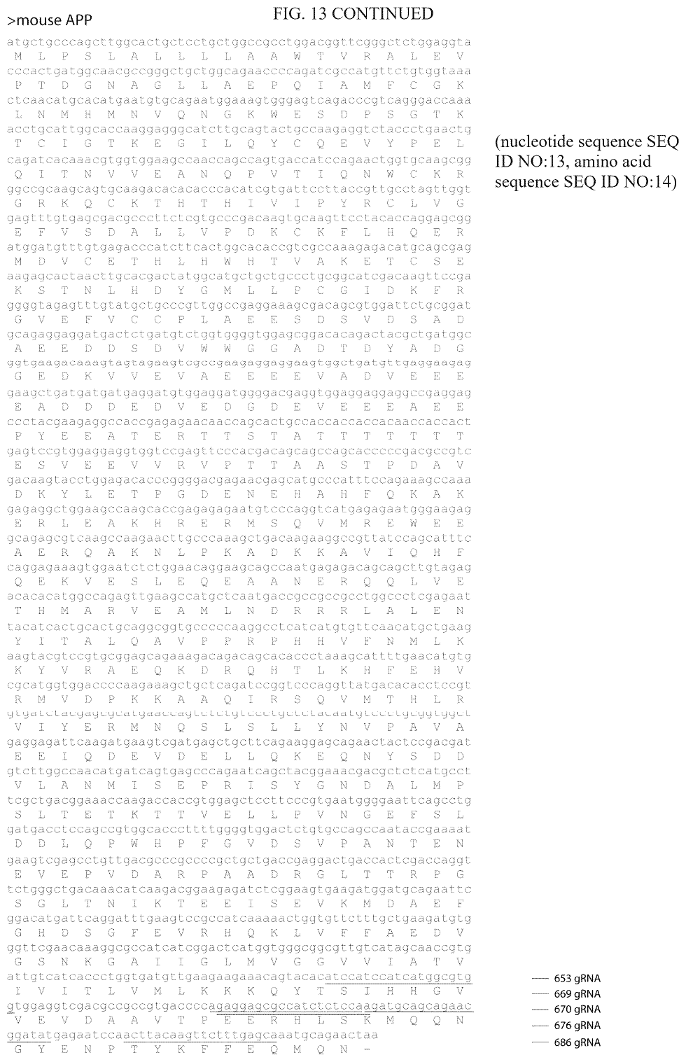

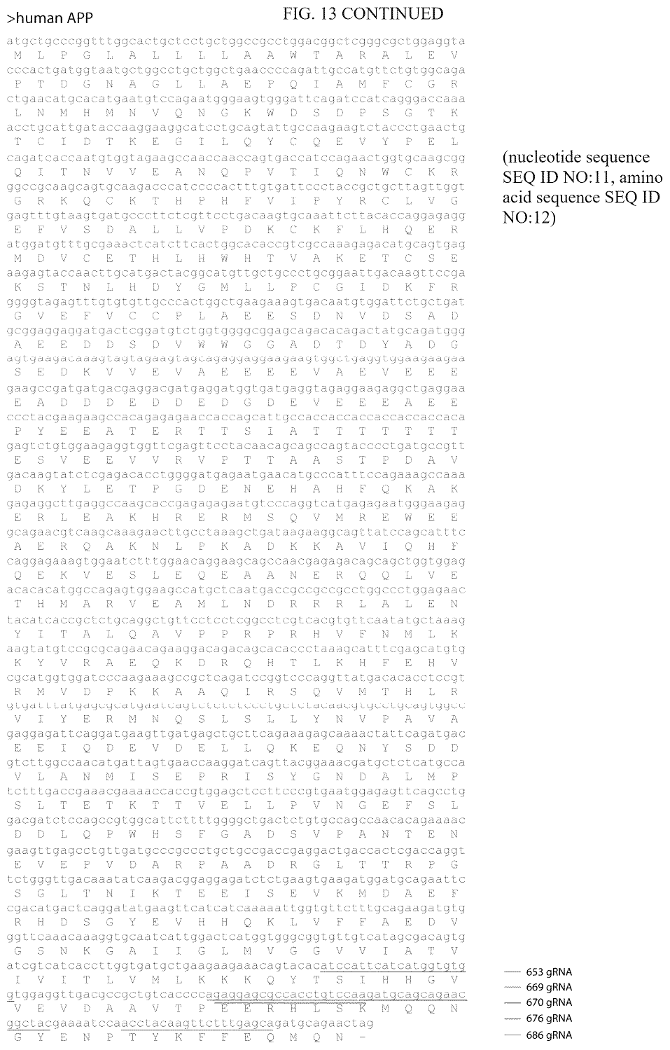

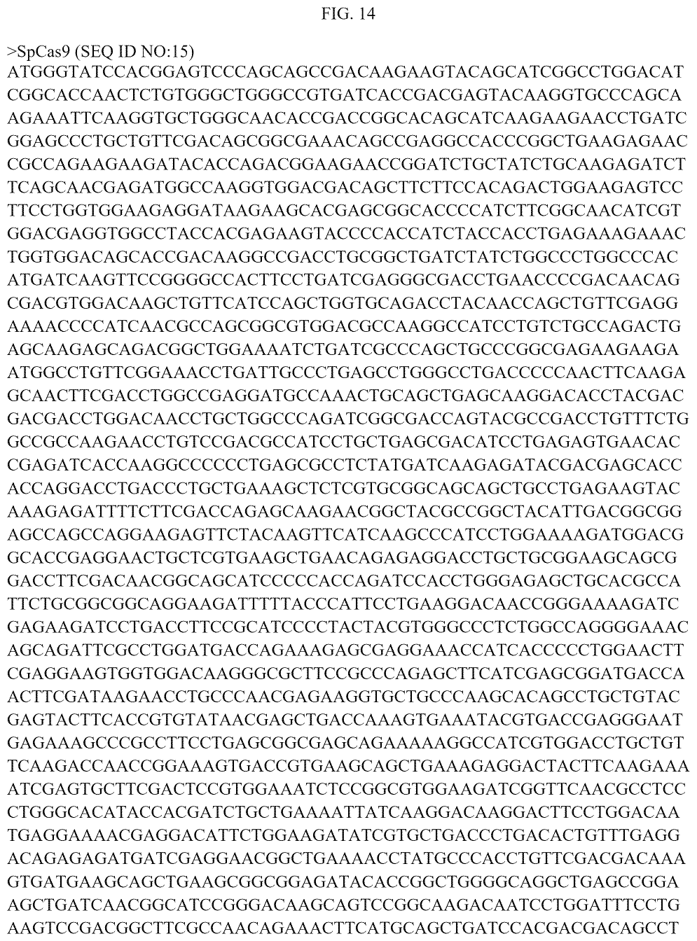

The CRISPR-edited segment of APP contains the residues T668 and Y682-Y687 (YENPTY motif, see FIG. 5C; also present in APLP1/2), that have been reported to play a role in Aβ production (12,23,24). Specifically, APP phosphorylated at T668 has been reported to colocalize with BACE-1 in endosomes (23), and the YENPTY motif is known to mediate APP internalization from the plasma membrane (25). Examining the effects of these residues in APP/BACE-1 BifC assays, we saw that the extent of APP/BACE-1 attenuation by the YENPTY mutation strongly resembled the decrease in fluorescence complementation by the APP-659 construct (FIG.>5D). A prediction from these experiments is that endocytosis of the CRISPR-mimic APP from the cell surface should be attenuated; and indeed, this was the case in internalization assays (FIG. 5E). Similar results were seen with an “APP-659-GG” construct that more closely resembles the most common post-editing translational product of our sgRNA (FIGS. 12A-12C; also see post-editing products from human cells in FIG. 2H and FIG. 6E).

Collectively, the data suggest that our gene-editing approach does not have a major effect on post-Golgi trafficking of APP, but attenuates its endocytosis from cell surface, and consequently, its interaction with BACE-1 in endosomes—though we cannot exclude a direct effect of editing on APP/BACE-1 interaction. This is also consistent with previous studies showing that surface APP is internalized into endosomes, where it is cleaved by BACE-1 (26-29). Since most of the APP α-cleavage is thought to occur at the cell surface (30), this may also explain why the non-amyloidogenic pathway is enhanced by our approach.

Using CRISPR/Cas9 technology, herein we provide conceptual proof for a strategy that selectively edits the C-terminus of APP and alters the balance of APP-cleavage—attenuating β-cleavage and Aβ, while upregulating neuroprotective α-cleavage. The N-terminus of APP—known to play physiologic roles—is unaffected, along with the compensatory APP homologues APLP1/2. No deleterious effects were seen in neurophysiologic parameters. Without wishing to be bound by any particular theory, our strategy likely works by editing the terminal YENPTY motif in APP that is responsible for its internalization, subsequent APP/BACE-1 association, and initiation of the amyloidogenic pathway; while retention of APP at the plasma membrane may facilitate the upregulation of APP α-cleavage.

APP processing is regulated by α-, β—, and γ-secretases; and the various cleavage products may play physiological functions that are not fully understood (31,32). Previous studies suggest that in vivo deletion of the APP C-terminus blocks APP β-cleavage without obvious effects on neuroanatomy, behavior and neuronal activity in adult mice (13). Notably, the APP homologues APLP 1/2 also have YENPTY motifs (15,16)—that can presumably undergo endocytosis and protein-protein interactions—and are expected to compensate for the loss of the C-terminus. The precise reasoning behind enhanced α-cleavage is unclear. We propose that retention of APP at the plasma membrane might be responsible, but we cannot rule out other causes, including off-target effects, and further detailed studies may provide clarity.

### METHODS

Constructs, antibodies and reagents—For transient co-expression of CRISPR/Cas9 components, APP sgRNA nucleotides were synthesized and cloned into pU6-(Bbs1)_CBh-Cas9-T2A-mCherry vector at Bbs1 site. For viral transduction, a dual vector system was used to deliver CRISPR/Cas9 components using AAV9 (33). For making the AAV9 vectors, the APP sgRNA was cloned into pAAV9-U6sgRNA(SapI)_hSyn-GFP-KASH-bGH vector at Sap1 site. The CRISPR/Cas9 stable cell lines were generated by lentivirus infection as follows. The APP sgRNA was cloned into lentiCRISPR v2 vector at Bbs1 site to produce lentivirus (34). For making APP deletions and relevant constructs, the human APP659 truncation was PCR amplified and cloned at Hind3 and Sac2 sites of pVN to generate pAPP659:VN. The BBS-APP659 was PCR amplified and cloned into pBBS-APP:GFP at Hind3 and Sac2, replacing BBS-APP, to generate pBBS-APP659:GFP. The pBBS-APP^{YENPTY}:GFP was generated by site directed mutagenesis from pBBS-APP:GFP. The pAPP^{T668A}:VN and pAPP^{T668A+YENPTY}:VN were generated by site directed mutagenesis from pAPP:VN and pAPP^{YENPTY}:VN. Antibodies used were as follows: APP Y188 (ab32136; Abcam), APP 22C11 (MAB348; Millipore), APP 6E10 (803001; BioLegend), APP M3.2 (805701; BioLegend), APP 2E9 (MABN2295; Millipore), APP CT20 (171610; Millipore), sAPPβ (18957; IBL) BACE-1 (MAB931; R&D), GAPDH (MA5-15738, ThermoFisher), GFP (ab290, Abcam), GFP (A10262, Invitrogen), HA (901513, BioLegend), VAMP2 (104211, Synaptic Systems). Reagents were as follows: γ-secretase inhibitor BMS-299897 (Sigma), and Rho Kinase (ROCK)-inhibitor H-1152P (Calbiochem).

### Cell Cultures, Transfections, Viral Production Infections, and Biochemistry—

HEK293 and neuro2a cells (ATCC) were maintained in DMEM with 10% FBS. Cells were transfected with Lipofectamine™ 2000 and collected 5 days after transfection for biochemical and immunostaining analysis. All the studies involving primary neuron culture were performed in accordance with University of Wisconsin guidelines. Primary hippocampal neurons were obtained from postnatal (P0-P1) CD1 mice (either sex), and transiently transfected using Lipofectamine™ 2000 or Amaxa™ 4D system (Lonza). Dissociated neurons were plated at a density of 30,000 cells/cm² on poly-D-lysine-coated glass-bottom culture dishes (Mattek) and maintained in Neurobasal™/B27 medium with 5% CO₂. For APP/BACE-1 interaction and APP transport studies, DIV 7 neurons were cultured for ˜18-20 h after transfection. For spine density analysis, DIV7 neurons were transfected with Cas9, sgRNA and soluble marker, and cultured for 7 d before imaging. For testing the effect of CRISPR/Cas9 on neuronal development, neurons were electroporated with the respective constructs before plating using an Amaxa™ 4D-Nucleofector™ system with the P3 Primary Cell 4D-Nucleofector™ X kit S and program CL-133.

For western blotting, pre-synapse analyses and electrophysiology, DIV7 cultured neurons were infected with either AAV9-APP sgRNA-GFP (2.24×10¹³ Vg/ml) and AAV9-Cas9 (2.4×10¹⁴ Vg/ml), or AAV9-GFP (2.58×10¹³ Vg/ml) and AAV9-Cas9 at a multiplicity of infection (MOI) of 1.5×10⁵. Neurons were analyzed 7 days post-infection. Lentivirus was produced from HEK293FT cells as described (35). Briefly, HEK293FT cells (Life Technologies) were maintained in DMEM with 10% FBS, 0.1 mM NEAA, 1 mM sodium pyruvate and 2 mM Glutamine. Cells were transfected with lentiviral-target and helper plasmids at 80-90% confluency. 2 days after transfection, the supernatant was collected and filtered with 0.45 μm filter. For experiments with hESCs, cells were cultured on a Matrigel® substrate (BD Biosciences) and fed daily with TeSR-E8 culture media (StemCell Technologies). When the cells were around 60-70% confluent, they were infected with a 50/50 mixture of TeSR-E8 (with 1.0 μM H-1152P) and lentivirus supernatant. After 24 h, the virus was removed, and the cells were fed for 2 days (to recover). After 3 days, cells were treated with 0.33 μg/mL of puromycin for 72 h to select for virally-integrated hESCs. For HEK and neuro2a cell lines, cells were infected with the lentivirus carrying APP-sgRNA and Cas9 for 24 h. And then cells were fed for 1 day to recover. After 2 days, cells were treated with 1 μg/mL of puromycin for 72 h to select for virally-integrated cells.

Human NPCs were generated as has been described previously (36), using manual rosette selection and Matrigel® (Corning) to maintain them. Concentrated lentiviruses express control-sgRNA or APP-sgRNA were made as described previously (37), using Lenti-X™ concentrator (Clontech). The NPCs were transduced with either control-sgRNA or APP-sgRNA after Accutase® splitting and were submitted to puromycin selection the subsequent day. Polyclonal lines were expanded and treated with puromycin for 5 more days before banking. Neuronal differentiations were carried out by plating 165,000 cells/12 well-well in N2/B27 media (DMEM/F12 base) supplemented with BDNF (20 ng/mL; R&D) and laminin (1 ug/mL; Trevigen).

For biochemistry, cell lysates were prepared in PBS+0.15% Triton™ X-100 or RIPA supplemented with protease inhibitor cocktail, pH 7.4. After centrifuging at 12,000 g for 15 min at 4° C., supernatants were quantified and resolved by SDS-PAGE for western blot analysis. For sAPPα and sAPPβ detection, cell culture medium was collected and centrifuged at 2,000 g for 15 min at RT. The supernatants were resolved by SDS-PAGE for western blot analysis; band intensities were measured by ImageJ. Human Aβ40 and Aβ42 were detected using kits, according to the manufacturer's instructions (Thermo KHB3481 and KHB3544). Briefly, supernatants from H4^{single copy} cells or human iPSC derived neurons were collected and diluted (×5 for H4 and ×2 for iPSC-neuron). The diluted supernatants and the human Aβ40/42 detection antibodies were then added into well and incubated for 3 h at RT with shaking. After washing (×4), the anti-Rabbit IgG HRP solution was added and incubated for 30 min at RT. The stabilized Chromogen was added after washing (×4) and incubated for another 30 min at RT in the dark. After addition of stop solution, absorbance at 450 nm was read using a luminescence microplate reader.

Developing a single-copy, stable APP BACE-1 cell line—H4 tetOff F1pIn empty clone was maintained in OptiMEM® with 10% FBS, 200 μg/mL G418 and 300 μg/mL Zeocin. To generate an APP:VN/BACE-1:VC stable cell line carrying single copies of APP and BACE-1, the expressing plasmid and pOG44 plasmids were transfected with Lipofectamine™ 2000. 2 days after transfection, cells were selected with 200 μg/mL hygromycin B and 200 μg/mL G418 for 1 week. A monoclonal cell line with stable expression was selected. H4 stable cell lines were then infected with the lentivirus carrying APP-sgRNA and Cas9, as described above. After 24 h, the virus was removed, and cells were fed for 1 day to recover. After 2 days, cells were treated with 0.7 μg/mL of puromycin for 72 h to select for virally-integrated cells.

Generation of the APPLondon (V717I) knockin iPSC line—CRIPSR/Cas9 was used to knock in the APP V717I mutation (APPLon) into a commercially available control human iPSC line IMR90 (clone 4, WiCell). sgRNAs targeting Exon17 of APP were designed using the CRISPR design tool created by Feng Zhang's lab and subcloned into the MLM3636 vector (AddGene). Efficacy of multiple sgRNAs was first assessed in HEK293 cells (Geneart™ Genomic Cleavage Detection Kit, Life Technologies). The ssDNA HDR template was designed to include a silent CRISPR blocking mutation at the PAM site of most efficacious sgRNA in addition to the APPLon mutation. sgRNA, Cas9-2A-mCherry (generously provided by Hynek Wicterle), and ssDNA HDR template were electroporated (Lonza Nucleofector™) into feeder-free IMR90 iPSCs, followed by cell sorting on mCherry signal and plating at low density on MEFs (MTI-GlobalStem). Individual clones were manually picked into a 96 well format, subsequently split into duplicate plates, one of which were used to generate gDNA as had been done previously³⁸. For each clone, exon 17 of APP was amplified and initially screened by restriction digest for the presence of a de novo Bcl1 site introduce by the APPLon mutation. Sanger sequencing was used to confirm the mutation, and successful knockin clones were expanded and banked. Potential off-target effects of CRISPR/Cas9 cleavage were analyzed by Sanger sequencing of the top 5 predicted off-target genomic locations, which demonstrated a lack of indels for multiple clones. Clone 88 was picked for future studies.

Immunofluorescence, microscopy image analysis, APP trafficking and endocytosis assays—For immunostaining of endogenous APP or VAMP2, cells were fixed in 4% PFA/sucrose solution in PBS for 10 min at room temperature (RT), extracted in PBS containing 0.2% Triton™ X-100 for 10 min at RT, blocked for 2 h at RT in 1% bovine serum albumin and 5% FBS, and then incubated with rabbit anti-APP (1:200) or mouse anti-VAMP2 (1:1000) diluted in blocking buffer for 2 h at RT. After removal of primary antibody, cells were blocked for 30 min at RT, incubated with goat anti-rabbit (Alexa Fluor 488) or goat anti-mouse (Alexa Fluor® 594) secondary antibody at 1:1000 dilution for 1 h at RT and then mounted for imaging. z-stack images (0.339 μm z-step) were acquired using an inverted epifluorescence microscope (Eclipse Ti-E) equipped with CFI S Fluor VC 40× NA 1.30 (Nikon). An electron-multiplying charge-coupled device camera (QuantEM: 512SC; Photometrics) and LED illuminator (SPECTRA X; Lumencor) were used for all image acquisition. The system was controlled by Elements software (NIS Elements Advanced Research). z-stacks were subjected to a maximum intensity projection. For APP Y188 staining, the average intensity of single cell body (neuro2A, HEK293 and neurons) or the whole colony (hESCs) was quantified. All the images were analyzed in Metamorph® and ImageJ.

Spine density experiments were done as described previously (39). Briefly, DIV 7 neurons were transfected with desired constructs for 7 days, and secondary dendrites were selected for imaging. z-stack images were captured using a 100× objective (0.2 μm z-step) and subjected to a maximum intensity projection for analysis. For the APP/BACE-1 complementation assay, DIV 7 neurons were transfected with desired constructs for ˜15-18 h and fixed. z-stack images were captured using a 40× objective (0.339 μm z-step) and subjected to a maximum intensity projection. The average intensity within cell bodies was quantified.

For trafficking studies in axons and dendrites, imaging parameters were set at 1 frame/s and total 200 frames. Kymographs were generated in MetaMorph®, and segmental tracks were traced on the kymographs using a line tool. The resultant velocity (distance/time) and run length data were obtained for each track, frequencies of particle movements were calculated by dividing the number of individual particles moving in a given direction by the total number of analyzed particles in the kymograph, and numbers of particles per minute were calculated by dividing the number of particles moving in a given direction by the total imaging time.

APP endocytosis assay was done as described previously (40). Cells expressing APP-GFP, APP659-GFP, untagged APP or untagged APP-659-GG were starved with serum-free medium for 30 min and incubated with anti-APP (22C11) in complete medium with 10 mM HEPES for 10 min. And then, cells were fixed, permeabilized and immunostained for 22C11. The mean intensity of 22C11 along plasma membrane was calculated by dividing the total intensity along plasma membrane (=intensity of whole cell−intensity of cytoplasm) with area of plasma membrane (=area of whole cell−area of cytoplasm). The ratio of mean intensities between plasma membrane and cytoplasm was quantified.

Stereotactic injection of AAV9s into the mouse brain and histology—All the animal procedures were performed in accordance with University of Wisconsin guidelines. In vivo injection and immunofluorescence staining was done as described previously (41). Briefly, 1.5 μl of 1:2 AAV9 mixture of AAV9-APP sgRNA-GFP (or AAV9-GFP) and AAV9-Cas9 was injected into the dentate gyrus (−2.0, ±1.6, −1.9) of 8-week old male C57BL/6 mice (either sex). 2-weeks after surgery, the mice were sacrificed by trans-cardiac perfusion of saline, followed by 4% PFA. The brains were dissected, post-fixed with 4% PFA overnight, immersed in 30% sucrose until saturation, and sectioned at 40 μm. Sections were immunostained with the following antibodies: mouse anti-HA (1:1000, BioLegend, clone 16B12), chicken anti-GFP (1:1000, Invitrogen, polyclonal) and rabbit anti-APP (1:200, Abcam, clone Y188). Images were acquired using Zeiss LSM800 confocal microscope. Average intensities of APP staining in cell bodies were quantified using Metamorph®.

Intracerebroventricular injections and histology—All animal procedures were approved by the Mayo Institutional Animal Care and Use Committee and are in accordance with the NIH Guide for Care and Use of Laboratory animals. Free hand bilateral intracerebroventricular (ICV) injections were performed as previously described (42) in C57BL/6 mouse pups. On post-natal day 0, newborn pups were briefly cryoanesthetized on ice until no movement was observed. A 30-gauge needle attached to a 10 μl syringe (Hamilton) was used to pierce the skull of the pups just posterior to bregma and 2 mm lateral to the midline. The needle was held at a depth of approximately 2 millimeters, and 2 μl of a mixture of AAV9 viruses (ratio 1:2 of AAV9−-APP sgRNA-GFP or AAV9-GFP+ AAV9-Cas9) were injected into each cerebral ventricle. After 5 minutes of recovery on a heat pad, the pups were returned into their home cages. Mice were sacrificed 15 days after viral injection. Animals were deeply anesthetized with sodium pentobarbital prior to transcardial perfusion with phosphate buffered saline (PBS), and the brain was removed and bisected along the midline. The left hemisphere was drop-fixed in 10% neutral buffered formalin (Fisher Scientific, Waltham, MA) overnight at 4° C. for histology, whereas the right hemisphere of each brain was snap-frozen and homogenized for biochemical analysis. Formalin fixed brains were embedded in paraffin wax, sectioned in a sagittal plane at 5-micron thickness, and mounted on glass slides. Tissue sections were then deparaffinized in xylene and rehydrated. Antigen retrieval was performed by steaming in distilled water for 30 min, followed by permeabilization with 0.5% Triton™-X, and blocking with 5% goat serum for 1 hour. Sagittal sections were then incubated with primary anti-GFP antibody (1:250, Aves, chicken polyclonal) and anti-APP antibody (1:200, Abcam, clone Y188) overnight at 4° C. Sections were incubated with the secondary antibodies Alexa Fluor®-488-goat anti-chicken and Alexa Fluro®-568-goat anti rabbit (1:500, Invitrogen) for 2 h at room temperature. Sections were washed and briefly dipped into 0.3% Sudan Black in 70% ethanol prior to mounting.

Electrophysiology—A coverslip with cultured cells at a density of 60,000 cells/cm² was placed in a continuously perfused bath, viewed under IR-DIC optics and whole-cell voltage clamp recordings were performed (−70 mV, room temp.). The extracellular solution consisted of (in mM): 145 NaCl, 2.5 KCl, 1 MgCl2, 2 CaCl2), 10 HEPES and 10 dextrose, adjusted to 7.3 pH with NaOH and 320 mOsm with sucrose. Whole-cell recordings were made with pipette solutions consisting of (in mM) 140 KCl, 10 EGTA, 10 HEPES, 2 Mg2ATP and 20 phosphocreatine, adjusted to pH 7.3 with KOH and 315 mOsm with sucrose. Excitatory synaptic events were isolated by adding 10 μM bicuculline to block GABA (subscript A) receptors. Miniature synaptic events were isolated by adding 100 nM tetrodotoxin to prevent action potentials. mEPSCs were detected using the template-matching algorithm in Axograph X, with a template that had 0.5 ms rise time and 5 ms decay. Statistics were computed using the Statistics Toolbox of Matlab.

T7 Endonuclease 1 Assay, Off-target, and ICE analyses—Genomic PCR was performed around each sgRNA target, and related off-target sites, following the manufacturer's instruction (using AccuPrime™ HiFi Taq using 500 ng of genomic DNA). Products were then purified using Wizard® SV Gel and PCR Clear-Up System (Promega) and quantified using a Qubit® 2.0 (Thermo Fischer). T7E1 assay was performed according to manufacturer's instructions (New England Biolabs). Briefly, 200 ng of genomic PCR was combined with 2 μL of NEBuffer™ 2 (New England Biolabs) and diluted to 19 μL. Products were then hybridized by denaturing at 95° C. for 5 minutes then ramped down to 85° C. at −2° C./second. This was followed by a second decrease to 25° C. at −0.1° C./second. To hybridized product, 1 μL T7E1 (M0302, New England Biolabs) was added and mixed well followed by incubation at 37° C. for 15 minutes. Reaction was stopped by adding 1.5 μL of 0.25M EDTA. Products were analyzed on a 3% agarose gel and quantified using a Gel Doc XR system (BioRad). Off-target sites were identified and scored using Benchling. The top 5 off-target sites—chosen on the basis of raw score and irrespective of being in a coding region—were identified and analyzed using T7E1 assay as previously described. For TIDE (43), PCR was performed on genomic DNA using Accuprime™ Taq HiFi (Thermo Fischer) according to manufacture specifications. Briefly, reactions were cycled at 2 min at 94° C. followed by 35 cycles of 98° C. for 30 seconds, 58° C. for 30 seconds, and 68° C. for 2 minutes 30 seconds and a final extension phase of 68° C. for 10 minutes. Products were then subjected to Sanger Sequencing and analyzed using the TIDE platform. The primers used for TIDE analyses are listed in Table 3. For analyses of indel after CRISPR editing with APP670-sgRNA and APP676-sgRNA, the edited regions of genomic DNA were PCR amplified and subjected to Sanger Sequencing. The results were analyzed using the ICE platform.

Deep Sequencing Sample Preparation and data analysis—Genomic PCR was performed using AccuPrime™ HiFi Taq (Life Technologies) following manufacturer's instructions. About 200-500 ng of genomic DNA was used for each PCR reaction. Products were then purified using AMPure® XP magnetic bead purification kit (Beckman Coulter) and quantified using a Nanodrop2000. Individual samples were pooled and run on an Illumina® HiSeq2500 High Throughput at a run length of 2×125 bp. A custom python script was developed to perform sequence analysis. For each sample, sequences with frequency of less than 100 reads were filtered from the data. Sequences in which the reads matched with primer and reverse complement subsequences classified as target sequences. These sequences were then aligned with corresponding wildtype sequence using global pairwise sequence alignment. Sequences that were misaligned through gaps or insertions around the expected cut site were classified as NHEJ events. The frequency, length, and position of matches, insertions, deletions, and mismatches were all tracked in the resulting aligned sequences.

Statistical analysis—Statistical analysis was performed and plotted using Prism software. Student's t-test (unpaired, two-tailed) was used to compare two groups. One-way ANOVA test was used to compare multiple groups, following with Tukey multiple comparison test of every pair. A P-value <0.05 was considered significant.

TABLE 3
PCR PRIMERS USED FOR ON- AND OFF- TARGET GENOMIC LOCI AMPLIFICATION

| | Forward primer | SEQ ID | Reverse primer | SEQ ID |
|---|---|---|---|---|
| | sequence (5′-3′) | NO: | sequence (5′-3′) | NO: |
| Mouse | AGGAACGGAGTGACCTGTTTCC | 21 | TTCCTCCATGGTAACCACGCAT | 22 |
| APP | | | | |
| (659) | | | | |
| Human | TGGGGAAGCCACATGTTGTACA | 23 | ATGTTTTGGTGGGCCATTTGGT | 24 |
| APP | | | | |
| (659) | | | | |
| Human | AAATTATGGGTGTTCTGCAATCTTGG | 25 | ACTTGTGTTACAGCACAGCTGTC | 26 |
| APP | | | | |
| (670; | | | | |
| 676) | | | | |
| Mouse | GCCCTCCAGAAGTATTGGCTT | 27 | GTCAGGGCCTTGCTCTACAAA | 28 |
| OT1 | | | | |
| Mouse | CGCAAAAACTGGCTGCGTAT | 29 | TGTAGGCGCACATGCAGAAG | 30 |
| OT2 | | | | |
| Mouse | CAGGTAGAGCGTGGAAACTCA | 31 | TGTGCGCATTAGGACCAGAT | 32 |
| OT3 | | | | |
| Mouse | CACCTGACAATGCTGTCCCA | 33 | AGACAAGGTCTGTCTCCTTGC | 34 |
| OT4 | | | | |
| Mouse | CCAACTCTTTGCTTAGGGGC | 35 | ATCGTCCCTGGTGCATTCTC | 36 |
| OT5 | | | | |
| Human | GGAAAACCAGGTAGAGGGGG | 37 | TCTCTGGCTCGAGGGTACAT | 38 |
| OT1 | | | | |
| Human | CTGCATGCCATGGGTAGGTA | 39 | CAGGCTGTTTCGGGTCCTT | 40 |
| OT2 | | | | |
| Human | AGACTCTTCTCCGATTCCAGC | 41 | TCCAGCACGATCTGGTAGGC | 42 |
| OT3 | | | | |
| Human | AGTGCTTTTCTTTGCCTTTGCT | 43 | TGCTCGGGAGGTGTTTCTAC | 44 |
| OT4 | | | | |
| Human | AACAAGGCAGCTCCTCAACT | 45 | GACGTCAGAATTGAGGGTGGA | 46 |
| OT5 | | | | |
| Mouse | CCAGCGGGATGAACTGGTAAGA | 47 | CCCAGGTCACCTTAAGGAGCAA | 48 |
| APLP1 | | | | |
| Mouse | GAGAGAGTTGGAGGCCTTGAGG | 49 | AACCACAGTGACAAGTGGCTCT | 50 |
| APLP2 | | | | |
| Human | GTGAATGCGTCTGTTCCAAGGG | 51 | GCTGCTGGGACTATCTGGGAAT | 52 |
| APLP1 | | | | |
| Human | TTTTAGGGGCTCGACCTTCCAG | 53 | TGCACTAATTTCCCAGGGCTCA | 54 |
| APLP2 | | | | |

TABLE 4
TRANSPORT PARAMETERS OF WT AND APP659

| | | | | | | |
|---|---|---|---|---|---|---|
| | | | | | Anterograde | |
| | | | | Retrograde | velocity | Retrograde |
| | | | Anterograde | Run- | (μm/sec), | velocity |
| % | % | % | Run-length | length | mean ± | (μm/sec), |
| Anterograde | Retrograde | Stationary | (μm) | (μm) | SEM | mean ± SEM |

Kinetics in axons

| | | | | | | | |
|---|---|---|---|---|---|---|---|
| APP659 | 53.88 ± 4.57 | 37.13 ± 4.36 | 8.97 ± 1.51 | 8.08 ± 0.31 | 6.8 ± 0.26 | 1.66 ± 0.03 | 1.52 ± 0.03 |
| APPWT | 57.84 ± 2.22 | 34.37 ± 4.46 | 10.42 ± 4.24 | 10.44 ± 0.42 | 6.35 ± 0.26 | 1.97 ± 0.03 | 1.52 ± 0.03 |

Kinetics in dendrites

| | | | | | | | |
|---|---|---|---|---|---|---|---|
| APP659 | 37.81 ± 6.45 | 20.55 ± 6.21 | 41.64 ± 8.37 | 7.0 ± 0.48 | 6.94 ± 0.81 | 0.76 ± 0.05 | 0.83 ± 0.12 |
| APPWT | 45.83 ± 4.58 | 24.81 ± 2.97 | 29.35 ± 5.63 | 8.12 ± 0.46 | 7.98 ± 0.94 | 0.94 ± 0.03 | 0.9 ± 0.07 |

~115 APP659:GFP and ~130 APP:GFP vesicles analyzed in dendrites;
~310 APP659:GFP and ~325 APP:GFP vesicles in axons (from 10-12 neurons from 2 separate cultures.)

TABLE 5
APP SGRNAS TARGETING SEQUENCES

| | sgRNA targeting sequence | SEQ ID NO: |
|---|---|---|
| Human APP 659 | ATCCATTCATCATGGTGTGG | 1 |
| Human APP 670 | TGGACAGGTGGCGCTCCTCT | 2 |
| Human APP 676 | GTAGCCGTTCTGCTGCATCT | 4 |
| Mouse APP 659 | ATCCATCCATCATGGCGTGG | 6 |
| Mouse APP 670 | TGGAGAGATGGCGCTCCTCT | 7 |
| Mouse APP 676 | ATATCCGTTCTGCTGCATCT | 9 |

### REFERENCES

1) Fellmann, C., Gowen, B. G., Lin, P. C., Doudna, J. A. & Corn, J. E. Cornerstones of CRISPR-Cas in drug discovery and therapy. Nat Rev Drug Discov 16, 89-100, doi:10.1038/nrd.2016.238 (2017).
2) Yang, S. et al. CRISPR/Cas9-mediated gene editing ameliorates neurotoxicity in mouse model of Huntington's disease. J Clin Invest 127, 2719-2724, doi:10.1172/JCI92087 (2017).
3) Park, C. Y. et al. Reversion of FMR1 Methylation and Silencing by Editing the Triplet Repeats in Fragile X iPSC-Derived Neurons. Cell Rep 13, 234-241, doi: 10. 1016/j.celrep.2015.08.084 (2015).
4) Gyorgy, B. et al. CRISPR/Cas9 Mediated Disruption of the Swedish APP Allele as a Therapeutic Approach for Early-Onset Alzheimer's Disease. Mol Ther Nucleic Acids 11, 429-440, doi:10.1016/j.omtn.2018.03.007 (2018).
5) McMahon, M. A. & Cleveland, D. W. Gene therapy: Gene-editing therapy for neurological disease. Nat Rev Neurol 13, 7-9, doi:10.1038/nrneurol.2016.190 (2017).
6) Mockett, B. G., Richter, M., Abraham, W. C. & Muller, U. C. Therapeutic Potential of Secreted Amyloid Precursor Protein APPsalpha. Front Mol Neurosci 10, 30, doi:10.3389/fnmol.2017.00030 (2017).
7) O'Brien, R. J. & Wong, P. C. Amyloid precursor protein processing and Alzheimer's disease. Annu Rev Neurosci 34, 185-204, doi:10.1146/annurev-neuro-061010-113613 (2011).
8) Fol, R. et al. Viral gene transfer of APPsalpha rescues synaptic failure in an Alzheimer's disease mouse model. Acta Neuropathol 131, 247-266, doi:10.1007/s00401-015-1498-9 (2016).
9) Richter, M. C. et al. Distinct in vivo roles of secreted APP ectodomain variants APPsalpha and APPsbeta in regulation of spine density, synaptic plasticity, and cognition. EMBO J 37, doi:10.15252/embj.201798335 (2018).
10) Das, U. et al. Visualizing APP and BACE-1 approximation in neurons yields insight into the amyloidogenic pathway. Nat Neurosci 19, 55-64, doi:10.1038/nn.4188 (2016).
11) Citron, M., Teplow, D. B. & Selkoe, D. J. Generation of amyloid beta protein from its precursor is sequence specific. Neuron 14, 661-670, doi:0896-6273(95)90323-2 [pii](1995).
12) Koo, E. H. & Squazzo, S. L. Evidence that production and release of amyloid beta-protein involves the endocytic pathway. J Biol Chem 269, 17386-17389 (1994).
13) Ring, S. et al. The secreted beta-amyloid precursor protein ectodomain APPs alpha is sufficient to rescue the anatomical, behavioral, and electrophysiological abnormalities of APP-deficient mice. J Neurosci 27, 7817-7826, doi:10.1523/JNEUROSCI.1026-07.2007 (2007).
14) Sander, J. D. & Joung, J. K. CRISPR-Cas systems for editing, regulating and targeting genomes. Nat Biotechnol 32, 347-355, doi:10.1038/nbt.2842 (2014).
15) Muller, U. C. & Zheng, H. Physiological functions of APP family proteins. Cold Spring Harb Perspect Med 2, a006288, doi:10.1101/cshperspect.a006288 (2012).
16) Muller, U. C., Deller, T. & Korte, M. Not just amyloid: physiological functions of the amyloid precursor protein family. Nat Rev Neurosci 18, 281-298, doi:10.1038/nrn.2017.29 (2017).
17) Carlson-Stevermer, J. et al. Assembly of CRISPR ribonucleoproteins with biotinylated oligonucleotides via an RNA aptamer for precise gene editing. Nat Commun 8, 1711, doi:10.1038/s41467-017-01875-9 (2017).
18) Deyts, C., Thinakaran, G. & Parent, A. T. APP Receptor? To Be or Not To Be. Trends Pharmacol Sci 37, 390-411, doi:10.1016/j.tips.2016.01.005 (2016).
19) Beckett, C., Nalivaeva, N. N., Belyaev, N. D. & Turner, A. J. Nuclear signalling by membrane protein intracellular domains: the AICD enigma. Cell Signal 24, 402-409, doi:10.1016/j.cellsig.2011.10.007 (2012).
20) Pardossi-Piquard, R. & Checler, F. The physiology of the beta-amyloid precursor protein intracellular domain AICD. J Neurochem 120 Suppl 1, 109-124, doi:10.1111/j.1471-4159.2011.07475. x (2012).
21) Passini, M. A. & Wolfe, J. H. Widespread gene delivery and structure-specific patterns of expression in the brain after intraventricular injections of neonatal mice with an adeno-associated virus vector. J Virol 75, 12382-12392, doi:10.1128/JVI.75.24.12382-12392.2001 (2001).
22) Kim, J. Y., Grunke, S. D. & Jankowsky, J. L. Widespread Neuronal Transduction of the Rodent CNS via Neonatal Viral Injection. Methods Mol Biol 1382, 239-250, doi:10.1007/978-1-4939-3271-9_17 (2016).
23) Lee, M. S. et al. APP processing is regulated by cytoplasmic phosphorylation. J Cell Biol 163, 83-95, doi:10.1083/jcb.200301115 (2003).
24) Perez, R. G. et al. Mutagenesis identifies new signals for beta-amyloid precursor protein endocytosis, turnover, and the generation of secreted fragments, including Abeta42. J Biol Chem 274, 18851-18856 (1999).
25) Lai, A., Sisodia, S. S. & Trowbridge, I. S. Characterization of sorting signals in the beta-amyloid precursor protein cytoplasmic domain. J Biol Chem 270, 3565-3573 (1995).
26) Thinakaran, G. & Koo, E. H. Amyloid precursor protein trafficking, processing, and function. J Biol Chem 283, 29615-29619, doi:10.1074/jbc.R800019200 (2008).
27) Vassar, R. et al. Function, therapeutic potential and cell biology of BACE proteases: current status and future prospects. J Neurochem 130, 4-28, doi:10.1111/jnc.12715 (2014).
28) Morel, E. et al. Phosphatidylinositol-3-phosphate regulates sorting and processing of amyloid precursor protein through the endosomal system. Nat Commun 4, 2250, doi:10.1038/ncomms3250 (2013).
29) Das, U. et al. Activity-induced convergence of APP and BACE-1 in acidic microdomains via an endocytosis-dependent pathway. Neuron 79, 447-460, doi:10.1016/j.neuron.2013.05.035 (2013).
30) Sisodia, S. S. Beta-amyloid precursor protein cleavage by a membrane-bound protease. Proc Natl Acad Sci USA 89, 6075-6079 (1992).
31) Chow, V. W., Mattson, M. P., Wong, P. C. & Gleichmann, M. An overview of APP processing enzymes and products. Neuromolecular Med 12, 1-12, doi:10.1007/s12017-009-8104-z (2010).
32) Sun, J. & Roy, S. The physical approximation of APP and BACE-1: A key event in alzheimer's disease pathogenesis. Dev Neurobiol 78, 340-347, doi:10.1002/dneu.22556 (2018).
33) Swiech, L. et al. In vivo interrogation of gene function in the mammalian brain using CRISPR-Cas9. Nat Biotechnol 33, 102-106, doi:10.1038/nbt.3055 (2015).
34) Sanjana, N. E., Shalem, O. & Zhang, F. Improved vectors and genome-wide libraries for CRISPR screening. Nat Methods 11, 783-784, doi:10.1038/nmeth.3047 (2014).
35) Joung, J. et al. Genome-scale CRISPR-Cas9 knockout and transcriptional activation screening. Nat Protoc 12, 828-863, doi:10.1038/nprot.2017.016 (2017).
36) Topol, A., Tran, N. N. & Brennand, K. J. A guide to generating and using hiPSC derived NPCs for the study of neurological diseases. J Vis Exp, e52495, doi:10.3791/52495 (2015).
37) Aime, P. et al. Trib3 Is Elevated in Parkinson's Disease and Mediates Death in Parkinson's Disease Models. J Neurosci 35, 10731-10749, doi:10.1523/JNEUROSCI.0614-15.2015 (2015).
38) Paquet, D. et al. Efficient introduction of specific homozygous and heterozygous mutations using CRISPR/Cas9. Nature 533, 125-+, doi:10.1038/nature17664 (2016).
39) Tang, Y. et al. Early and selective impairments in axonal transport kinetics of synaptic cargoes induced by soluble amyloid beta-protein oligomers. Traffic 13, 681-693, doi:10.1111/j.1600-0854.2012.01340.x (2012).
40) Ubelmann, F. et al. Bin1 and CD2AP polarise the endocytic generation of beta-amyloid. EMBO Rep 18, 102-122, doi:10.15252/embr.201642738 (2017).
41) Guo, W. et al. Fragile X Proteins FMRP and FXR2P Control Synaptic GluA1 Expression and Neuronal Maturation via Distinct Mechanisms. Cell Rep 11, 1651-1666, doi:10.1016/j.celrep.2015.05.013 (2015).
42) Chakrabarty, P. et al. Capsid serotype and timing of injection determines AAV transduction in the neonatal mice brain. PLoS One 8, e67680, doi:10.1371/journal.pone.0067680 (2013).
43) Brinkman, E. K., Chen, T., Amendola, M. & van Steensel, B. Easy quantitative assessment of genome editing by sequence trace decomposition. Nucleic Acids Res 42, e168, doi:10.1093/nar/gku936 (2014).

1. A method of treating or pre-treating a patient having or at risk of forming amyloid plaques composed of amyloid beta (Aβ) peptides, wherein the method comprises the steps of
(a) obtaining a gene-editing construct specific for the amyloid precursor protein (APP), wherein the construct facilitates truncation of the APP C-terminus when combined with a Cas9 nuclease, and
(b) delivering the construct and a construct encoding the Cas9 nuclease to a patient in need of AD therapy, wherein the APP molecule is truncated and production of Aβ peptides is decreased in the patient's brain.

2. The method of claim 1, wherein the truncation of the APP C-terminus occurs at an APP residue selected from the group consisting of 659, 670, 676, and 686.

3. The method of claim 1, wherein the gene-editing construct comprises a gRNA sequence selected from the group consisting of SEQ ID NOs:1-10.

4. The method of claim 1, wherein the construct and the nuclease are delivered in a composition comprising an adeno-associated viral vector and a nanocarrier delivery vehicle.

5. The method of claim 4, wherein the composition is delivered intravenously or intrathecally.
edit endogenous amyloid precursor protein (APP).

6. A method of editing endogenous amyloid precursor protein (APP), the method comprising the steps of
(a) obtaining a gene-editing construct specific for APP, wherein the construct facilitates truncation of the APP C-terminus when combined with a Cas9 nuclease, and
(b) delivering the construct and nuclease to a patient in need of AD therapy, wherein the APP molecule is truncated and production of Aβ peptides is decreased in the patient's brain.

7. The method of claim 6, wherein the truncation of the APP C-terminus occurs at an APP residue selected from the group consisting of 659, 670, 676, and 686.

8. The method of claim 6, wherein the gene-editing construct comprises a gRNA sequence selected from the group consisting of SEQ ID NO:1-10.

9. The method of claim 6, wherein the construct and the nuclease are delivered in a composition comprising an adeno-associated viral vector and a nanocarrier delivery vehicle.

10. The method of claim 6, wherein the composition is delivered intravenously or intrathecally.
